(19) United States
(12) Patent Application Publication
(10) Pub. No.: US 2026/0097117 A1
(43) Pub. Date: Apr. 9, 2026

(54) CONJUGATES

(71) Applicants: Orum Therapeutics, Inc., Yuseong-gu (KR); BRISTOL-MYERS SQUIBB COMPANY, Lawrenceville, NJ (US)

(72) Inventors: Nathan FISHKIN, Cambridge, MA (US); Peter U. PARK, Cambridge, MA (US)

(21) Appl. No.: 19/348,414

(22) Filed: Oct. 2, 2025

Related U.S. Application Data
(63) Continuation of application No. 17/995,005, now abandoned.
(60) Provisional application No. 63/003,190, filed on Mar. 31, 2020.

(51) Int. Cl. A61K 39/39; A61K 39/00; A61K 47/68; C07D 409/14

(52) U.S. Cl. CPC A61K 39/39; A61K 39/0011; A61K 47/6803; A61K 47/6849; A61K 47/6855; A61K 47/6889; C07D 409/14

(57) ABSTRACT

The present disclosure provides conjugates comprising a binding moiety and an immunomodulatory imide compound, e.g., substituted isoindoline compound, wherein the immunomodulatory imide compound is conjugated to a binding moiety via an optional linker. Also provided are compositions comprising the conjugates. The conjugates and compositions are useful for treating a disease or condition, e.g., cancer, in a subject in need thereof.

### FIELD

The present disclosure provides conjugates comprising a binding moiety and an immunomodulatory imide compound, e.g., substituted isoindoline compound, wherein the immunomodulatory imide compound is conjugated to a binding moiety via an optional linker. Also provided are compositions comprising the conjugates. The conjugates and compositions are useful for treating cancer in a subject in need thereof.

### BACKGROUND

Protein degradation has been validated as a therapeutic strategy by the effectiveness of immunomodulatory imide drugs (known as IMiDs). These compounds have the ability to bind to cereblon (CRBN) and promote recruitment and ubiquitination of substrate proteins mediated by CRL4^{CRBN} E3 ubiquitin ligase. It is thought that IMiDs act as “molecular glues,” filling the binding interface as a hydrophobic patch that reprograms protein interactions between the ligase and neosubstrates.

Despite the excitement for IMiDs as novel treatments for cancer, thus far they have been limited to use in hematologic malignancies such as multiple myeloma and myelodysplastic syndrome (MDS). Thus there is a continuing need for new IMiDs conjugates that can target alternative oncoproteins and treat a wide array of cancers.

### SUMMARY

The present disclosure provides a conjugate comprising a binding moiety that specifically binds to a protein, and an immunomodulatory imide compound, wherein the binding moiety and the immunomodulatory imide compound are linked via an optional linker (L).

The present disclosure further provides a method of preparing said conjugate, or a pharmaceutically acceptable salt thereof, the process comprising reacting a binding moiety with a structure L′-V, wherein:
V is a 5-substituted isoindoline compound; and
L′ is a cleavable or non-cleavable linker precursor that conjugates to the binding moiety.

### DETAILED DESCRIPTION

The present disclosure is directed to a conjugate comprising a binding moiety and an immunomodulatory imide compound, wherein the binding moiety and the immunomodulatory imide compound are linked via an optional linker. In some aspects, the conjugate comprises formula (I):

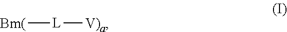

or a pharmaceutically acceptable salt thereof, wherein:
   a is an integer from 1 to 10;
   V is a substituted isoindoline compound;
   L is a cleavable linker or non-cleavable linker; and
   Bm is a binding moiety that is capable of specifically binding to a protein.

The present disclosure also provides the composition comprising the conjugate, the method of using or making the conjugate, or methods of treating or preventing a disease or condition using the conjugate.

### I. Definition

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “antibody,” as used herein, also refers to a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

The term “single domain antibody,” also known as a nanobody, is an antibody fragment consisting of a single monomeric variable antibody domain with a molecular weight of from about 12 kDa to about 15 kDa. Single body antibodies can be based on heavy chain variable domains or light chains. Examples of single domain antibodies include, but are not limited to, V_{H}H fragments and V_{NAR} fragments.

“Antibody fragments” comprise a portion of an intact antibody, generally the antigen binding or variable region thereof. Examples of antibody fragments include Fab, Fab′, F(ab′).sub.2, and Fv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

An “intact antibody” is one which comprises an antigen-binding variable region as well as a light chain constant domain (CL) and heavy chain constant domains, CH1, CH2 and CH3. The constant domains may be native sequence constant domains (e.g., human native sequence constant domains) or amino acid sequence variant thereof.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method, or may be made by recombinant DNA methods. The “monoclonal antibodies” may also be isolated from phage antibody libraries.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies of interest herein include “primatized” antibodies comprising variable domain antigen-binding sequences derived from a non-human primate (e.g., Old World Monkey, Ape etc.) and human constant region sequences.

Various methods have been employed to produce monoclonal antibodies (MAbs). Hybridoma technology, which refers to a cloned cell line that produces a single type of antibody, uses the cells of various species, including mice (murine), hamsters, rats, and humans. Another method to prepare MAbs uses genetic engineering including recombinant DNA techniques. Monoclonal antibodies made from these techniques include, among others, chimeric antibodies and humanized antibodies. A chimeric antibody combines DNA encoding regions from more than one type of species. For example, a chimeric antibody may derive the variable region from a mouse and the constant region from a human. A humanized antibody comes predominantly from a human, even though it contains nonhuman portions. Like a chimeric antibody, a humanized antibody may contain a completely human constant region. But unlike a chimeric antibody, the variable region may be partially derived from a human. The nonhuman, synthetic portions of a humanized antibody often come from CDRs in murine antibodies. In any event, these regions are crucial to allow the antibody to recognize and bind to a specific antigen. While useful for diagnostics and short-term therapies, murine antibodies cannot be administered to people long-term without increasing the risk of a deleterious immunogenic response. This response, called Human Anti-Mouse Antibody (HAMA), occurs when a human immune system recognizes the murine antibody as foreign and attacks it. A HAMA response can cause toxic shock or even death.

Chimeric and humanized antibodies reduce the likelihood of a HAMA response by minimizing the nonhuman portions of administered antibodies. Furthermore, chimeric and humanized antibodies can have the additional benefit of activating secondary human immune responses, such as antibody dependent cellular cytotoxicity.

The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g., B cell receptor; BCR), etc.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called .alpha., .delta., .epsilon., .gamma., and .mu., respectively. The subunit structures and three-dimensional configurations of different classes of immunoglobulins are well known.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The terms “administration,” “administering,” and grammatical variants thereof refer to introducing a composition, such as an EV (e.g., exosome) of the present disclosure, into a subject via a pharmaceutically acceptable route. The introduction of a composition, such as an EV (e.g., exosome) of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function. In some aspects of the present disclosure, the biologically active molecule is an antibody or a molecule comprising an antigen binding fragment thereof.

The terms “antibody-drug conjugate” and “ADC” are used interchangeably and refer to an antibody linked, e.g., covalently, to a therapeutic agent (sometimes referred to herein as agent, drug, or active pharmaceutical ingredient) or agents. In some aspects of the present disclosure, the biologically active molecule is an antibody-drug conjugate.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least about 70% identical, at least about 80% identical, at least about 90% identical, or at least about 95% identical to one another. In some aspects, two or more sequences are said to be “conserved” if they are at least about 30% identical, at least about 40% identical, at least about 50% identical, at least about 60% identical, at least about 70% identical, at least about 80% identical, at least about 90% identical, or at least about 95% identical to one another. Conservation of sequence can apply to the entire length of an polynucleotide or polypeptide or can apply to a portion, region or feature thereof.

As used herein, the terms “linking” and “conjugating” are used interchangeably an each refer to the covalent or non-covalent attachment of two or more moieties comprising an immunomodulatory imide compound, e.g., substituted isoindoline compound and a binding moiety. In some aspects the linking or conjugating can comprise a linker.

The term “amino acid sequence variant” refers to polypeptides having amino acid sequences that differ to some extent from a native sequence polypeptide. Ordinarily, amino acid sequence variants will possess at least about 70% sequence identity with at least one receptor binding domain of a native antibody or with at least one ligand binding domain of a native receptor, and typically, they will be at least about 80%, more typically, at least about 90% homologous by sequence with such receptor or ligand binding domains. The amino acid sequence variants possess substitutions, deletions, and/or insertions at certain positions within the amino acid sequence of the native amino acid sequence. Amino acids are designated by the conventional names, one-letter and three-letter codes.

“Sequence identity” is defined as the percentage of residues in the amino acid sequence variant that are identical after aligning the sequences and introducing gaps, if necessary, to achieve the maximum percent sequence identity. Methods and computer programs for the alignment are well known in the art. One such computer program is “Align 2,” authored by Genentech, Inc., which was filed with user documentation in the United States Copyright Office, Washington, D.C. 20559, on Dec. 10, 1991.

The terms “Fc receptor” or “FcR” are used to describe a receptor that binds to the Fc region of an antibody. An exemplary FcR is a native sequence human FcR. Moreover, a FcR may be one which binds an IgG antibody (a gamma receptor) and includes receptors of the Fc.gamma.RI, Fc.gamma.RII, and Fc.gamma. RIII subclasses, including allelic variants and alternatively spliced forms of these receptors. Fc.gamma.RII receptors include Fc.gamma.RIIA (an “activating receptor”) and Fc.gamma.RIIB (an “inhibiting receptor”), which have similar amino acid sequences that differ primarily in the cytoplasmic domains thereof. Activating receptor Fc.gamma.RIIA contains an immunoreceptor tyrosine-based activation motif (ITAM) in its cytoplasmic domain. Inhibiting receptor Fc.gamma.RIIB contains an immunoreceptor tyrosine-based inhibition motif (ITIM) in its cytoplasmic domain. Other FcRs, including those to be identified in the future, are encompassed by the term “FcR” herein. The term also includes the neonatal receptor, FcRn, which is responsible for the transfer of maternal IgGs to the fetus.

“Complement dependent cytotoxicity” or “CDC” refers to the ability of a molecule to lyse a target in the presence of complement. The complement activation pathway is initiated by the binding of the first component of the complement system (C1q) to a molecule (e.g., an antibody) complexed with a cognate antigen. To assess complement activation, a CDC assay may be performed.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a .beta.-sheet configuration, connected by three hypervariable regions, which form loops connecting, and in some cases forming part of, the .beta.-sheet structure. The hypervariable regions in each chain are held together in close proximity by the FRs and, with the hypervariable regions from the other chain, contribute to the formation of the antigen-binding site of antibodies. The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (H3) in the heavy chain variable domain; Kabat et al supra) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain). “Framework Region” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (.kappa.) and lambda (.lamda.), based on the amino acid sequences of their constant domains.

“Single-chain Fv” or “scFv” antibody fragments comprise the VH and VL domains of antibody, wherein these domains are present in a single polypeptide chain. The Fv polypeptide may further comprise a polypeptide linker between the VH and VL domains which enables the scFv to form the desired structure for antigen binding.

The term “diabodies” refers to small antibody fragments with two antigen-binding sites, which fragments comprise a variable heavy domain (VH) connected to a variable light domain (VL) in the same polypeptide chain (VH-VL). By using a linker that is too short to allow pairing between the two domains on the same chain, the domains are forced to pair with the complementary domains of another chain and create two antigen-binding sites.

“Humanized” forms of non-human (e.g., rodent) antibodies are chimeric antibodies that contain minimal sequence derived from non-human immunoglobulin. Humanization is a method to transfer the murine antigen binding information to a non-immunogenic human antibody acceptor, and has resulted in many therapeutically useful drugs. The method of humanization generally begins by transferring all six murine complementarity determining regions (CDRs) onto a human antibody framework. These CDR-grafted antibodies generally do not retain their original affinity for antigen binding, and in fact, affinity is often severely impaired. Besides the CDRs, select non-human antibody framework residues must also be incorporated to maintain proper CDR conformation. The transfer of key mouse framework residues to the human acceptor in order to support the structural conformation of the grafted CDRs has been shown to restore antigen binding and affinity. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In certain aspects, the antibody will be purified (1) to greater than 95% by weight of antibody as determined by the Lowry method, or more than 99% by weight, (2) to a degree sufficient to obtain at least 15 residues of N-terminal or internal amino acid sequence by use of a gas phase protein sequencer, or (3) to homogeneity by SDS-PAGE under reducing or nonreducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell. As used herein, the term “T cell” and “T lymphocytes” are interchangeable and refer to any lymphocytes produced or processed by the thymus gland. In some aspects, a T cell is a CD4+ T cell. In some aspects, a T cell is a CD8+ T cell. In some aspects, a T cell is a NKT cell.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein.

The term “therapeutically effective amount” or “therapeutically effective dosage” refers to an amount of a conjugate disclosed herein that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the composition can, for example, (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer.

In some aspects, a “therapeutically effective amount” is the amount of the conjugate clinically proven to affect a significant decrease in cancer or slowing of progression (regression) of cancer, such as an advanced solid tumor. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the term “standard of care” refers to a treatment that is accepted by medical experts as a proper treatment for a certain type of disease and that is widely used by healthcare professionals. The term can be used interchangeable with any of the following terms: “best practice,” “standard medical care,” and “standard therapy.”

By way of example, an “anti-cancer agent” promotes cancer regression in a subject or prevents further tumor growth. In certain aspects, a therapeutically effective amount of the drug promotes cancer regression to the point of eliminating the cancer.

The terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

As used herein, the term “immune checkpoint inhibitor” refers to molecules that totally or partially reduce, inhibit, interfere with or modulate one or more checkpoint proteins. Checkpoint proteins regulate T-cell activation or function. Numerous checkpoint proteins are known, such as CTLA-4 and its ligands CD80 and CD86; and PD-1 with its ligands PD-L1 and PD-L2. Pardoll, D. M., Nat Rev Cancer 12(4):252-64 (2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies.

The terms “treat” or “treatment” refer to both therapeutic treatment and prophylactic or preventative measures, wherein the object is to prevent or slow down (lessen) an undesired physiological change or disorder, such as the development or spread of cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The term “solvate” means a compound in the present disclosure or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise —NO, —NO₂, —ONO, or —ONO₂ moieties. Prodrugs can typically be prepared using well-known methods, such as those described in Burger's Medicinal Chemistry and Drug Discovery, 172-178, 949-982 (Manfred E. Wolff ed., 5th ed. 1995), and Design of Prodrugs (H. Bundgaard ed., Elselvier, New York 1985).

The term “stereoisomer” encompasses all enantiomerically/stereomerically pure and enantiomerically/stereomerically enriched compounds in this disclosure.

The term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

The term “stereomerically enriched” means a composition that comprises greater than about 55% by weight of one stereoisomer of a compound, greater than about 60% by weight of one stereoisomer of a compound, preferably greater than about 70% by weight, more preferably greater than about 80% by weight of one stereoisomer of a compound.

The term “enantiomerically pure” means a stereomerically pure composition of a compound having one chiral center. Similarly, the term “enantiomerically enriched” means a stereomerically enriched composition of a compound having one chiral center.

The term “halogen”, “halide” or “halo” refers to fluorine, chlorine, bromine, and/or iodine.

The term “alkyl” refers to a saturated straight chain or branched hydrocarbon having number of carbon atoms as specified herein. Representative saturated straight chain alkyls include -methyl, -ethyl, -n-propyl, -n-butyl, -n-pentyl, and -n-hexyl; while saturated branched alkyls include -isopropyl, -.sec-butyl, -isobutyl, -tert-butyL, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, and the like. The term “alkyl” also encompasses cycloalkyl.

The term “cycloalkyl” means a specie of alkyl containing from 3 to 15 carbon atoms, without alternating or resonating double bonds between carbon atoms. It may contain from 1 to 4 rings. Examples of unsubstituted cycloalkyls include, but are not limited to, cyclopropyl, cyclobutyl, cyclopentyl, cyclohexyl, and adamantyl. A cycloalkyl may be substituted with one or more of the substituents as defined below.

The term “alkoxy” refers to —O-(alkyl), wherein alkyl is defined herein. Examples of alkoxy include, but are not limited to, —OCH₃, —OCH₂CH₃, —O(CH₂)₂CH₃, —O(CH₂)₃CH₃, —O(CH₂)₄CH₃, and —O(CH₂)₅CH₃.

The term “aryl” means a carbocyclic aromatic ring containing from 5 to 14 ring atoms. The ring atoms of a carbocyclic aryl group are all carbon atoms. Aryl ring structures include compounds having one or more ring structures such as mono-, bi-, or tricyclic compounds as well as benzo-fused carbocyclic moieties such as 5,6,7,8-tetrahydronaphthyl and the like. Representative aryl groups include phenyl, anthracenyl, fluorenyl, indenyl, azulenyl, phenanthrenyl and naphthyl.

The term “heteroaryl” means an aromatic ring containing from 5 to 14 ring atoms, of which at least one (e.g., one, two, or three) is a heteroatom (e.g., nitrogen, oxygen, or sulfur). Heteroaryl ring structures include compounds having one or more ring structures such as mono-, bi-, or tricyclic compounds, as well as fused heterocyclic moieties. Examples of heteroaryls include, but are not limited to, triazolyl, tetrazolyl, oxadiazolyl, pyridyl, furyl, benzofuranyl, thiophenyl, thiazolyl, benzothiophenyl, benzoisoxazolyl, benzoisothiazolyl, quinolinyl, isoquinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, benzoquinazolinyl, quinoxalinyl, acridinyl, pyrimidyl, oxazolyl, benzo[1,3]dioxole and 2,3-dihydro-benzo[1,4]dioxine.

The term “heterocycle” means a monocyclic or polycyclic ring comprising carbon and hydrogen atoms, optionally having 1 or 2 multiple bonds, and the ring atoms contain at least one heteroatom, specifically 1 to 3 heteroatoms, independently selected from nitrogen, oxygen, and sulfur. Heterocycle ring structures include, but are not limited to, mono-, bi-, and tri-cyclic compounds. Specific heterocycles are monocyclic or bicyclic. Representative heterocycles include morpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl and tetrahydrothiopyranyl. A heterocyclic ring may be unsubstituted or substituted.

The term “heterocycloalkyl” refers to a cycloalkyl group in which at least one of the carbon atoms in the ring is replaced by a heteroatom (e.g., O, S or N).

### I. Conjugates

The present disclosure provides conjugates comprising one or more immunomodulatory imide compound and a binding moiety. These conjugates can degrade proteins by binding to cereblon (CRBN), promoting recruitment and ubiquitination of substrate proteins mediated by CRL4^{CRBN} E3 ubiquitin ligase. Immunomodulatory imide compounds are a class of immunomodulatory compounds (drugs that adjust immune responses) containing an imide group.

In some aspects, the present disclosure provides a compound of formula (I),

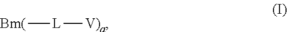

or a pharmaceutically acceptable salt thereof, wherein:
   a is an integer from 1 to 10;
   V is a substituted isoindoline compound;
   L is a cleavable linker or non-cleavable linker; and
   Bm is a binding moiety that is capable of specifically binding to a protein. In some aspects, V is a 5′ substituted isoindoline compound.

In some aspects, the conjugate described herein has in vitro anti-proliferative activity against a tumor cell line. In some aspects, the conjugate comprising an immunomodulatory imide compound, e.g., 5′-substituted isoindoline compound, and a binding moiety has in vitro anti-proliferative activity at least about 50%, at least about 60%, at least about 70%, at least about 80%, at least about 90%, at least about 95%, or at least about 100% higher than the immunomodulatory imide compound alone or the binding moiety alone. In some aspects, the conjugate comprising an immunomodulatory imide compound, e.g., 5′-substituted isoindoline compound, and a binding moiety has in vitro anti-proliferative activity at least about 2 fold, at least about 3 fold, at least about 4 fold, at least about 5 fold, at least about 6 fold, at least about 7 fold, at least about 8 fold, at least about 9 fold, at least about 10 fold higher than the immunomodulatory imide compound alone or the binding moiety alone.

In some aspects, the conjugates described herein have in vitro anti-proliferative activity against a BT-474 breast cancer cell line, e.g., higher anti-proliferative activity against a BT-474 breast cancer cell line, compared to the immunomodulatory imide compound alone or the binding moiety alone. In some aspects, the conjugates described herein have in vitro anti-proliferative activity against the HL-60 acute myeloid leukemia cell line, e.g., higher anti-proliferative activity against a HL-60 acute myeloid leukemia cell line, compared to the immunomodulatory imide compound alone or the binding moiety alone. In some aspects, the conjugates described herein have in vitro anti-proliferative activity against a Ramos non-Hodgkins lymphoma cell line, , e.g., higher anti-proliferative activity against a Ramos non-Hodgkins lymphoma cell line, compared to the immunomodulatory imide compound alone or the binding moiety alone. In some aspects, the conjugates described herein is capable of maintaining their anti-proliferative activity in the presence of human serum. The conjugates described herein can be used in the treatment of cancers.

### III.A. Linker

The immunomodulatory imide compound of the present disclosure can be linked to the binding moiety via a linker. As used herein, the term “linker” refers to any chemical moiety capable of connecting the binding moiety (Bm) to group X within the compounds of formula (I).

In certain aspects, the linker can contain a heterobifunctional group. In the present disclosure, the term “heterobifunctional group” refers to a chemical moiety that connects the linker of which it is a part to the binding moiety. Heterobifunctional groups are characterized as having different reactive groups at either end of the chemical moiety. Attachment to “Bm,” can be accomplished through chemical or enzymatic conjugation, or a combination of both. Chemical conjugation involves the controlled reaction of accessible amino acid residues on the surface of the binding moiety with a reaction handle on the heterobifunctional group. Examples of chemical conjugation include, but are not limited to, lysine amide coupling, cysteine coupling, and coupling via a non-natural amino acid incorporated by genetic engineering, wherein non-natural amino acid residues with a desired reaction handle are installed onto “Bm.” In enzymatic conjugation, an enzyme mediates the coupling of the linker with an accessible amino residue on the binding moiety. Examples of enzymatic conjugation include, but are not limited to, transpeptidation using sortase, transpeptidation using microbial transglutaminase, and N-glycan engineering. Chemical conjugation and enzymatic conjugation may also be used sequentially. For example, enzymatic conjugation can also be used for installing unique reaction handles on “Bm” to be utilized in subsequent chemical conjugation.

In some aspects, the heterobifunctional group is selected from:

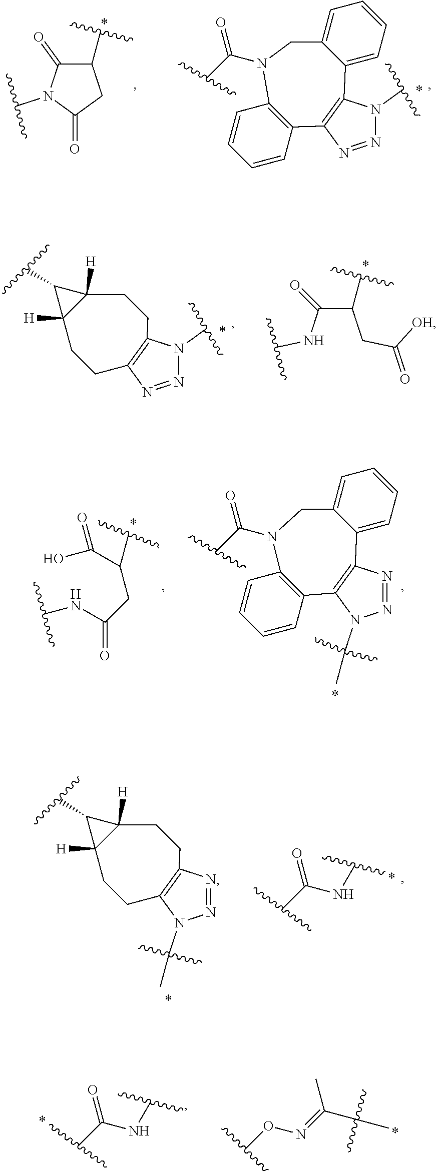

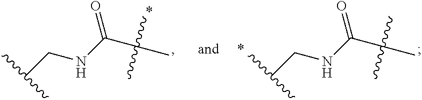 [SMILES: *CNC(*)=O ; *CNC(*)=O]

wherein
   [symbol] is the point of attachment to the remaining portion of the linker; and
   [symbol] the point of attachment to Bm.

In certain aspects, linker “L” is non-cleavable. As used here, the term “non-cleavable linker” is any chemical moiety that is capable of linking the binding moiety to the immunomodulatory imide compound in a stable, covalent manner and does not fall under the categories defined herein as “cleavable linkers”. Thus, non-cleavable linkers are substantially resistant to acid-induced cleavage, light-induced cleavage, bioreductive cleavage, peptidase-induced cleavage, esterase-induced cleavage, and disulfide bond cleavage. “Substantially resistant to cleavage” means that the chemical bond in the linker or adjoining the linker in at least 80%, preferably at least 85%, more preferably at least 90%, even more preferably at least 95%, and most preferably at least 99% of the conjugate population remains non-cleavable by an acid, a photolabile-cleaving agent, a bioreductive agent, a peptidase, an esterase, or a chemical or a physiological compound that cleaves the chemical bond (for example, a disulfide bond) in a cleavable linker, for within a few hours to several days of treatment with any of the agents described above. In certain aspects the linker is not susceptible to acid-induced cleavage, photo-induced cleavage, bioreductive cleavage, enzymatic cleavage, or the like, at conditions under which the immunomodulatory imide compound and/or binding moiety can remain active. A person of ordinary skill in the art would readily distinguish non-cleavable from cleavable linkers. ADC catabolites generated from non-cleavable linkers contain a residual amino acid from the antibody. These catabolites can exert unique and unexpected properties in the target cells to which they are delivered.

Examples of non-cleavable linkers include, but are not limited to, SMCC (succinimidyl 4-(N-maleimidomethyl)cyclohexane-1-carboxylate) linkers, succinimide thioether linkers, and linkers such as:

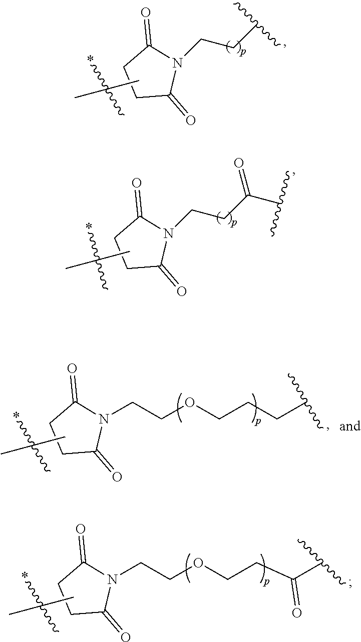

wherein:
p is an integer from 1 to 10;
[symbol] is the point of attachment to X; and
[symbol] is the point of attachment to the binding moiety.

In some aspects, the linker is:

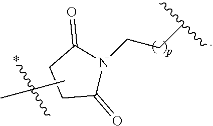

In some aspects, p is 5.

In some aspects, the linker is:

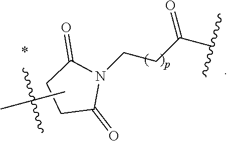

In some aspects, p is 5.

In certain aspects the linker can be cleavable. In some aspects, the linker can be susceptible to acid-induced cleavage, photo-induced cleavage, bioreductive cleavage, enzymatic cleavage, or the like, at conditions under which the immunomodulatory imide compound and/or binding moiety can remain active.

In some aspects, the cleavable linker can be cleaved enzymatically. In some aspects, the cleavable linker can be cleaved by a protease, peptidase, esterase, beta-gluroronidase, glycosidase, phosphodiesterase, phosphatase, pyrophosphatase, or lipase.

In some aspects, the cleavable linker can be cleaved by a protease. Examples of proteases include, but are not limited to, cathepsin B, VAGP tetrapeptide, and the like.

In certain aspects, the cleavable linker contains a peptide. In some aspects, the peptide is the site of cleavage of the linker, thereby facilitating release of the drug upon exposure to intracellular proteases, such as lysosomal enzymes. Peptides can be designed and optimized for enzymatic cleavage by a particular enzyme, for example, a tumor-associated protease, cathepsin B, C and D, or a plasmin protease. Examples of peptides having two amino acids include, but are not limited to, alanine-alanine (ala-ala), valine-alanine (val-ala), valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Examples of peptides having three amino acids include, but are not limited to, glycine-valine-citrulline (gly-val-cit), aspartic acid-valine-citrulline (asp-val-cit), alanine-alanine-asparagine (ala-ala-asn), alanine-phenylalanine-lysine (ala-phe-lys), glycine-glycine-phenylalanine (gly-gly-phe), and glycine-glycine-glycine (gly-gly-gly). Examples of peptides having four amino acids include, but are not limited to, glycine-glycine-valine-citrulline (gly-gly-val-cit) and glycine-glycine-phenylalanine-glycine (gly-gly-phe-gly). The amino acid combinations above can also be present in the reverse order (i.e., cit-val).

The peptides of the present disclosure can comprise L- or D-isomers of amino acid residues. The term “naturally-occurring amino acid” refer to Ala, Asp, Cys, Glu, Phe, Gly, His, He, Lys, Leu, Met, Asn, Pro, Gin, Arg, Ser, Thr, Val, Trp, Tyr, or citrulline. “D-” designates an amino acid having the “D” (dextrorotary) configuration, as opposed to the configuration in the naturally occurring (“L-”) amino acids. The amino acids described herein can be purchased commercially (Sigma Chemical Co., Advanced Chemtech) or synthesized using methods known in the art.

In certain aspects, the linker (“L”) is a protease cleavable linker selected from

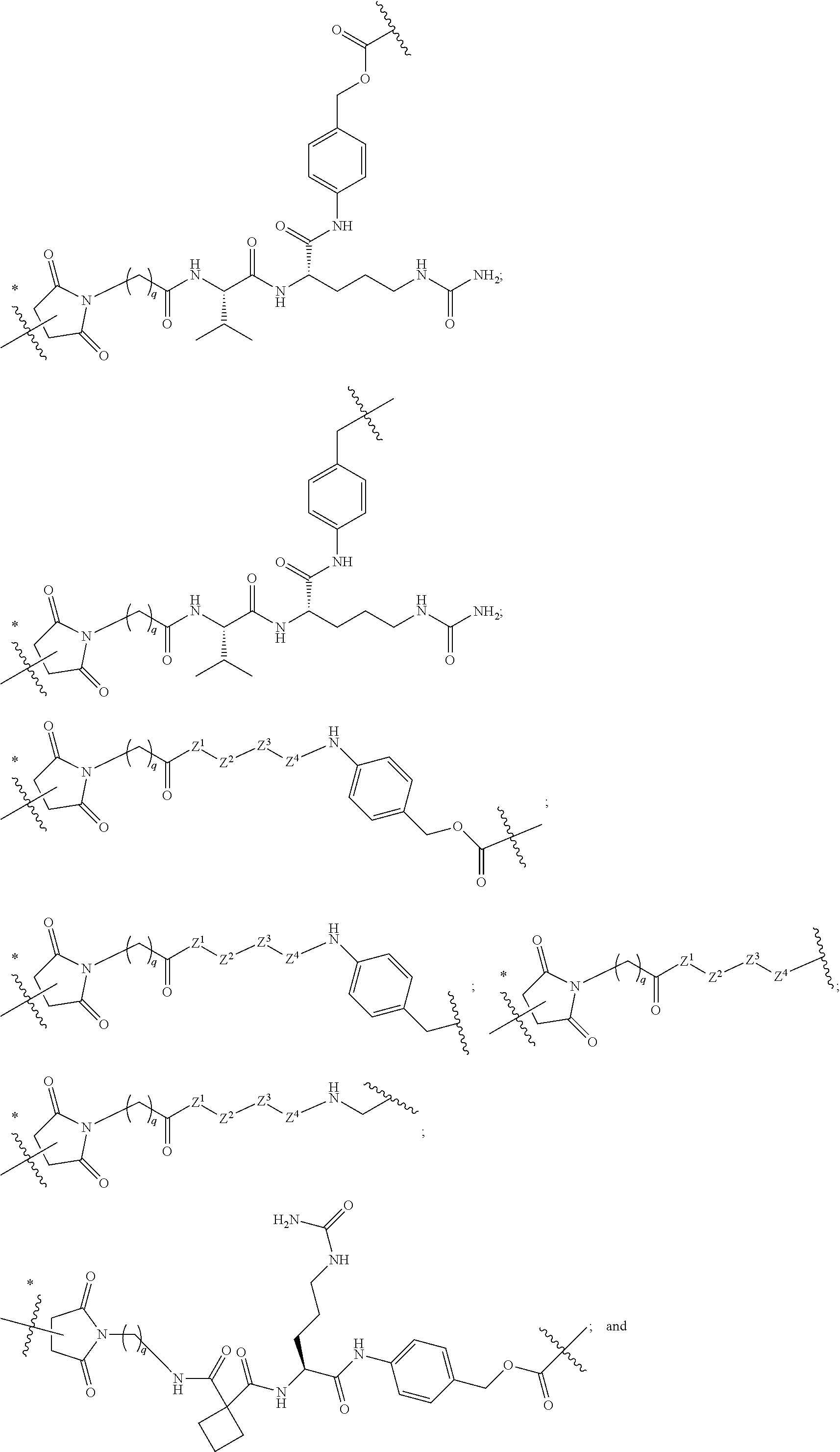

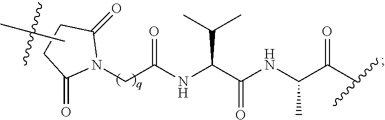

wherein:
   q is an integer from 2 to 10;
   Z¹, Z², Z³, and Z⁴ are each independently absent or a naturally-occurring amino acid residue in the L- or D-configuration, provided that at least two of Z¹, Z², Z³, and Z⁴ are amino acid residues;
   [symbol] is the point of attachment to X; and
   [symbol] is the point of attachment to the binding moiety.

In certain aspects, Z¹, Z², Z³, and Z⁴ are independently absent or selected from the group consisting of L-valine, D-valine, L-citrulline, D-citrulline, L-alanine, D-alanine, L-glutamine, D-glutaimine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, and Z⁴ are amino acid residues.

In some aspects, Z¹ is absent or glycine; Z² is absent or selected from L-glutamine, D-glutamine, L-glutamic acid, D-glutamic acid, L-aspartic acid, D-aspartic acid, L-alanine, D-alanine, and glycine; Z³ is selected from L-valine, D-valine, L-alanine, D-alanine, L-phenylalanine, D-phenylalanine, and glycine; and Z⁴ is selected from L-alanine, D-alanine, L-citrulline, D-citrulline, L-asparagine, D-asparagine, L-lysine, D-lysine, L-phenylalamine, D-phenylalanine, and glycine.

In some aspects, L is

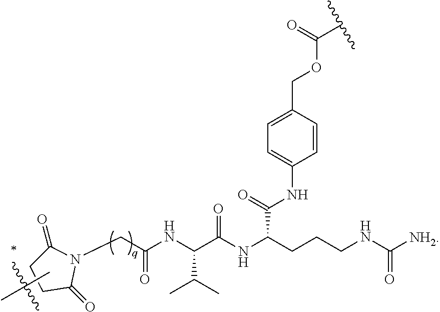

In some aspects, q is 5.

In certain aspects, L is a pyrophosphatase cleavable linker.

In some aspects, L is a pyrophosphatase cleavable linker which is:

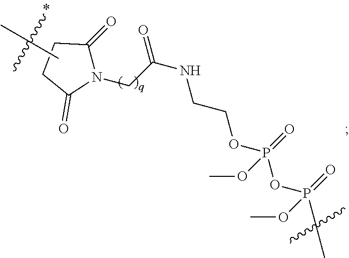

wherein:
   q is an integer from 2 to 10;
   [symbol] is the point of attachment to X; and
   [symbol] is the point of attachment to the binding moiety.

In certain aspects, L is a beta-glucoronidase cleavable linker.

In some aspects, L is a beta-glucoronidase cleavable linker which is:

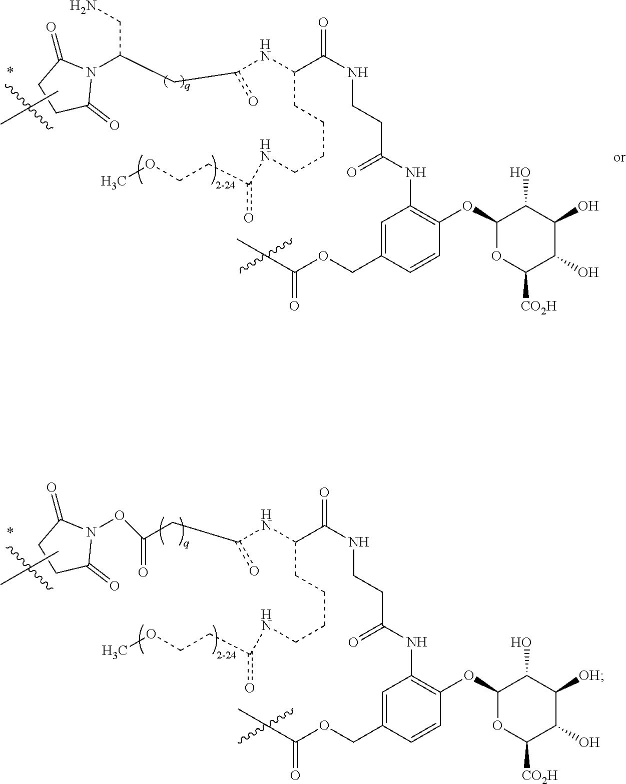

wherein:
   q is an integer from 2 to 10;
   ---- is absent or a bond;
   [symbol] is the point of attachment to X; and
   [symbol] is the point of attachment to the binding moiety.

In some aspects, the linker is bioreducible. Bioreducible linkers take advantage of the difference in reduction potential in the intracellular compartment versus plasma. Reduced glutathione presented in tumor cells' cytoplasma is up to 1000-fold higher than that present in normal cells' cytoplasma, and the tumor cells also contain enzymes which can contribute to reduction in cellular compartments. The linkers keep conjugates intact during systemic circulation, and are selectively cleaved by the high intracellular concentration of glutathione, releasing the active drugs at the tumor sites from the non-toxic prodrugs.

In some aspects, L is a bioreducible linker selected from:

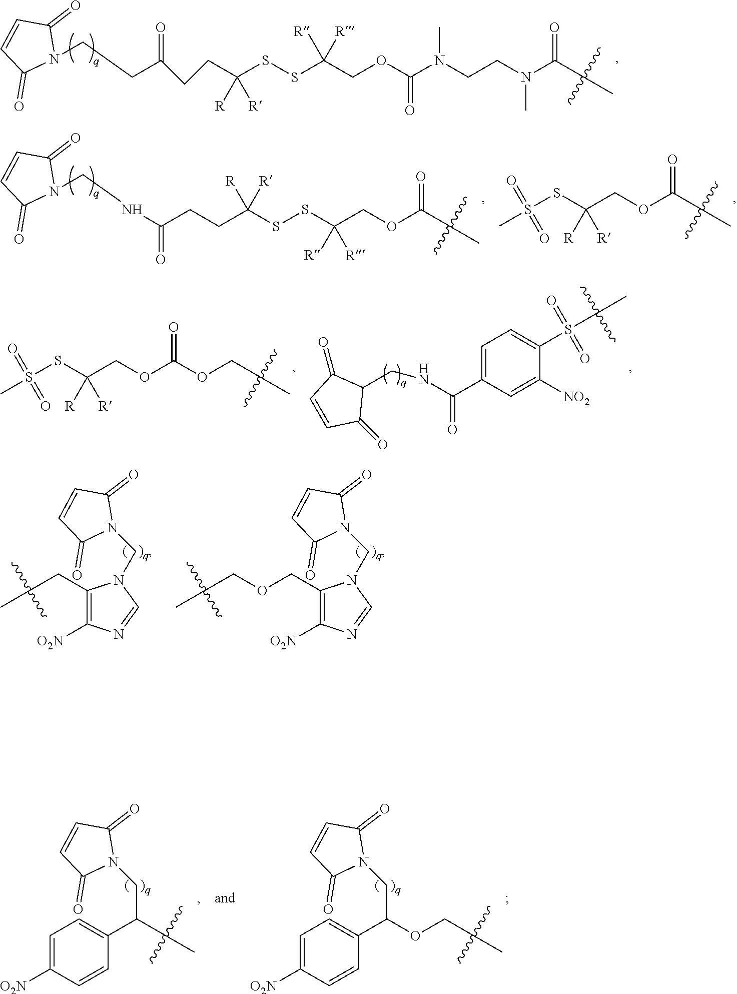

wherein:
   q is an integer from 2 to 10;
   R, R′, R″, and R′″ are each independently selected from hydrogen, C₁-C₆alkoxyC₁-C₆alkyl, (C₁-C₆)₂NC₁-C₆alkyl, and C₁-C₆alkyl or, two geminal R groups, together with the carbon atom to which they are attached, can form a cyclobutyl or cyclopropyl ring;
   [symbol] is the point of attachment to X; and
   [symbol] is the point of attachment to the binding moiety.

In certain aspects, the linker is acid cleavable. Acid-cleavable linkers are specifically designed to remain stable at the neutral pH of blood circulation, but undergo hydrolysis and release the cytotoxic drug in the acidic environment of the cellular compartments.

In some aspects, L is an acid cleavable linker selected from

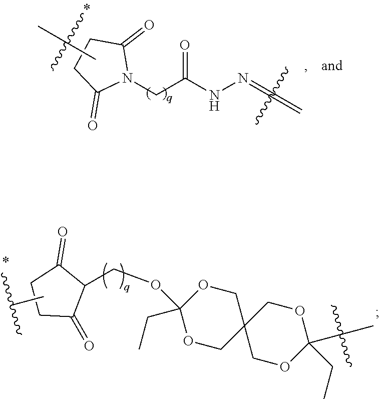

wherein:
   q is an integer from 2 to 10;
   [symbol] is the point of attachment to X; and
   [symbol] is the point of attachment to the binding moiety.

In certain aspects, L is wherein L is a click-to-release linker, where release of the immunomodulatory imide compound is chemically triggered by a tetrazine or related compound.

In some aspects, L is a click-to-release linker selected from

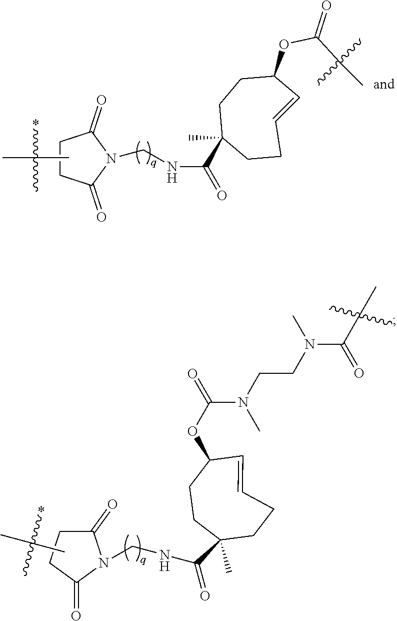

wherein:
   q is an integer from 2 to 10;
   [symbol] is the point of attachment to X; and
   [symbol] is the point of attachment to the binding moiety.

### III.B. Binding Moiety

The present disclosure provides immunomodulatory imide compound conjugated to binding moieties. The term “binding moiety,” as used herein, refers to any molecule that recognizes and binds to a cell surface marker or receptor. The binding moiety, in addition to targeting the immunomodulatory imide compound, e.g, 5′-substituted isoindoline compound, to a specific cell, tissue, or location, may also have certain therapeutic effect such as antiproliferative (cytostatic and/or cytotoxic) activity against a target cell or pathway. In certain aspects the binding moiety can comprise or can be engineered to comprise at least one chemically reactive group such as a carboxylic acid, amine, thiol, or chemically reactive amino acid moiety or side chain. In some aspects, the binding moiety can comprise a targeting moiety which binds or complexes with a cell surface molecule, such as a cell surface receptor or antigen, for a given target cell population. Following specific binding or complexing with the receptor, the cell is permissive for uptake of the targeting moiety or the immunomodulatory imide compound, e.g., 5′-substituted isoindoline, conjugate, which is then internalized into the cell.

In some aspects, group “Bm” can be a moiety that can specifically bind to a cell surface molecule. In some aspects, group “Bm” can be a peptide or a protein that binds to a cell surface receptor or antigen.

In certain aspects, group “Bm” can be an antibody, antibody fragment, or an antigen-binding fragment. An antibody is a protein generated by the immune system that is capable of recognizing and binding to a specific antigen. A target antigen generally has numerous binding sites, also called epitopes, recognized by CDRs on multiple antibodies. Each antibody that specifically binds to a different epitope has a different structure. Thus, one antigen may have more than one corresponding antibody. The term “antibody” herein is used in the broadest sense and specifically covers monoclonal antibodies, single domain antibodies, polyclonal antibodies, multispecific antibodies (e.g., bispecific antibodies), and antibody fragments, so long as they exhibit the desired biological activity. Antibodies may be murine, human, humanized, chimeric, or derived from other species.

Monoclonal antibodies that can be conjugated to the immunomodulatory imide compounds, e.g., 5′-substituted isoindoline compounds, are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture. These include, but are not limited to, the hybridoma technique, the human B cell hybridoma technique, and the EBV-hybridoma technique. Such antibodies may be of any immunoglobulin class including IgG, IgM, IgE, IgA, and IgD and any subclass thereof. The hybridoma producing the mAbs of use in this disclosure may be cultivated in vitro or in vivo.

Useful monoclonal antibodies include, but are not limited to, human monoclonal antibodies, humanized monoclonal antibodies, antibody fragments, or chimeric human-mouse (or other species) monoclonal antibodies. Human monoclonal antibodies may be made by any of numerous techniques known in the art.

The antibody can also be a bispecific antibody. Methods for making bispecific antibodies are known in the art. Traditional production of full-length bispecific antibodies is based on the coexpression of two immunoglobulin heavy chain-light chain pairs, where the two chains have different specificities. Because of the random assortment of immunoglobulin heavy and light chains, these hybridomas (quadromas) produce a potential mixture of 10 different antibody molecules, of which only one has the correct bispecific structure. Purification of the correct molecule, which is usually performed using affinity chromatography steps, is rather cumbersome, and the product yields are low.

According to a different approach, antibody variable domains with the desired binding specificities (antibody-antigen combining sites) are fused to immunoglobulin constant domain sequences. The fusion may be with an immunoglobulin heavy chain constant domain, comprising at least part of the hinge, C.sub.H2, and C.sub.H3 regions. The first heavy-chain constant region (C.sub.H1) may contain the site necessary for light chain binding, present in at least one of the fusions. Nucleic acids with sequences encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in aspects when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

Bispecific antibodies may have a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. Using such techniques, bispecific antibodies can be prepared for conjugation to the immunomodulatory imide compounds, e.g., 5′-substituted isoindoline compounds, in the treatment or prevention of disease as defined herein.

Hybrid or bifunctional antibodies can be derived either biologically, i.e., by cell fusion techniques, or chemically, especially with cross-linking agents or disulfide-bridge forming reagents, and may comprise whole antibodies or fragments thereof.

The antibody can be a functionally active fragment, derivative or analog of an antibody that immunospecifically binds to cancer cell antigens, viral antigens, or microbial antigens or other antibodies bound to tumor cells or matrix. In this regard, “functionally active” means that the fragment, derivative or analog is able to elicit anti-anti-idiotype antibodies that recognize the same antigen that the antibody from which the fragment, derivative or analog is derived recognized. Specifically, in an exemplary aspect the antigenicity of the idiotype of the immunoglobulin molecule can be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

Other useful antibodies include fragments of antibodies such as, but not limited to, F(ab′)2 fragments, which contain the variable region, the light chain constant region and the CH1 domain of the heavy chain can be produced by pepsin digestion of the antibody molecule, and Fab fragments, which can be generated by reducing the disulfide bridges of the F(ab′)2 fragments. Other useful antibodies are heavy chain and light chain dimers of antibodies, or any minimal fragment thereof such as Fvs or single chain antibodies (SCAs), or any other molecule with the same specificity as the antibody.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as those having a variable region derived from a murine monoclonal and human immunoglobulin constant regions. Humanized antibodies are antibody molecules from non-human species having one or more complementarity determining regions (CDRs) from the non-human species and a framework region from a human immunoglobulin molecule. Such chimeric and humanized monoclonal antibodies can be produced by recombinant DNA techniques known in the art.

Completely human antibodies can be produced using transgenic mice that are incapable of expressing endogenous immunoglobulin heavy and light chains genes, but which can express human heavy and light chain genes. The transgenic mice are immunized in the normal fashion with a selected antigen, e.g., all or a portion of a polypeptide of the disclosure. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies. Other human antibodies can be obtained commercially from, for example, Abgenix, Inc. (Freemont, Calif.) and Genpharm (San Jose, Calif.).

Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope. Human antibodies can also be produced using various techniques known in the art, including phage display libraries.

The antibody can be a fusion protein of an antibody, or a functionally active fragment thereof, for example in which the antibody is fused via a covalent bond (e.g., a peptide bond), at either the N-terminus or the C-terminus to an amino acid sequence of another protein (or portion thereof, such as at least 10, 20 or 50 amino acid portion of the protein) that is not the antibody. The antibody or fragment thereof may be covalently linked to the other protein at the N-terminus of the constant domain.

Antibodies include analogs and derivatives that are either modified, i.e., by the covalent attachment of any type of molecule as long as such covalent attachment permits the antibody to retain its antigen binding immunospecificity. For example, but not by way of limitation, the derivatives and analogs of the antibodies include those that have been further modified, e.g., by glycosylation, acetylation, pegylation, phosphorylation, amidation, derivatization by known protecting/blocking groups, proteolytic cleavage, linkage to a cellular antibody unit or other protein, etc. Any of numerous chemical modifications can be carried out by known techniques, including, but not limited to specific chemical cleavage, acetylation, formylation, metabolic synthesis in the presence of tunicamycin, etc. Additionally, the analog or derivative can contain one or more unnatural amino acids.

The antibodies in the conjugates can include antibodies having modifications (e.g., substitutions, deletions or additions) in amino acid residues that interact with Fc receptors. In particular, antibodies include antibodies having modifications in amino acid residues identified as involved in the interaction between the anti-Fc domain and the FcRn receptor. Antibodies immunospecific for a cancer cell antigen can be obtained commercially, for example, from Genentech (San Francisco, Calif.) or produced by any method known to one of skill in the art such as, e.g., chemical synthesis or recombinant expression techniques. The nucleotide sequence encoding antibodies immunospecific for a cancer cell antigen can be obtained, e.g., from the GenBank database or a database like it, the literature publications, or by routine cloning and sequencing.

In certain aspects, the antibody of the conjugates can be a monoclonal antibody, e.g. a murine monoclonal antibody, a chimeric antibody, or a humanized antibody. In some aspects, the antibody can be an antibody fragment, e.g. a Fab fragment.

Known antibodies for the treatment or prevention of cancer can be conjugated to the immunomodulatory imide compounds, e.g., 5′-substituted isoindoline compounds, described herein. Antibodies immunospecific for a cancer cell antigen can be obtained commercially or produced by any method known to one of skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing. Examples of antibodies available for the treatment of cancer include, but are not limited to, humanized anti-HER2 monoclonal antibody for the treatment of patients with metastatic breast cancer; RITUXAN® (rituximab; Genentech) which is a chimeric anti-CD20 monoclonal antibody for the treatment of patients with non-Hodgkin's lymphoma; OvaRex (oregovomab; AltaRex Corporation, MA) which is a murine antibody for the treatment of ovarian cancer; Panorex (edrecolomab, Glaxo Wellcome, NC) which is a murine IgG.sub.2a antibody for the treatment of colorectal cancer; Cetuximab Erbitux (cetuximab, Imclone Systems Inc., NY) which is an anti-EGFR IgG chimeric antibody for the treatment of epidermal growth factor positive cancers, such as head and neck cancer; Vitaxin (etaracizumab, MedImmune, Inc., MD) which is a humanized antibody for the treatment of sarcoma; Campath I/H (alemtuzumab, Leukosite, MA) which is a humanized IgG.sub.1 antibody for the treatment of chronic lymphocytic leukemia (CLL); Smart MI95 (Protein Design Labs, Inc., CA) which is a humanized anti-CD33 IgG antibody for the treatment of acute myeloid leukemia (AML); LymphoCide (epratuzumab, Immunomedics, Inc., NJ) which is a humanized anti-CD22 IgG antibody for the treatment of non-Hodgkin's lymphoma; Smart ID10 (Protein Design Labs, Inc., CA) which is a humanized anti-HLA-DR antibody for the treatment of non-Hodgkin's lymphoma; Oncolym (Techniclone, Inc., CA) which is a radiolabeled murine anti-HLA-Dr10 antibody for the treatment of non-Hodgkin's lymphoma; Allomune (BioTransplant, CA) which is a humanized anti-CD2 mAb for the treatment of Hodgkin's Disease or non-Hodgkin's lymphoma; Avastin (bevacizumab, Genentech, Inc., CA) which is an anti-VEGF humanized antibody for the treatment of lung and colorectal cancers; Epratuzamab (Immunomedics, Inc., NJ and Amgen, CA) which is an anti-CD22 antibody for the treatment of non-Hodgkin's lymphoma; and CEAcide (Immunomedics, NJ) which is a humanized anti-CEA antibody for the treatment of colorectal cancer.

Other antibodies useful for the conjugates include, but are not limited to, trastuzumab, gemtuzumab, pertuzumab, obinutuzumab, ofatumumab, olaratumab, ontuximab, isatuximab, Sacituzumab, U3-1784, daratumumab, STI-6129, lintuzumab, huMy9-6, balantamab, indatuximab, dinutuximab, anti-CD38 A2 antibody, buAT15/3 H3s antibody, ibritumomab, tositumomab, panitumumab, tremelimumab, ticilimumab, catumaxomab, and veltuzumab. In certain aspects, the antibody is selected from the group consisting of rituximab, trastuzumab, pertuzumab, huMy9-6, lintuzumab, and gemtuzumab.

Other antibodies useful for the conjugates include, but are not limited to, antibodies against the following antigens: CA125 (ovarian), CA15-3 (carcinomas), CA19-9 (carcinomas), L6 (carcinomas), Lewis Y (carcinomas), Lewis X (carcinomas), alpha fetoprotein (carcinomas), CA 242 (colorectal), placental alkaline phosphatase (carcinomas), prostate specific antigen (prostate), prostatic acid phosphatase (prostate), epidermal growth factor (carcinomas), MAGE-1 (carcinomas), MAGE-2 (carcinomas), MAGE-3 (carcinomas), MAGE-4 (carcinomas), anti-transferrin receptor (carcinomas), p97 (melanoma), MUC1-KLH (breast cancer), CEA (colorectal), gp100 (melanoma), MART1 (melanoma), PSA (prostate), IL-2 receptor (T-cell leukemia and lymphomas), CD20 (non-Hodgkin's lymphoma), CD52 (leukemia), CD33 (leukemia), CD22 (lymphoma), human chorionic gonadotropin (carcinoma), CD38 (multiple myeloma), CD40 (lymphoma), mucin (carcinomas), P21 (carcinomas), MPG (melanoma), and Neu oncogene product (carcinomas). Some specific, useful antibodies include, but are not limited to, BR96 mAb (Trail, P. A., et al Science (1993) 261, 212-215), BR64 (Trail, P A, et al Cancer Research (1997) 57, 100-105), mAbs against the CD40 antigen, such as S2C6 mAb (Francisco, J. A., et al Cancer Res. (2000) 60:3225-3231), mAbs against the CD70 antigen, such as 1F6 mAb, and mAbs against the CD30 antigen, such as AC10. Many other internalizing antibodies that bind to tumor associated antigens can be used and have been reviewed.

Other antigens that the present conjugates can bind to include, but are not limited to, 5T4, ACE, ADRB3, AKAP-4, ALK, Androgen receptor, AOC3, APP, Axin1, AXL, B7H3, B7-H4, BCL2, BCMA, bcr-abl, BORIS, BST2, C242, C4.4a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudine 3, CLDN6, CLEC12A, CLL-1, cll3, c-MET, Cripto protein, CS1, CTLA-4, CXCR2, CXORF61, Cyclin B1, CYP1B1, Cadherin-3, Cadherin-6, DLL3, E7, EDNRB, EFNA4, EGFR, EGFRvIII, ELF2M, EMR2, ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GloboH, GM3, GPC1, GPC2, GPC3, gplOO, GPNMB, GPR20, GPRC5D, GUCY2C, HAVCR1, HER2, HER3, HGF, HMI.24, HMWMAA, HPV E6, hTERT, human telomerase reverse transcriptase, ICAM, ICOS-L, IFN-α, IFN-γ, IGF-I receptor, IGLL1, IL-2 receptor, IL-4 receptor, IL-13Ra2, IL-l lRa, IL-1, IL-12, IL-23, IL-13, IL-22, IL-4, IL-5, IL-6, interferon receptor, integrins (including α4, αvβ3, αvβ5, αvβ6, α1β4, α4β1, α4β7, α5β1, α6β4, αIIbβ3 integrins), Integrin alphaV, intestinal carboxyl esterase, KIT, LAGE-la, LAIR1, LAMP-1, LCK, Legumain, LewisY, LFA-1(CD11a), L-selectin(CD62L), LILRA2, LIV-1, LMP2, LRRC15, LY6E, LY6K, LY75, MAD-CT-1, MAD-CT-2, MAGE Al, MelanA/MARTl, Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2, OY-TES1, p53, p53 mutant, PANX3, PAP, PAX3, PAX5, p-CAD, PCTA-1/Galectin 8, PD-L1, PD-L2, PDGFR, PDGFR-beta, phosphatidylserine, PIK3CA, PLAC1, Polysialic acid, Prostase, prostatic carcinoma cell, prostein, Pseudomonas aeruginosa, rabies, survivin and telomerase, PRSS21, PSCA, PSMA, PTK7, RAGE-1, RANKL, Ras mutant, respiratory syncytial virus, Rhesus factor, RhoC, RON, ROR1, ROR2, RU1, RU2, sarcoma translocation breakpoints, SART3, SLAMF7, SLC44A4, sLe, SLITRK6, sperm protein 17, sphingosine-1-phosphate, SSEA-4, SSX2, STEAP1, TAG72, TARP, TCRβ, TEM1/CD248, TEM7R, tenascin C, TF, TGF-1, TGF-β2, TNF-α, TGS5, Tie 2, TIM-1, Tn Ag, TRAC, TRAIL-R1, TRAIL-R2, TROP-2, TRP-2, TRPV1, TSHR, tumor antigen CTAA16.88, tyrosinase, UPK2, VEGF, VEGFR1, VEGFR2, vimentin, WTl, and/or XAGE1.

Antibodies that bind to antigens associated with antigen presenting cells such as CD40, OX40L, Endoglin, DEC-205, 4-1BBL, CD36, CD36, CD204, MARCO, DC-SIGN, CLEC9A, CLEC5A, Dectin 2, CLEC10A, CD206, CD64, CD32A, CD1A, HVEM, CD32B, PD-L1, BDCA-2, XCR-1, and CCR2 can also be conjugated to the immunomodulatory imide compound.

Antibodies of a conjugate can bind to both a receptor or a receptor complex expressed on an activated lymphocyte. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein. Non-limiting examples of suitable immunoglobulin superfamily members are CD2, CD3, CD4, CD8, CD 19, CD22, CD28, CD79, CD90, CD 152/CTLA-4, PD-1, and ICOS. Non-limiting examples of suitable TNF receptor superfamily members are CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, and APO-3. Non-limiting examples of suitable integrins are CD 11a, CD11b, CD 11c, CD18, CD29, CD41, CD49a, CD49b, CD49c, CD49d, CD49e, CD49f, CD 103, and CD 104. Non-limiting examples of suitable lectins are C-type, S-type, and I-type lectin.

In some aspects, the antibodies that can are useful for the present disclosure include, but are not limited to, 3F8, 8H9, abagovomab, abciximab (REOPRO®), abituzumab, abrezekimab, abrilumab, actoxumab, adalimumab (HUMIRA®), adecatumumab, aducanumab, afasevikumab, afelimomab, afutuzumab, alacizumab, ALD518, alemtuzumab (CAMPATH®), alirocumab (PRALUENT®), altumomab, amatuximab, anatumomab, andecaliximab, anetumab, anifrolumab, anrukinzumab, apolizumab, aprutumab, arcitumomab (CEA-SCAN®), ascrinvacumab, aselizumab, atidortoxumab, atlizumab (tocilizumab, ACTEMRA®, ROACTEMRA®), atezolizumab (TECENTRIQ®), atinumab, atorolimumab, avelumab (Bavencio), azintuxizumab, balantamab, bapineuzumab, basiliximab (SIMULECT®), bavituximab, BCD-100, bectumomab (LYMPHOSCAN®), begelomab, belantamab, belimumab (BENLYSTA®), bemarituzumab, benralizumab (FASENRA®), bermekimab, bersanlimab, bertilimumab, besilesomab (SCINITIMUN®), bevacizumab (AVASTIN®), bezlotoxumab (ZINPLAVA®), biciromab (FIBRISCINT), bimagrumab, bimekizumab, birtamimab, bivatuzumab, bleselumab, blinatumomab, blontuvetmab, blosozumab, bococizumab, brazikumab, brentuximab, briakinumab, brodalumab (SILIQ™), brolucizumab (BEOVU® brontictuzumab, burosumab (CRYSVITA®), cabiralizumab, caplacizumab (CABLIVI®), camidanlumab, camrelizumab, canakinumab (ILARIS®), cantuzumab, capromab, carlumab, carotuximab, catumaxomab (REMOVAB®), cBR96, CC49, cedelizumab, cemiplimab (LIBTAYO®), cergutuzumab, certrelimab, certolizumab, cetuximab (ERBITUX®), cibisatamab, cirmtuzumab, citatuzumab, cixutumumab, clazakizumab, clenoliximab, clivatuzumab, codrituzumab, cofetuzumab, coltuximab, conatumumab, concizumab, cosfroviximab, CR6261, crenezumab, crizanlizumab (ADAKVEO®), crotedumab, cusatuzumab, dacetuzumab, daclizumab (ZINBRYTA®), dalotuzumab, dapirolizumab, daratumumab (DARZALEX®), dectrekumab, demcizumab, denintuzumab, denosumab (PROLIA®), depatuxizumab, derlotuximab, detumomab, dezamizumab, dinutuximab (UNITUXIN®), diridavumab, domagrozumab, dostarlimab, dorlimomab, dorlixizumab, drozitumab, DS-8201, duligotuzumab, dupilumab (DUPIXENT®), durvalumab (IMFINZI®), dusigitumab, ecromeximab, eculizumab (SOLIRIS®), edobacomab, edrecolomab (PANOREX®), efalizumab (RAPTIVA®), efungumab (MYCOGRAB®), eldelumab, elezanumab, elgemtumab, elotuzumab (EMPLICITI®), elsilimomab, emactuzumab emapalumab (GAMIFANT®), emibetuzumab, emicizumab (HEMLIBRA®), enapotamab, enavatuzumab, enfortumab (PADCEV®), enlimomab, enoblituzumab, enokizumab, enoticumab, ensituximab, epitumomab, eptinezumab (VYEPTI® epratuzumab, erenumab (AIMOVIG®), erlizumab, ertumaxomab (REXOMUN®), etaracizumab (ABEGRIN®), etigilimab, etrolizumab, evinacumab, evolocumab (REPATHA®), exbivirumab, fanolesomab (NEUTROSPEC®), faralimomab, faricimab, farletuzumab, fasinumab, FBTA05, felvizumab, fezakinumab, fibatuzumab, ficlatuzumab, figitumumab, firivumab, flanvotumab, fletikumab, flotetuzumab, fontolizumab (HUZAF®), foralumab, foravirumab, fremanezumab (AJOVY®), fresolimumab, frovocimab, frunevetmab, fulranumab, futuximab, galcanezumab (EMGALITY®), galiximab, gancotamab, ganitumab, gantenerumab, gavilimomab, gedivumab, gemtuzumab, gevokizumab, gilvetmab, gimsilumab, girentuximab, glembatumumab, golimumab (SIMPONI®), gomiliximab, guselkumab (TREMFYA®), huMy9-6, ianalumab, ibalizumab (TROGARZO®), IBI308, ibritumomab, icrucumab, idarucizumab (PRAXBIND®), ifabotuzumab, igovomab (INDIMACIS-125), iladatuzumab, IMAB362, imalumab, imaprelimab, imeiromab (MYOSCINT®), imgatuzumab, inclacumab, indatuximab, indusatumab, inebilizumab, infliximab (REMICADE®), intetumumab, inolimomab, inotuzumab, iomab-B, ipilimumab, iratumumab, isatuximab (SARCLISA®), iscalimab, istiratumab, itolizumab, ixekizumab (TALTZ®), keliximab, labetuzumab (CEA-CIDE™), lacnotuzumab, ladiratuzumab, lampalizumab, lanadelumab (TAKHZYRO®), landogrozumab, laprituximab, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab, ligelizumab, lilotomab, lintuzumab, lirilumab, lodelcizumab, lokivetmab, loncastuximab, lorvotuzumab, losatuxizumab, lucatumumab, lulizumab, lumiliximab, lumretuzumab, lupartumab, lutikizumab, mapatumumab, margetuximab, marstacimab, maslimomab, matuzumab, mavrilimumab, mepolizumab (NUCALA®), metelimumab, milatuzumab, minretumomab, mirikizumab, mirvetuximab, mitumomab, modotuximab, molalizumab, mogamulizumab (POTELIGEO®), morolimumab, mosunetuzumab, motavizumab (NUMAX®), moxetumomab (LUMOXITI®), muromonab-CD3 (ORTHOCLONE OKT3®), nacolomab, namilumab, naptumomab, naratuximab, narnatumab, natalizumab (TYSABRI®), navicixizumab, navivumab, naxitamab, nebacumab, necitumumab (PORTRAZZA®), nemolizumab, NEOD001, nerelimomab, nesvacumab, netakimab, nimotuzumab (THERACIM®), nirsevimab, nivolumab, nofetumomab, obiltoxaximab (ANTHIM®), obinutuzumab, ocaratuzumab, ocrelizumab (OCREVUS®), odulimomab, ofatumumab (ARZERRA®), olaratumab (LARTRUVO®), oleclumab, olendalizumab, olokizumab, omalizumab (XOLAIR®), omburtamab, OMS721, onartuzumab, ontecizumab, ontuxizumab, onvatilimab, opicinumab, oportuzumab, oregovomab (OVAREX), orticumab, otelixizumab, otilimab, otlertuzumab, oxelumab, ozanezumab, ozogamicin, ozoralizumab, pagibaximab, palivizumab (SYNAGIS®), pamrevlumab, panitumumab (VECTIBIX®), pankomab, panobacumab, parsatuzumab, pascolizumab, pasotuxizumab, pateclizumab, patritumab, PDR001, pembrolizumab, pemtumomab (THERAGYN®), perakizumab, pertuzumab (OMNITARG®), pexelizumab, pidilizumab, pinatuzumab, pintumomab, placulumab, polatuzumab (Polivy), prezalumab, plozalizumab, pogalizumab, ponezumab, porgaviximab, prasinezumab, prezalizumab, priliximab, pritoxaximab, pritumumab, PRO 140, quilizumab, racotumomab, radretumab, rafivirumab, ralpancizumab, ramucirumab, ranevetmab, ranibizumab (LUCENTIS®), ravagalimab, ravulizumab (ULTOMIRIS®), raxibacumab, refanezumab, regavirumab, REGN-EB3, renatlimab, remtolumab, reslizumab (CINQAIR®), rilotumumab, rinucumab, risankizumab (SKYRIZI®), rituximab (RITUXAN®), rivabazumab, rmab, robatumumab, roledumab, romilkimab, romosozumab (EVENITY®), rontalizumab, rosmantuzumab, rovalpituzumab, rovelizumab (LEUKARREST®), rozanolixizumab, ruplizumab (ANTOVA), SA237, sacituzumab, samalizumab, samrotamab, sarilumab (KEVZARA®), satralizumab, satumomab pendetide, secukinumab (COSENTYX®), selicrelumab, seribantumab, setoxaximab, setrusumab, sevirumab, SGN-CD19A, SHP647, sibrotuzumab, sifalimumab, siltuximab, simtuzumab, siplizumab, sirtratumab, sirukumab, sofituzumab, solanezumab, solitomab, sonepcizumab, sontuzumab, spartalizumab, stamulumab, STI-6129, sulesomab (LEUKOSCAN®), suptavumab, sutimlimab, suvizumab, suvratoxumab, tabalumab, tacatuzumab (AFP-CIDE®), tadocizumab, talacotuzumab, talizumab, tamtuvetmab, tanezumab, taplitumomab paptox, tarextumab, tavolimab, tefibazumab (AUREXIS®), telimomab, telisotuzumab, tesidolumab, tetraxetan, tetulomab, tenatumomab, teneliximab, teprotumumab (TEPEZZA®), teplizumab, tezepelumab, TGN1412, tibulizumab, ticilimumab (TREMELIMUMAB®), tigatuzumab, timigutuzumab, timolumab, tiragolumab, tiragotumab, tislelizumab, tisotumab, tiuxetan, tildrakizumab (ILUMYA®), TNX-650, tocilizumab (atlizumab, ACTEMRA®), tomuzotuximab, toralizumab, tosatoxumab, tositumomab (BEXXAR®), tovetumab, tralokinumab, trastuzumab (HERCEPTIN®), TRBS07, tregalizumab, tremelimumab, trevogrumab, tucotuzumab, tuvirumab, urtoxazumab, ustekinumab (STELERA®), ublituximab, ulocuplumab, urelumab, utomilumab, vadastuximab, vanalimab, vandortuzumab, vantictumab, vanucizumab, vapaliximab, varisacumab, varlilumab, vatelizumab, vedolizumab, veltuzumab, vepalimomab, vesencumab, visilizumab (NUVION®), vobarilizumab, volociximab (HUMASPECT®), vonlerolizumab, vopratelimab, vorsetuzumab, votumumab, vunakizumab, xentuzumab, XMAB-5574, zalutumumab (HuMEX-EGFr), zanolimumab (HuMAX-CD4), zatuximab, zenocutuzumab, ziralimumab, zolbetuximab or zolimomab.

An antibody “which binds” a molecular target or an antigen of interest is one capable of binding that antigen with sufficient affinity such that the antibody is useful in targeting a cell expressing the antigen.

In the present disclosure, group “Bm” can be conjugated to more than one immunomodulatory imide compounds, e.g., 5′-substituted isoindoline compounds. In some aspects, “Bm” can be conjugated to from 1 to 10 immunomodulatory imide compounds, e.g., 5′-substituted isoindoline compounds. In some aspects, “Bm” can be conjugated to from 1 to 9 immunomodulatory imide compounds, e.g., 5′-substituted isoindoline compounds. In some aspects, “Bm” can be conjugated to from 1 to 8 immunomodulatory imide compounds, e.g., 5′-substituted isoindoline compounds. In some aspects, “Bm” can be conjugated to 1, 2, 3, 4, 5, 6, 7, 8, 9, or 10 immunomodulatory imide compounds, e.g., 5′-substituted isoindoline compounds. In some aspects, “Bm” can be conjugated to 7 or 8 immunomodulatory imide compounds, e.g., 5′-substituted isoindoline compounds. In some aspects, “Bm” is conjugated to 5 immunomodulatory imide compounds, e.g., 5′-substituted isoindoline compounds. In some aspects, “Bm” is conjugated to 6 immunomodulatory imide compounds, e.g., 5′-substituted isoindoline compounds. In some aspects, “Bm” is conjugated to 7 immunomodulatory imide compounds, e.g., 5′-substituted isoindoline compounds. In some aspects, “Bm” is conjugated to 8 immunomodulatory imide compounds, e.g., 5′-substituted isoindoline compounds. In some aspects, “Bm” is conjugated to 9 immunomodulatory imide compounds, e.g., 5′-substituted isoindoline compounds.

### IV. Immunomodulatory Imide Compounds

In certain aspects, the immunomodulatory imide compound is Formula (a):

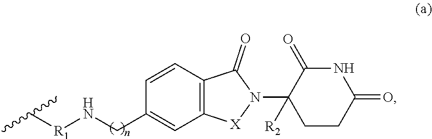

or a pharmaceutically acceptable salt, solvate, or stereoisomer thereof, wherein:
n is 0 or 1;
X is CH₂, C═O, or C═S;
[symbol] is the point of attachment to L;
R₁ is:
a) —(CH₂)ₘR₃ or —CO(CH₂)ₘR₃, wherein
   m is 0, 1, 2, or 3; and
   R₃ is 5-10 membered aryl or heteroaryl, optionally substituted with one or more halogen;
b) —C═YR₄, wherein
   Y is O or S; and
   R₄ is:
   (C₀-C₁₀)alkyl-(5 to 10 membered heteroaryl or heterocycle), said heteroaryl or heterocycle optionally substituted with one or more of (C₁-C₆)alkyl, halogen, oxo, (C₁-C₆)alkoxy, or —Z—(C₁-C₆)alkyl, wherein Z is S or SO₂, and wherein said (C₁-C₆)alkyl may be optionally substituted with one or more halogen;
   (C₀-C₁₀)alkyl-(5 to 10 membered aryl), said aryl optionally substituted with one or more of: halogen; (C₁-C₆)alkoxy, itself optionally substituted with one or more halogen; (C₁-C₆)alkyl, itself optionally substituted with one or more halogen; or —Z—(C₁-C₆)alkyl, wherein Z is S or SO₂, and wherein said (C₁-C₆)alkyl may be optionally substituted with one or more halogen; or (C₁-C₆)alkyl-CO—O—R₁₂, wherein R₁₂ is H or (C₁-C₆)alkyl; or
c) —C═ZNHR₆, wherein
   Z is O or S; and
   R₆ is:
   5 to 10 membered aryl or heteroaryl, optionally substituted with one or more of: halogen; cyano; (C₁-C₆)alkylenedioxy; (C₁-C₅)alkoxy, itself optionally substituted with one or more halogen; (C₁-C₆)alkyl, itself optionally substituted with one or more halogen; or (C₁-C₆)alkylthio, itself optionally substituted with one or more halogen; and
R₂ is H or (C₁-C₆)alkyl.

In certain aspects, the structure of the immunomodulatory imide compound of Formula (a) is that:
n is 0 or 1;
X is CH₂ or C═O;
R₁ is —(CH₂)ₘR₃, wherein
   m is 0, 1, 2, or 3; and
   R₃ is 5-10 membered aryl or heteroaryl, optionally substituted with one or more halogen.

In certain aspects, the structure of the immunomodulatory imide compound of Formula (a) is that:
n is 1;
X is CH₂ or C═O;
R₁ is:
   —C═OR₄ or —C═SR₄, wherein
      R₄ is:
      (C₀-C₁₀)alkyl-(5 to 10 membered heteroaryl or heterocycle), said heteroaryl or
      heterocycle optionally substituted with one or more of (C₁-C₆)alkyl, halogen, oxo, (C₁-C₆)alkoxy, or —Z—(C₁-C₆)alkyl, wherein Z is S or SO₂, and wherein said (C₁-C₆)alkyl may be optionally substituted with one or more halogen;
      (C₀-C₁₀)alkyl-(5 to 10 membered aryl), said aryl optionally substituted with one or more of: halogen; (C₁-C₆)alkoxy, itself optionally substituted with one or
      more halogen; (C₁-C₆)alkyl, itself optionally substituted with one or more halogen; or —Z—(C₁-C₆)alkyl, wherein Z is S or SO₂, and wherein said (C₁-C₆)alkyl may be optionally substituted with one or more halogen; or (C₁-C₆)alkyl-CO—O—R₁₂, wherein R₁₂ is H or (C₁-C₆)alkyl.

In certain aspects, the structure of the immunomodulatory imide compound of Formula (a) is that:
n is 1;
X is CH₂ or C═O;
R₁ is:
   —C═ONHR₆ or —C═OSHR₆, wherein
      R₆ is:
      5 to 10 membered aryl or heteroaryl, optionally substituted with one or more of: halogen; cyano; (C₁-C₆)alkylenedioxy; (C₁-C₅)alkoxy, itself optionally substituted with one or more halogen; (C₁-C₆)alkyl, itself optionally substituted with one or more halogen; or (C₁-C₆)alkylthio, itself optionally substituted with one or more halogen.

In certain aspects, the structure of the immunomodulatory imide compound of Formula (a) is that:
n is 1;
X is CH₂ or C═O;
R₁ is:
   —C═OR₄, wherein
   R₄ is:
   (C₀-C₁₀)alkyl-(5 to 10 membered heteroaryl or heterocycle), said heteroaryl or heterocycle optionally substituted with one or more of (C₁-C₆)alkyl, halogen, oxo, (C₁-C₆)alkoxy, or —Z—(C₁-C₆)alkyl, wherein Z is S or SO₂, and wherein said (C₁-C₆)alkyl may be optionally substituted with one or more halogen;
   (C₀-C₁₀)alkyl-(5 to 10 membered aryl), said aryl optionally substituted with one or more of: halogen; (C₁-C₆)alkoxy, itself optionally substituted with one or more halogen; (C₁-C₆)alkyl, itself optionally substituted with one or more halogen; or —Z—(C₁-C₆)alkyl, wherein Z is S or SO₂, and wherein said (C₁-C₆)alkyl may be optionally substituted with one or more halogen; or (C₁-C₆)alkyl-CO—O—R₁₂, wherein R₁₂ is H or (C₁-C₆)alkyl.

In certain aspects, the structure of the immunomodulatory imide compound of Formula (a) is

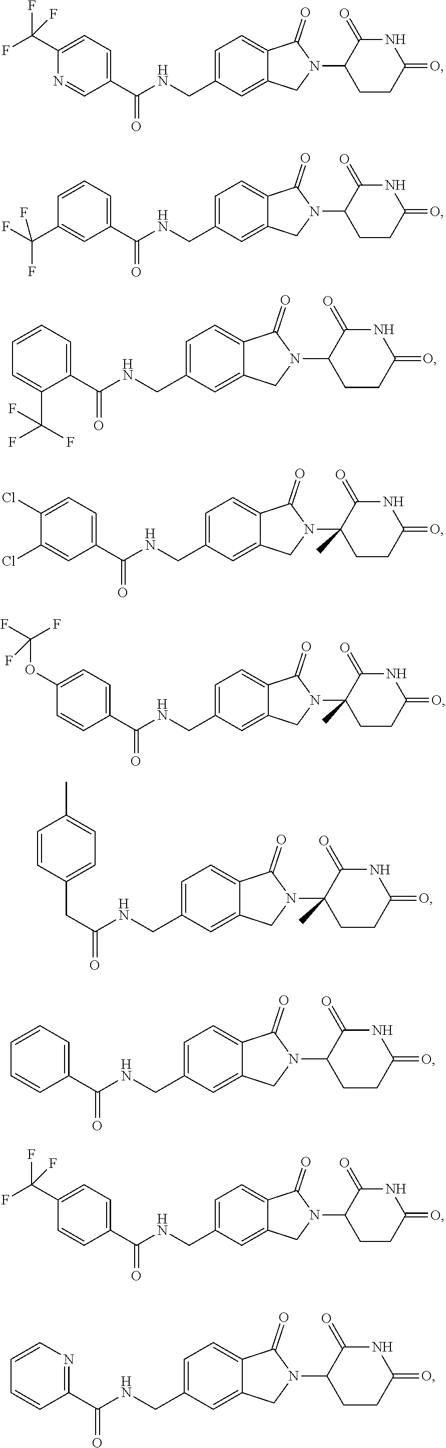

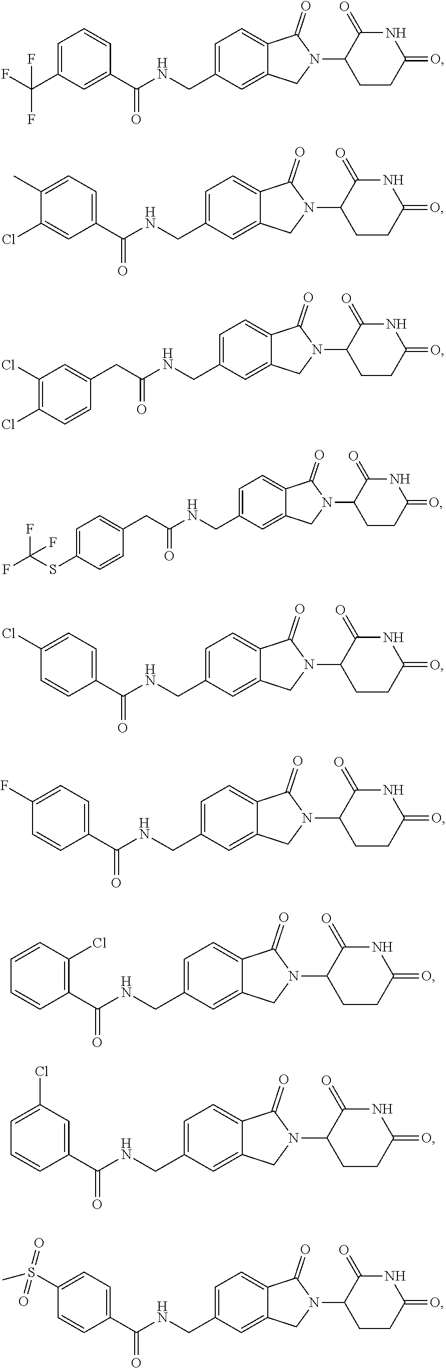

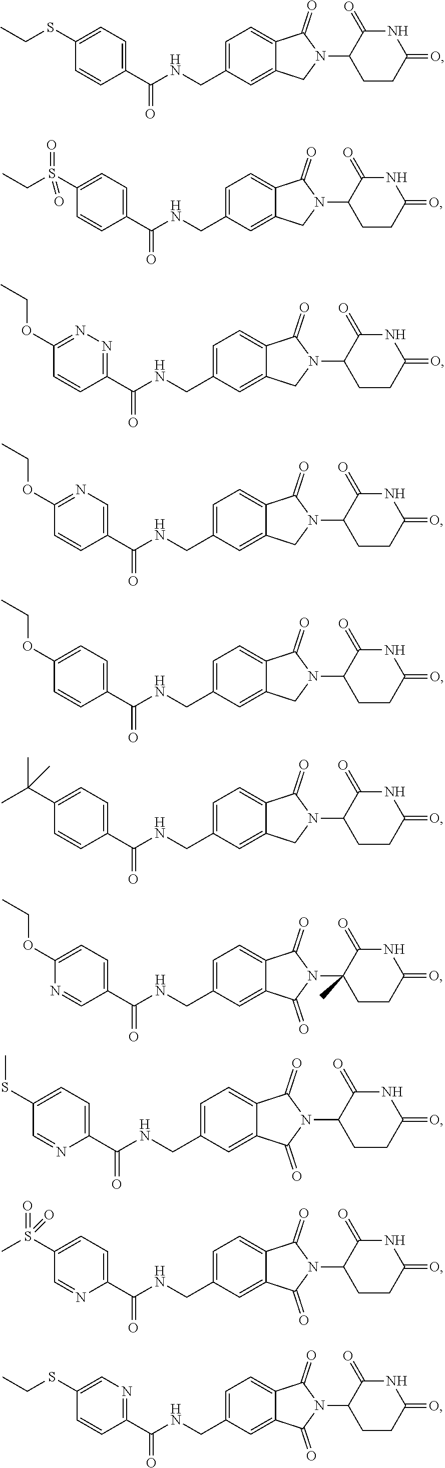

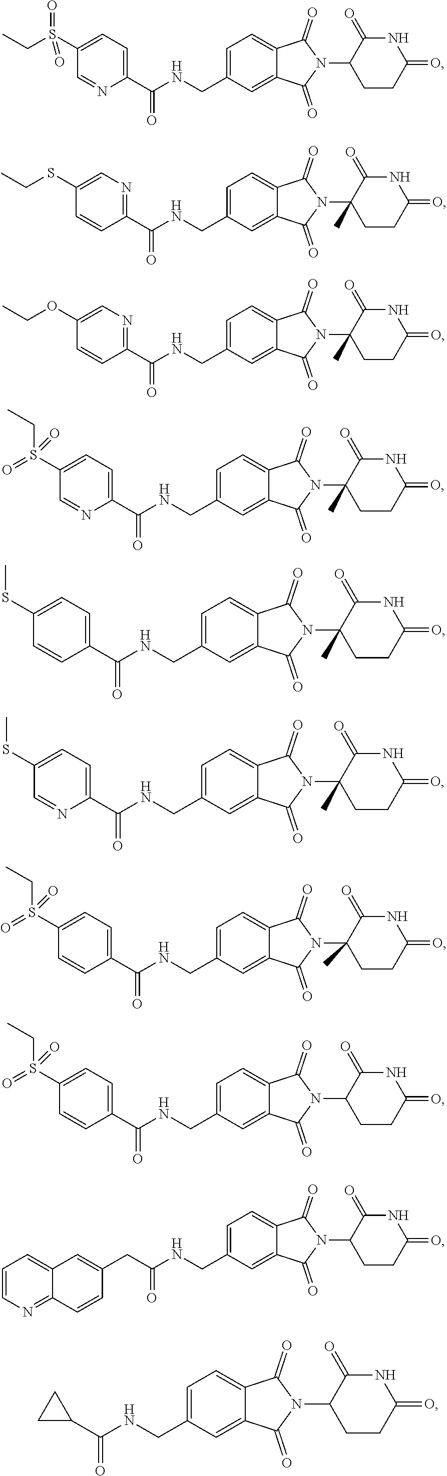

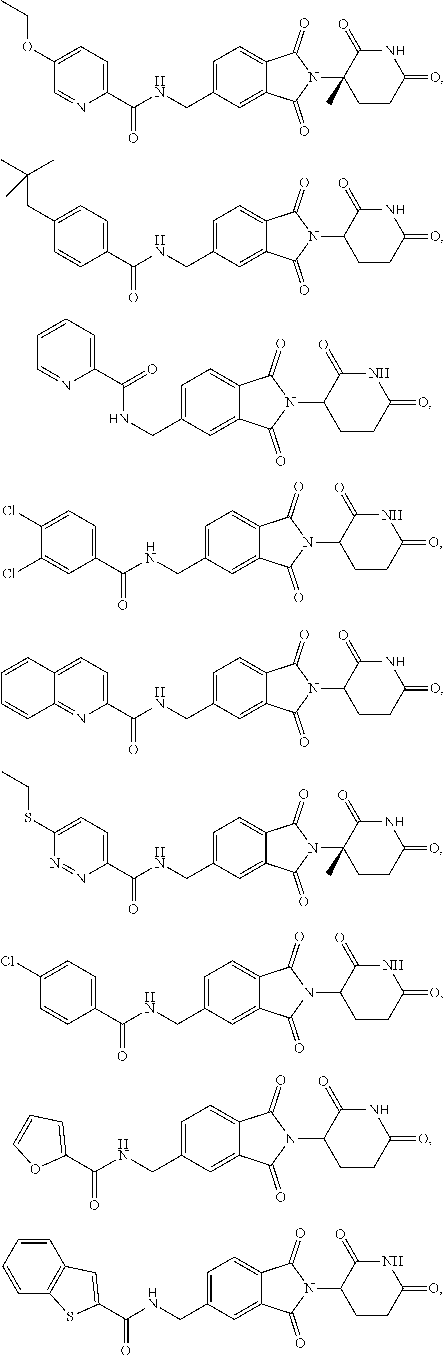

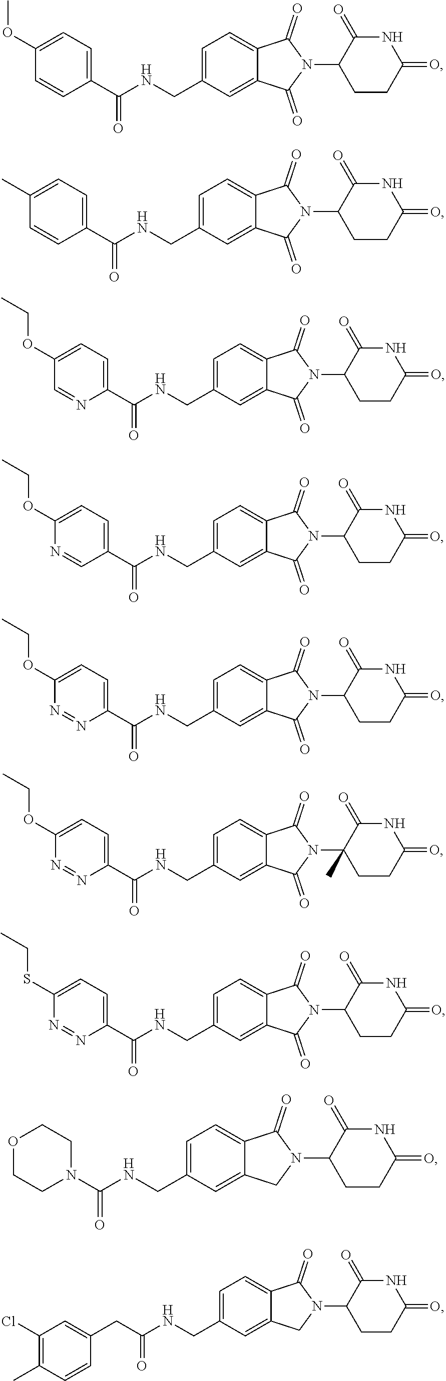

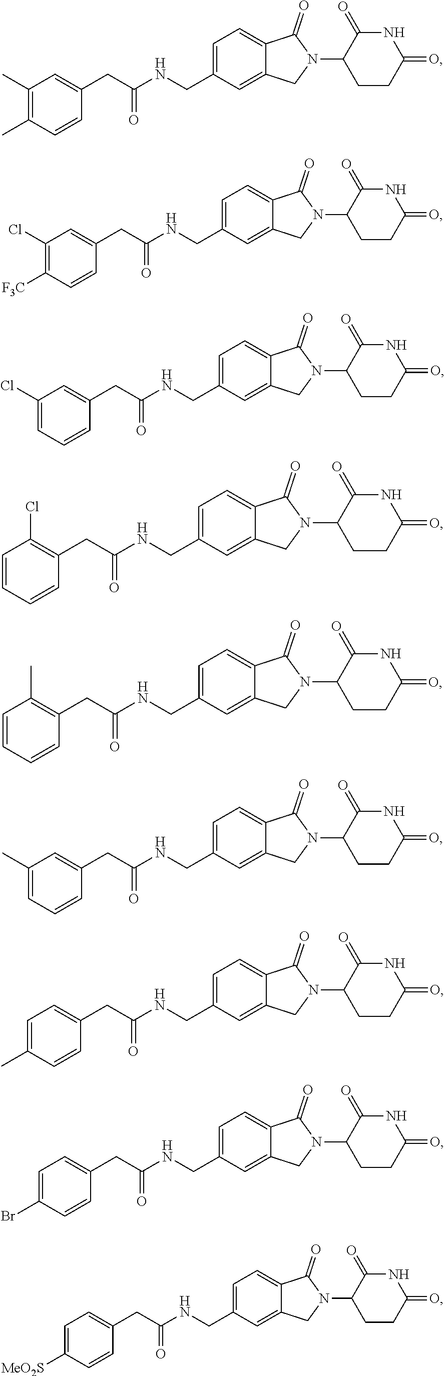

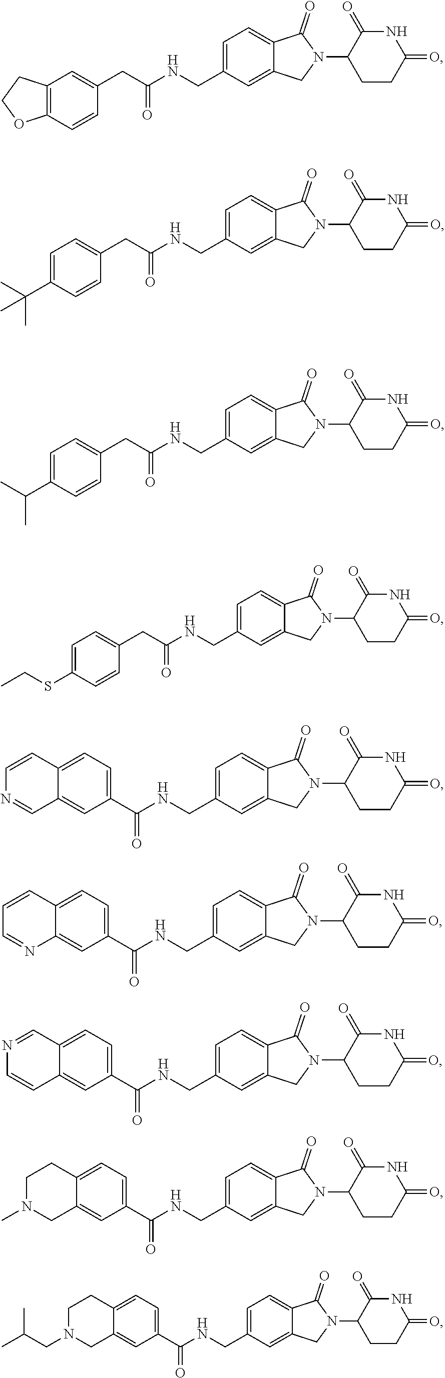

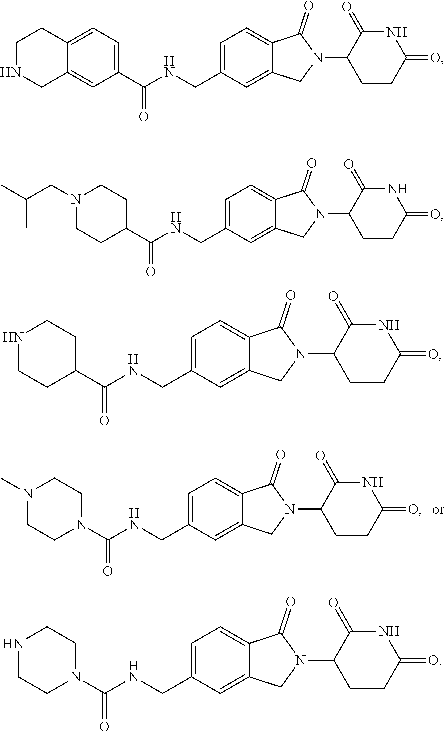 [SMILES: O=C1CCC(N2Cc3cc(CNC(=O)c4ccc5c(c4)CNCC5)ccc3C2=O)C(=O)N1 ; CC(C)CN1CCC(C(=O)NCc2ccc3c(c2)CN(C2CCC(=O)NC2=O)C3=O)CC1 ; O=C1CCC(N2Cc3cc(CNC(=O)C4CCNCC4)ccc3C2=O)C(=O)N1 ; CN1CCN(C(=O)NCc2ccc3c(c2)CN(C2CCC(=O)NC2=O)C3=O)CC1 ; O=C1CCC(N2Cc3cc(CNC(=O)N4CCNCC4)ccc3C2=O)C(=O)N1]

In certain aspects, the structure of the immunomodulatory imide compound of Formula (a) is

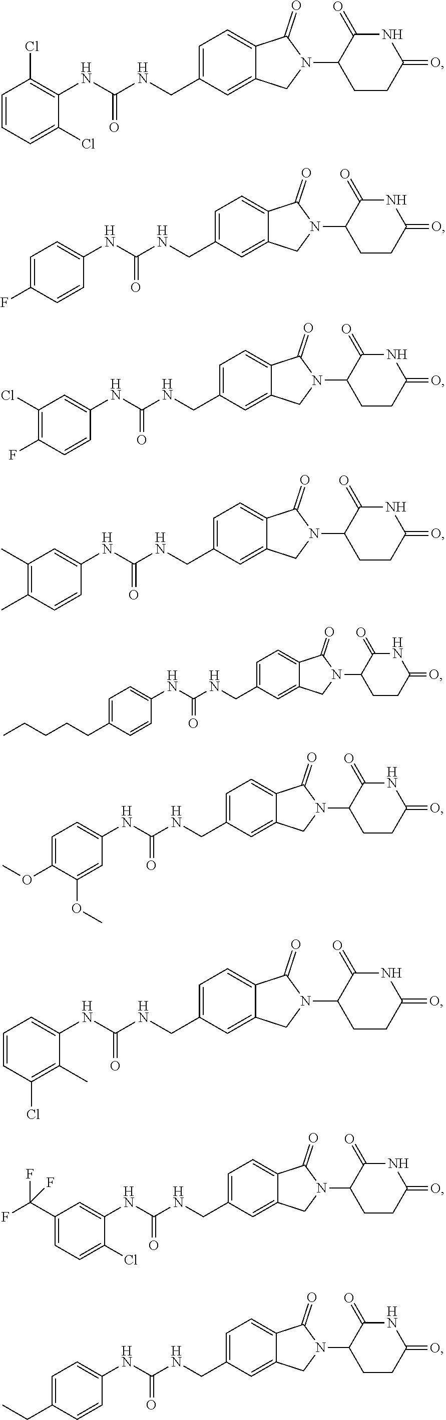

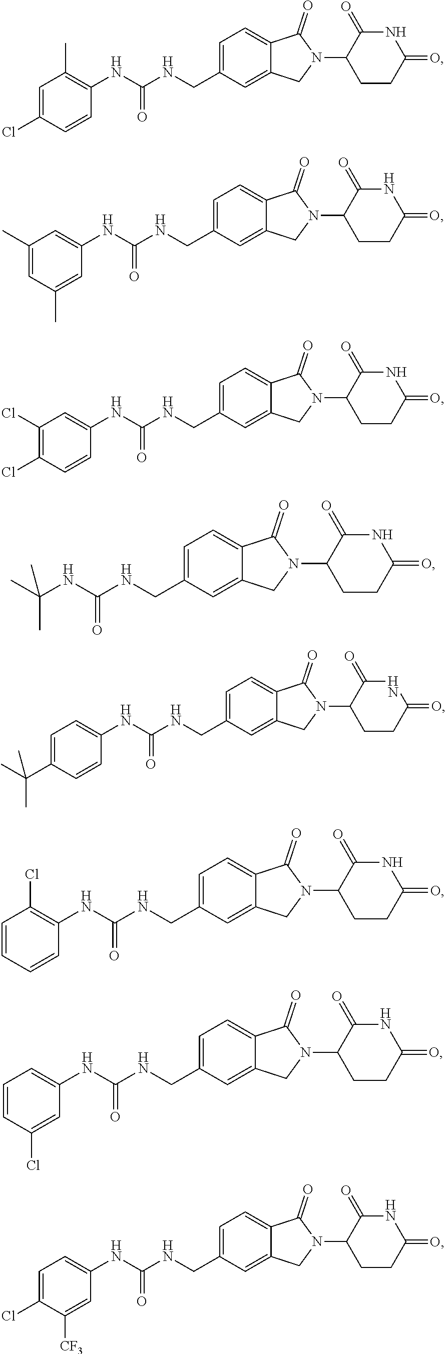

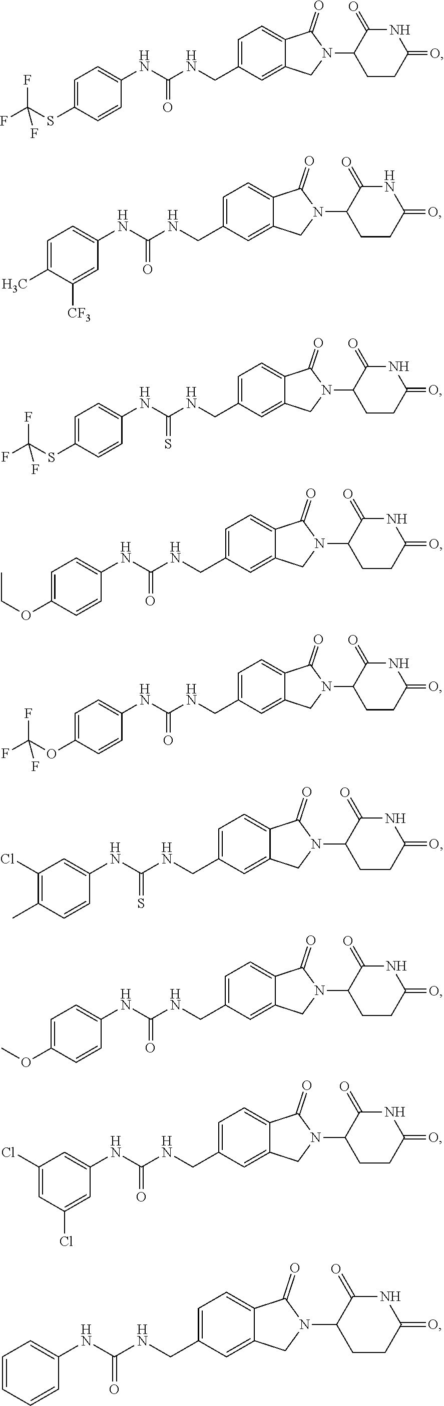

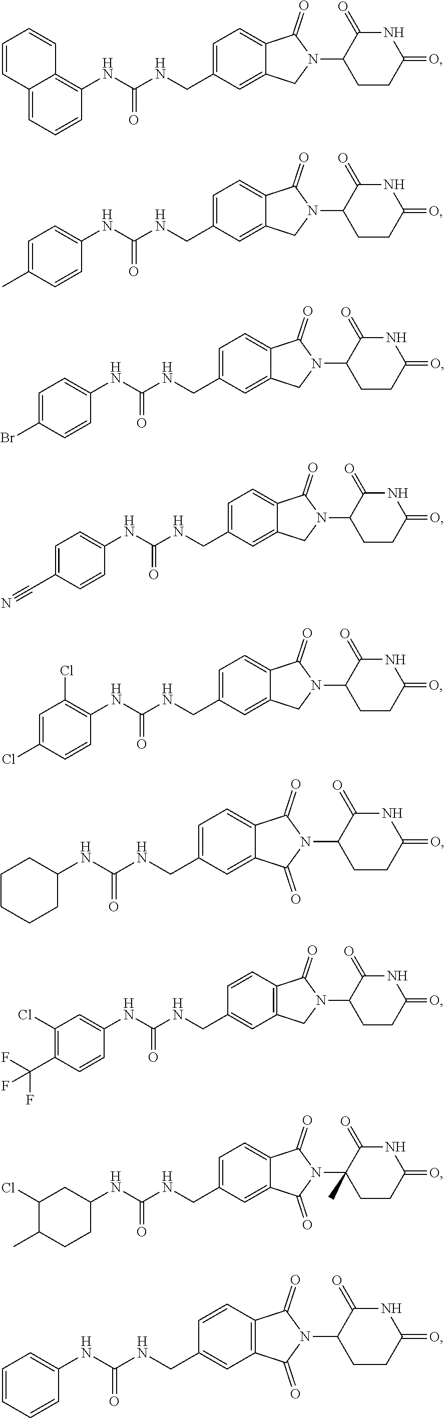

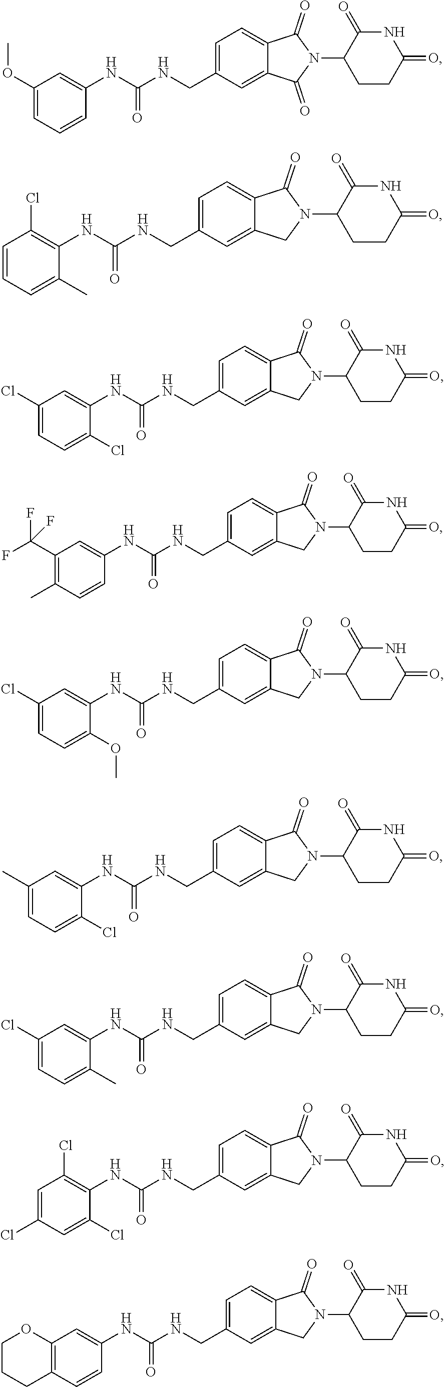

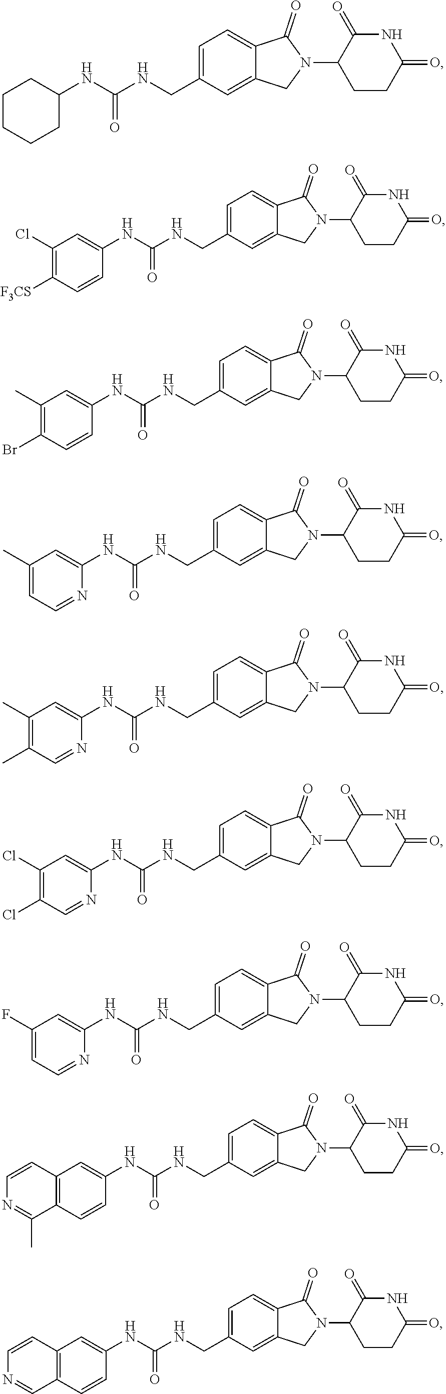

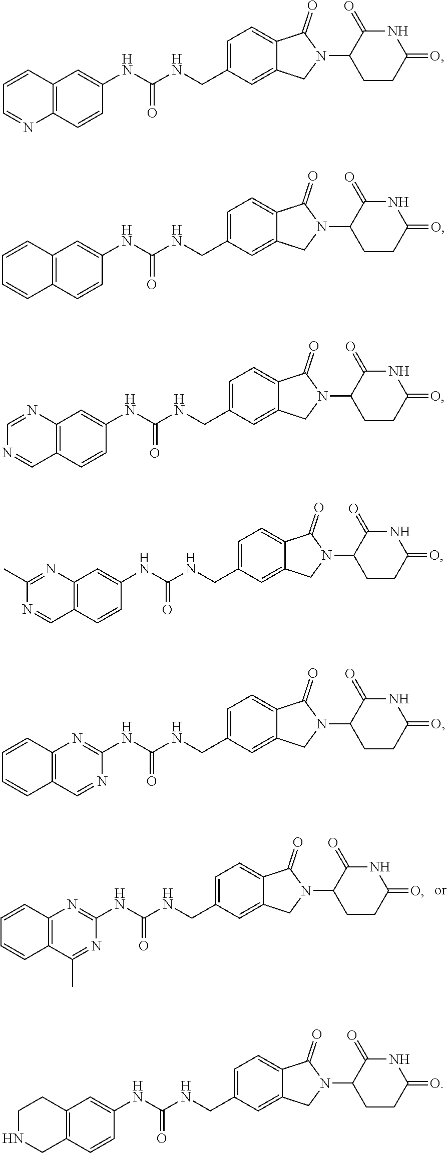

In certain aspects, the immunomodulatory imide compound is Formula (b):

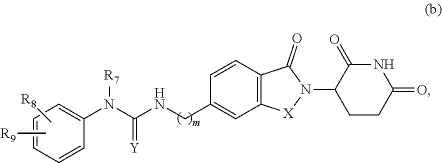

or a pharmaceutically acceptable salt, solvate, or stereoisomer thereof, wherein:
   X is C(═O) or CH₂;
   Y is O, cyanamido (N—C═N), or amido (NH);
   m is an integer of 0, 1, 2, or 3;
   R₇ is hydrogen or C₁₋₆ alkyl;
   R₈ is hydrogen, —NO₂, C₁₋₁₀ alkyl, C₀₋₆ alkyl-(5 to 10 membered heteroaryl), C₀₋₆ alkyl-(5 to 6 membered heterocyclyl), C₀₋₆ alkyl-OH, C₀₋₄ alkyl-NH₂, —NHCO—C₁₋₆ alkyl, —OR₂₁, or —(CH₂—Z)₀₋₂-(5 to 10 membered heteroaryl), where each heteroaryl and heterocyclyl is optionally substituted with one or more C₁₋₆ alkyl;
   R₉ is hydrogen, halogen, —NO₂, C₀₋₆ alkyl-(5 to 10 membered heteroaryl), C₀₋₆ alkyl-(5 to 6 membered heterocyclyl), C₀₋₆ alkyl-OH, C₀₋₄ alkyl-NH₂, —NHCO—C₁₋₆ alkyl, —OR₂₁, or —(CH₂—Z)₀₋₂-(5 to 10 membered heteroaryl), where each heteroaryl and heterocyclyl is optionally substituted with one or more C₁₋₆ alkyl;
   R₂₁ is C₆₋₁₀ aryl, 5 to 10 membered heteroaryl, 5 to 6 membered heterocyclyl, or —CO(CH₂)₀₋₂R₂₂, wherein the aryl, heteroaryl, and heterocyclyl are each optionally substituted with one or more C₁₋₆ alkyl;
   R₂₂ is —NH₂ or 5 to 6 membered heterocyclyl; and
   Z is CH₂, NH, or O.

In certain aspects, the structure of the immunomodulatory imide compound of Formula (b) is that when R₇ is hydrogen, then R₈ is not hydrogen or C_{1_6} alkyl; when Y is O, then R₉ is not halogen; and when Y is O and R₉ is halogen, then R₈ is C_{0_6} alkyl-(5-6 membered heterocyclyl).

In certain aspects, the immunomodulatory imide compound is Formula (c):

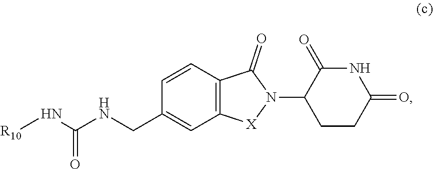

or a pharmaceutically acceptable salt, solvate, or stereoisomer thereof, wherein:
   X is C(═O) or CH₂;
   m is an integer of 0, 1, 2, or 3;
   R₁₀ is C₃₋₁₀ cycloalkyl, 5 to 10 membered heterocyclyl, 5 to 10 membered heteroaryl,
   or C₀₋₄ alkyl-NR₄₁R₄₂; wherein the cycloalkyl, heterocyclyl, and heteroaryl are each optionally
   substituted with one or more halogen, C₁₋₆ alkyl, —CO—NR₄₃R₄₄, —COOR₄₅, or C₀₋₄ alkyl-C₆₋₁₀ aryl,
   wherein the aryl itself may be optionally substituted with one or more halogen; and
   R₄₁, R₄₂, R₄₃, R₄₄, and R₄₅ are each independently hydrogen or C₁₋₆ alkyl.

In certain aspects, the immunomodulatory imide compound is Formula (d)

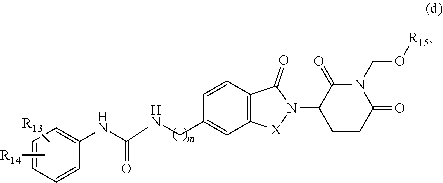

or a pharmaceutically acceptable salt, solvate, or stereoisomer thereof, wherein:
   X is C(═O) or CH₂;
   m is an integer of 0, 1, 2, or 3;
   R₁₃ and R₁₄ are each independently: hydrogen, halo, C₁₋₆ alkyl, oxo, —NO₂, —Z—C₀₋₆ alkyl-(5 to 10 membered heteroaryl), C₀₋₆ alkyl-(5 to 6 membered heterocyclyl), C₀₋₆ alkyl-OH, C₀₋₄ alkyl-NH₂, —NHCO—C₁₋₆ alkyl, —OR₂₁, or —(CH₂—Z)₀₋₂-(5 to 10 membered heteroaryl),
   wherein Z is S or SO₂;
   wherein R₂₁ is defined above;
   wherein each heteroaryl and heterocyclyl above is optionally substituted with one or more C₁₋₆ alkyl; and
   wherein the alkyl or alkoxy above may be optionally substituted with one or more: halogen; cyano; nitro; amino: C₁₋₆ alkylidenedioxy; C₁₋₆ alkoxy, itself optionally substituted with one or more halogens; or C₁₋₆ alkylthio, itself optionally substituted with one or more halogens:
   R₁₅ is COR₇₁ or PO(OR₇₂)(OR₇₃);
   R₇₁ is C₁₋₁₀ alkyl, C₆₋₁₀ aryl, or 5 to 6 membered heterocyclyl; wherein the alkyl, aryl, heterocyclyl may be optionally substituted with one or more amino, C₁₋₆ alkylamino, di(C₁₋₆ alkyl)amino, or —COOR₇₄; and
   R₇₂, R₇₃, and R₇₄ are each independently hydrogen or C₁₋₁₀ alkyl.

In certain aspects, the immunomodulatory imide compound is Formula (e)

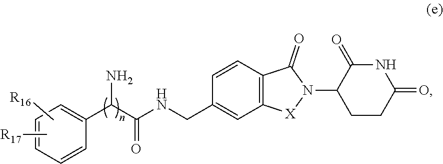

or a pharmaceutically acceptable salt, solvate, or stereoisomer thereof, wherein:
   X is C(═O) or CH:
   n is an integer of 0 or 1;
   R is hydrogen or halo; and
   R is hydrogen, amino, or 5 to 10 membered heteroaryl or heterocyclyl.

In certain aspects, the structure of the immunomodulatory imide compound of Formula (b) is that when m is 0, R₁₇ is not hydrogen.

In certain aspects, the immunomodulatory imide compound is Formula (f)

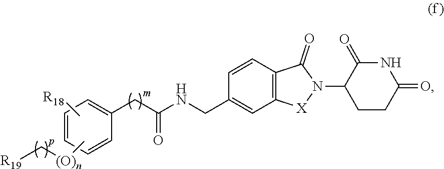

or a pharmaceutically acceptable salt, solvate, or stereoisomer thereof, wherein:
   X is CH or C═O;
   m and n are each independently 0 or 1;
   p is 0, 1, 2, or 3:
   R₁₉ is 5 to 6 membered heterocyclyl, optionally substituted with C₁₋₆ alkyl; and
   R₁₈ is hydrogen or halogen.

In certain aspects, the immunomodulatory imide compound is Formula (g)

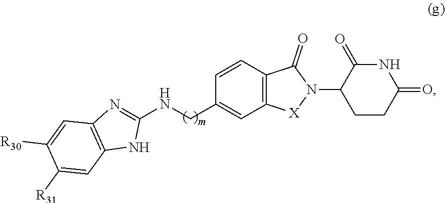

or a pharmaceutically acceptable salt, Solvate, prodrug, or stereoisomer thereof, wherein:
X is CH or C═O;
m is an integer of 0, 1, 2, or 3:
R₃₀ and R₃₁ are each independently hydrogen, halo, C₁₋₆ alkyl, or Caryloxy, wherein the alkyl and aryl are each optionally substituted with one or more halo.

### V. Compositions and Methods of Using

The conjugates and/or compounds described herein can be in the form of pharmaceutically or pharmaceutically acceptable salts. In some aspects, such salts are derived from inorganic or organic acids or bases.

Examples of suitable acid addition salts include acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Examples of suitable base addition salts include ammonium salts; alkali metal salts, such as sodium and potassium salts; alkaline earth metal salts, such as calcium and magnesium salts; salts with organic bases, such as dicyclohexylamine salts, N-methyl-D-glucamine; and salts with amino acids such as arginine, lysine, and the like.

For example, Berge lists the following FDA-approved commercially marketed salts: anions acetate, besylate (benzenesulfonate), benzoate, bicarbonate, bitartrate, bromide, calcium edetate (ethylenediaminetetraacetate), camsylate (camphorsulfonate), carbonate, chloride, citrate, dihydrochloride, edetate (ethylenediaminetetraacetate), edisylate (1,2-ethanedisulfonate), estolate (lauryl sulfate), esylate (ethanesulfonate), fumarate, gluceptate (glucoheptonate), gluconate, glutamate, glycollylarsanilate (glycollamidophenylarsonate), hexylresorcinate, hydrabamine (N,N′-di(dehydroabietyl)ethylenediamine), hydrobromide, hydrochloride, hydroxynaphthoate, iodide, isethionate (2-hydroxyethanesulfonate), lactate, lactobionate, malate, maleate, mandelate, mesylate (methanesulfonate), methylbromide, methylnitrate, methylsulfate, mucate, napsylate (2-naphthalenesulfonate), nitrate, pamoate (embonate), pantothenate, phosphate/diphosphate, polygalacturonate, salicylate, stearate, subacetate, succinate, sulfate, tannate, tartrate, teoclate (8-chlorotheophyllinate) and triethiodide; organic cations benzathine (N,N′-dibenzylethylenediamine), chloroprocaine, choline, diethanolamine, ethylenediamine, meglumine (N-methylglucamine) and procaine; and metallic cations aluminum, calcium, lithium, magnesium, potassium, sodium and zinc.

Berge additionally lists the following non-FDA-approved commercially marketed (outside the United States) salts: anions adipate, alginate, aminosalicylate, anhydromethylenecitrate, arecoline, aspartate, bisulfate, butylbromide, camphorate, digluconate, dihydrobromide, disuccinate, glycerophosphate, hemisulfate, hydrofluoride, hydroiodide, methylenebis(salicylate), napadisylate (1,5-naphthalenedisulfonate), oxalate, pectinate, persulfate, phenylethylbarbiturate, picrate, propionate, thiocyanate, tosylate and undecanoate; organic cations benethamine (N-benzylphenethylamine), clemizole (1-p-chlorobenzyl-2-pyrrolildine-1′-ylmethylbenzimidazole), diethylamine, piperazine and tromethamine (tris(hydroxymethyl)aminomethane); and metallic cations barium and bismuth.

Pharmaceutical compositions comprising the conjugates described herein may also comprise suitable carriers, excipients, and auxiliaries that may differ depending on the mode of administration.

In some aspects, the pharmaceutical compositions can be formulated as a suitable parenteral dosage form. Said formulations can be prepared by various methods known in the art. The pharmaceutical compositions can be administered directly into the bloodstream, into muscle, or directly into an organ. Suitable means for parenteral administration include intravenous, intraarterial, intraperitoneal, intrathecal, intraventricular, intraurethral, intrasternal, intracranial, intramuscular, and subcutaneous. Suitable devices for parenteral administration include needle injectors, needle-free injectors, and infusion techniques.

Parenteral compositions are typically aqueous solutions which may contain excipients such as salts, carbohydrates and buffering agents. However, the composition may also be formulated a sterile non-aqueous solution or as a dried form to be used in conjunction with a suitable vehicle such as sterile pyrogen-free water.

The preparation of parenteral compositions under sterile conditions, for example, by lyophilization, can be readily accomplished using standard techniques known well to those of skill in the art.

Compositions for parenteral administration can be formulated to be immediate and/or modified release. Modified release formulations include delayed-, sustained-, pulsed-, controlled-, targeted, and programmed release. Thus, the compositions can be formulated as a solid, semi-solid, or thixotropic liquid for administration as an implanted depot providing modified release of the active agent.

The parenteral formulations can be admixed with other suitable pharmaceutically acceptable excipients used in parenteral dosage forms such as, but not limited to, preservatives.

In another aspect, the pharmaceutical compositions can be formulated as suitable oral dosage forms such as tablets, capsules, powders, pellets, suspensions, solutions, emulsions, and the like. Other suitable carriers can be present such as disintegrants, diluents, chelating agents, binders, glidants, lubricants, fillers, bulking agents, anti-adherents, and the like.

Oral dosage formulations may also contain other suitable pharmaceutical excipients such as sweeteners, vehicle/wetting agents, coloring agents, flavoring agents, preservatives, viscosity enhancing/thickening agents, and the like.

The conjugates described herein can be used to treat various cancers. Certain conjugates of the present disclosure can be superior in terms of efficacy expression, pharmacokinetics (e.g., absorption, distribution, metabolism, excretion), solubility (e.g., water solubility), interaction with other medicaments (e.g., drug-metabolizing enzyme inhibitory action), safety (e.g., acute toxicity, chronic toxicity, genetic toxicity, reproductive toxicity, cardiotoxicity, carcinogenicity, central toxicity) and/or stability (e.g., chemical stability, stability to an enzyme), and can be useful as a medicament.

The conjugates of the present disclosure can be used as medicaments such as an agents for the prophylaxis or treatment of diseases, for example, cancers—e.g., colorectal cancers (e.g., colorectal cancer, rectal cancer, anus cancer, familial colorectal cancer, hereditary nonpolyposis colorectal cancer, gastrointestinal stromal tumor), lung cancers (e.g., non-small-cell lung cancer, small-cell lung cancer, malignant mesothelioma), mesothelioma, pancreatic cancers (e.g., pancreatic ductal carcinoma, pancreatic endocrine tumor), pharynx cancer, larynx cancer, esophageal cancer, stomach/gastric cancers (e.g., papillary adenocarcinoma, mucinous adenocarcinoma, adenosquamous carcinoma), duodenal cancer, small intestinal cancer, breast cancers (e.g., invasive ductal carcinoma, non-invasive ductal carcinoma, inflammatory breast cancer), ovarian cancers (e.g., ovarian epithelial cancer, extragonadal germ cell tumor, ovarian germ cell tumor, ovarian low-malignant potential tumor), testis tumor, prostate cancers (e.g., hormone-dependent prostate cancer, non-hormone dependent prostate cancer, castration-resistant prostate cancer), liver cancers (e.g., hepatocellular cancer, primary liver cancer, extrahepatic bile duct cancer), thyroid cancers (e.g., medullary thyroid carcinoma), renal cancers (e.g., renal cell cancers (e.g., clear cell renal cell cancer), transitional cell cancer of renal pelvis and ureter), uterine cancers (e.g., cervical cancer, uterine body cancer, uterus sarcoma), gestational choriocarcinoma, brain tumors (e.g., medulloblastoma, glioma, pineal astrocytic tumors, pilocytic astrocytoma, diffuse astrocytoma, anaplastic astrocytoma, pituitary adenoma), retinoblastoma, skin cancers (e.g., basalioma, malignant melanoma), sarcomas (e.g., rhabdomyosarcoma, leiomyosarcoma, soft tissue sarcoma, spindle cell sarcoma), malignant bone tumor, bladder cancer, hematological/blood cancers (e.g., multiple myeloma, leukemias (e.g., acute myelogenous leukemia), malignant lymphoma, Hodgkin's disease, chronic myeloproliferative disease), cancer of unknown primary; a cancer growth inhibitor; a cancer metastasis inhibitor; an apoptosis promoter; an agent for the treatment of precancerous lesions (e.g., myelodysplastic syndromes); and the like.

In certain aspects, conjugates of the present disclosure can be used as a medicament for breast cancer, gastric cancer, ovarian cancer, uterine cancer, lung cancer, pancreatic cancer, liver cancer, lymphoma, or hematological cancers.

Furthermore, conjugates of the present disclosure or can be used concurrently with a non-drug therapy. To be precise, the conjugates can be combined with a non-drug therapy such as (1) surgery, (2) hypertensive chemotherapy using angiotensin II etc., (3) gene therapy, (4) thermotherapy, (5) cryotherapy, (6) laser cauterization and (7) radiotherapy.

For example, by using a conjugate of the present disclosure before or after the above-mentioned surgery and the like, effects such as prevention of emergence of resistance, prolongation of Disease-Free Survival, suppression of cancer metastasis or recurrence, prolongation of life and the like may be afforded.

In addition, it is possible to combine a treatment with conjugates of the present disclosure with a supportive therapy: (i) administration of antibiotic (e.g., P-lactam type such as pansporin and the like, macrolide type such as clarithromycin and the like) for the complication with various infectious diseases, (ii) administration of high-calorie transfusion, amino acid preparation or general vitamin preparation for the improvement of malnutrition, (iii) administration of morphine for pain mitigation, (iv) administration of a pharmaceutical agent for ameliorating side effects such as nausea, vomiting, anorexia, diarrhea, leucopenia, thrombocytopenia, decreased hemoglobin concentration, hair loss, hepatopathy, renopathy, DIC, fever and the like and (v) administration of a pharmaceutical agent for suppressing multiple drug resistance of cancer and the like.

In some aspects, the conjugate of the disclosure can be used in combination with a standard of care therapy, e.g., one or more therapeutic agents (e.g., anti-cancer agents and/or immunomodulating agents). Accordingly, in certain aspects, a method of treating a tumor disclosed herein comprises administering the conjugate of the disclosure in combination with one or more additional therapeutic agents. In some aspects, the conjugate of the disclosure can be used in combination with one or more anti-cancer agents, such that multiple elements of the immune pathway can be targeted. In some aspects, an anti-cancer agent comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof. A comprehensive and non-limiting list of combination treatment is disclosed in detail in the Combination Treatments section of this application.

In some aspects, the conjugate of the disclosure is administered to the subject prior to or after the administration of the additional therapeutic agent. In other aspects, the conjugate of the disclosure is administered to the subject concurrently with the additional therapeutic agent. In certain aspects, the conjugate of the disclosure and the additional therapeutic agent can be administered concurrently as a single composition in a pharmaceutically acceptable carrier. In other aspects, the conjugate of the disclosure and the additional therapeutic agent are administered concurrently as separate compositions.

In some aspects, a subject that can be treated with the conjugate of the present disclosure is a nonhuman animal such as a rat or a mouse. In some aspects, the subject that can be treated is a human.

### IV. Methods of Preparing Conjugates and Compositions

The present disclosure provides a method of preparing the conjugates, the process comprising reacting a binding moiety with a structure L′-V, wherein
V is an immunomodulatory imide compound, e.g., 5-substituted isoindoline compound;
L′ is a cleavable or non-cleavable linker precursor that conjugates to the binding moiety.
   As described herein, the linker precursor contain a heterobifunctional group that connects to the binding moiety.

In some aspects, L′ is a non-cleavable linker precursor. In some aspects, L′ is selected from the group consisting of

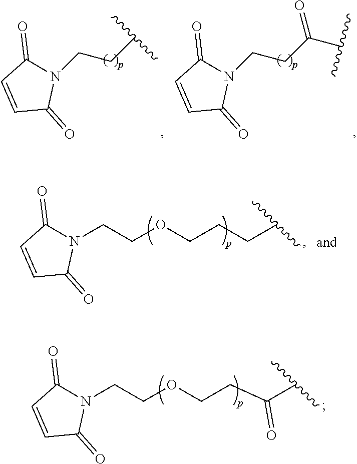

wherein:
   p is an integer from 1 to 10; and
   [symbol] is the point of attachment to X.

In some aspects, L′ is

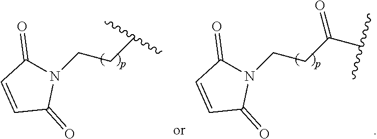

In some aspects, p is 5.

In certain aspects, L′ is a cleavable linker precursor.

In some aspects, the linker precursor is cleavable by a protease. In some aspects, the linker precursor is selected from the group consisting of

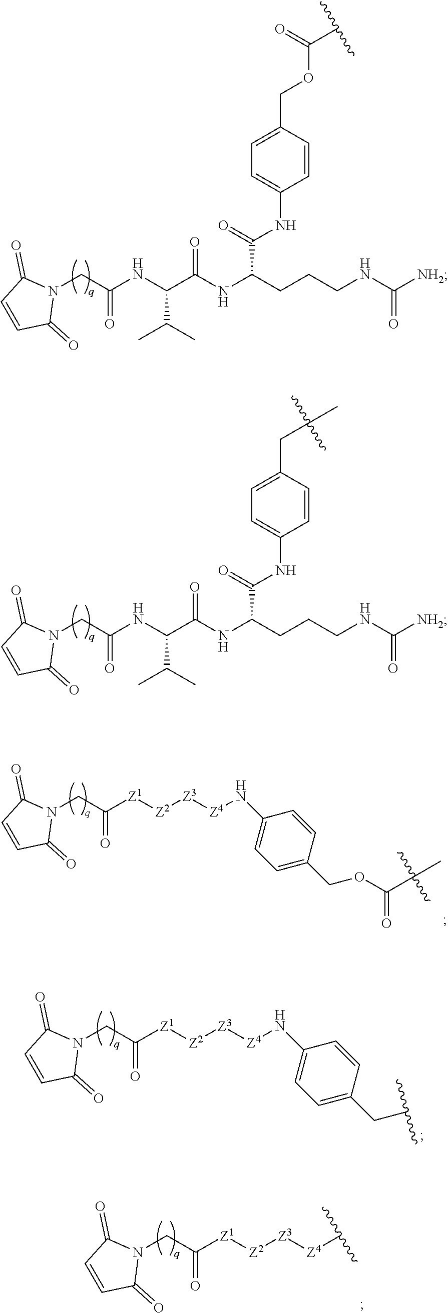

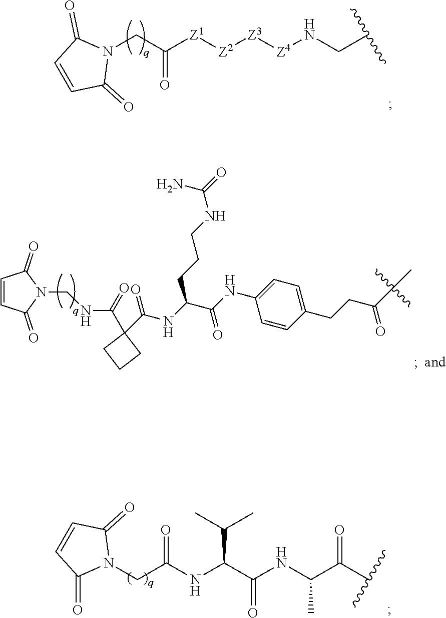

wherein:
   q is an integer from 2 to 10;
   Z¹, Z², Z³, and Z⁴ are each independently absent or a naturally-occurring amino acid residue in the L- or D-configuration, provided that at least two of Z¹, Z², Z³, and Z⁴ are amino acid residues; and
   [symbol] is the point of attachment to X.

In some aspects, Z¹, Z², Z³, and Z⁴ are independently absent selected from the group consisting of L-valine, D-valine, L-citrulline, D-citrulline, L-alanine, D-alanine, L-glutamine, D-glutaimine, L-glutamic acid, D-glutamic acid, L-aspartic acid, D-aspartic acid, L-asparagine, D-asparagine, L-phenylalanine, D-phenylalanine, L-lysine, D-lysine, and glycine, provided that at least two of Z¹, Z², Z³, and Z⁴ are amino acid residues.

In some aspects:
Z¹ is absent or glycine;
Z² is absent or selected from the group consisting of L-glutamine, D-glutamine, L-glutamic acid, D-glutamic acid, L-aspartic acid, D-aspartic acid, L-alanine, D-alanine, and glycine;
Z³ is selected from the group consisting of L-valine, D-valine, L-alanine, D-alanine, L-phenylalanine, D-phenylalanine, and glycine; and
Z⁴ is selected from the group consisting of L-alanine, D-alanine, L-citrulline, D-citrulline, L-asparagine, D-asparagine, L-lysine, D-lysine, L-phenylalamine, D-phenylalanine, and glycine.

In some aspects, L′ is

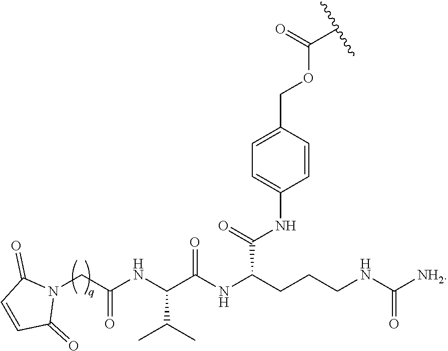

In some aspects, q is 5.

In some aspects, L′ is a bioreducible linker precursor. In some aspects, the bioreducible linker precursor is selected from the group consisting of

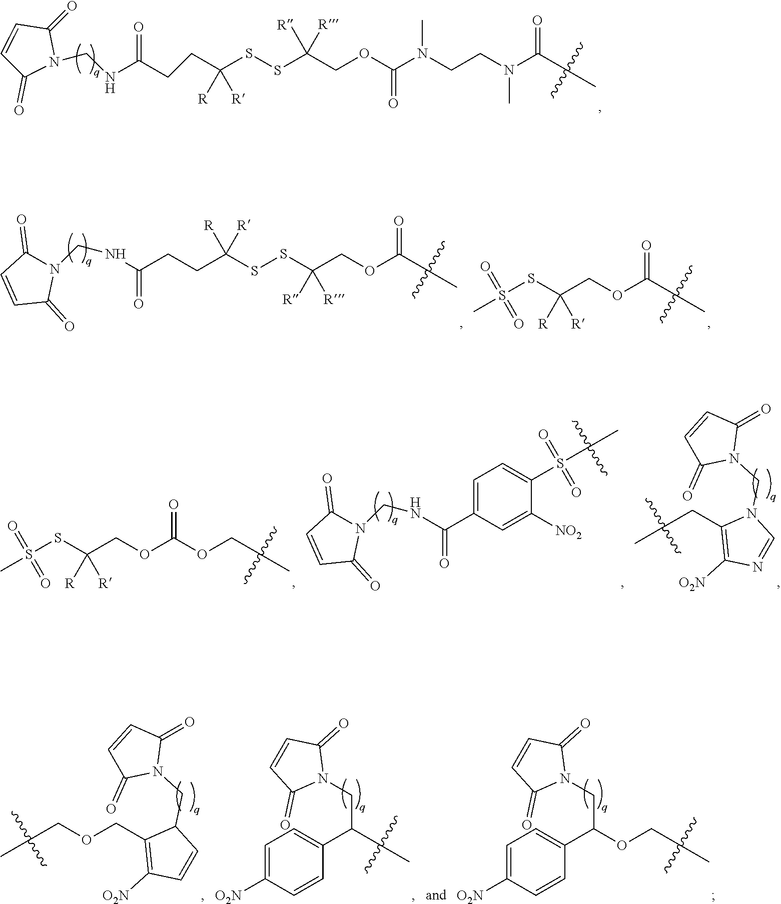

wherein:
   q is an integer from 2 to 10;
   R, R′, R″, and R′″ are each independently selected from hydrogen, C₁-C₆alkoxyC₁-C₆alkyl, (C₁-C₆)₂NC₁—C₆alkyl, and C₁-C₆alkyl, or, two geminal R groups, together with the carbon atom to which they are attached, can form a cyclobutyl or cyclopropyl ring; and
   [symbol] is the point of attachment to X.

In certain aspects, L′ is an acid cleavable linker precursor. In some aspects, L′ is selected from the group consisting of

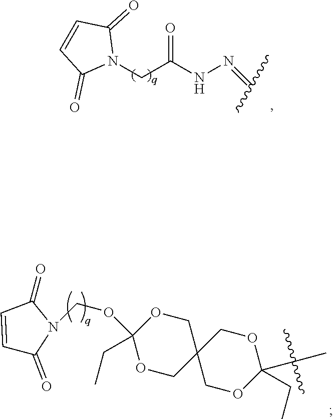

wherein:
   q is an integer from 2 to 10; and
   [symbol] is the point of attachment to X.

In certain aspects, L′ is a click-to-release linker precursor. In some aspects, L′ is selected from

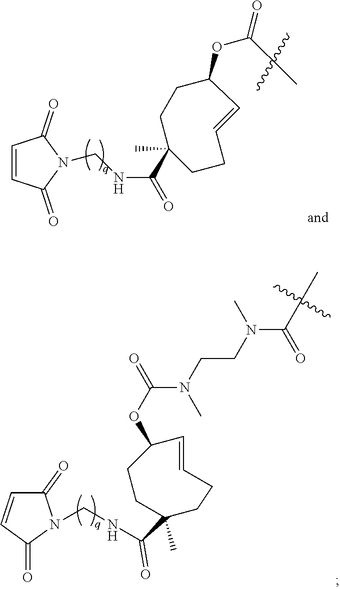

wherein:
   q is an integer from 2 to 10; and
   [symbol] is the point of attachment to X.

In certain aspects, L′ is a pyrophosphatase cleavable linker precursor. In some aspects, L′ is

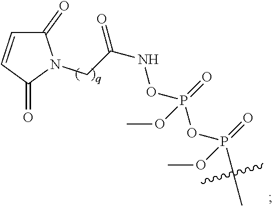

wherein:
   q is an integer from 2 to 10;
   [symbol] is the point of attachment to X.

In certain aspects, L′ is a beta-glucoronidase cleavable linker precursor. In some aspects, L′ is selected from

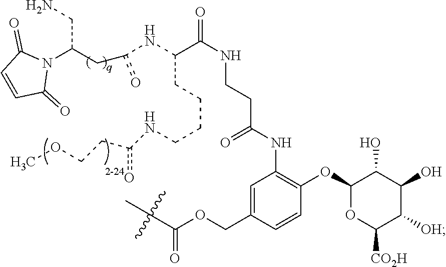

wherein:
   q is an integer from 2 to 10;
   ---- is absent or a bond; and
   [symbol] is the point of attachment to X.

In some aspects, the binding moiety is pre-treated before it is reacted with the structure (L′-V). In certain aspects, the structure (L′-V) is reacted with a binding moiety, which comprises an antibody or an antigen binding portion thereof. In aspects where the binding moiety is an antibody, the antibody can be pretreated to reduce interchain disulfides prior to reaction with the structure (L′-V).

In certain aspects, the immunomodulatory imide compound is Formula (a):

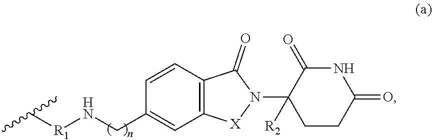

or a pharmaceutically acceptable salt, solvate, or stereoisomer thereof, wherein:
n is 0 or 1;
X is CH₂, C═O, or C═S;
[symbol] is the point of attachment to L;
R₁ is:
a) —(CH₂)ₘR₃ or —CO(CH₂)ₘR₃, wherein
   m is 0, 1, 2, or 3; and
   R₃ is 5-10 membered aryl or heteroaryl, optionally substituted with one or more halogen;
b) —C═YR₄, wherein
   Y is O or S; and
   R₄ is:
   (C₀-C₁₀)alkyl-(5 to 10 membered heteroaryl or heterocycle), said heteroaryl or heterocycle optionally substituted with one or more of (C₁-C₆)alkyl, halogen, oxo, (C₁-C₆)alkoxy, or —Z—(C₁-C₆)alkyl, wherein Z is S or SO₂, and wherein said (C₁-C₆)alkyl may be optionally substituted with one or more halogen;
   (C₀-C₁₀)alkyl-(5 to 10 membered aryl), said aryl optionally substituted with one or more of: halogen; (C₁-C₆)alkoxy, itself optionally substituted with one or more halogen; (C₁-C₆)alkyl, itself optionally substituted with one or more halogen; or —Z—(C₁-C₆)alkyl, wherein Z is S or SO₂, and wherein said (C₁-C₆)alkyl may be optionally substituted with one or more halogen; or (C₁-C₆)alkyl-CO—O—R₁₂, wherein R₁₂ is H or (C₁-C₆)alkyl; or
c) —C═ZNHR₆, wherein
   Z is O or S; and
   R₆ is:
   5 to 10 membered aryl or heteroaryl, optionally substituted with one or more of: halogen; cyano; (C₁-C₆)alkylenedioxy; (C₁-C₅)alkoxy, itself optionally substituted with one or more halogen; (C₁-C₆)alkyl, itself optionally substituted with one or more halogen; or (C₁-C₆)alkylthio, itself optionally substituted with one or more halogen; and
R₂ is H or (C₁-C₆)alkyl.

In certain aspects, the structure of the immunomodulatory imide compound of Formula (a) is that:
n is 0 or 1;
X is CH₂ or C═O;
R₁ is —(CH₂)ₘR₃, wherein
   m is 0, 1, 2, or 3; and
   R₃ is 5-10 membered aryl or heteroaryl, optionally substituted with one or more halogen.

In certain aspects, the structure of the immunomodulatory imide compound of Formula (a) is that:
n is 1;
X is CH₂ or C═O;
R₁ is:
   —C═OR₄, wherein
      R₄ is:
      (C₀-C₁₀)alkyl-(5 to 10 membered heteroaryl or heterocycle), said heteroaryl or
      heterocycle optionally substituted with one or more of (C₁-C₆)alkyl, halogen, oxo, (C₁-C₆)alkoxy, or —Z—(C₁-C₆)alkyl, wherein Z is S or SO₂, and wherein said (C₁-C₆)alkyl may be optionally substituted with one or more halogen;
      (C₀-C₁₀)alkyl-(5 to 10 membered aryl), said aryl optionally substituted with one or more of: halogen; (C₁-C₆)alkoxy, itself optionally substituted with one or
      more halogen; (C₁-C₆)alkyl, itself optionally substituted with one or more halogen; or —Z—(C₁-C₆)alkyl, wherein Z is S or SO₂, and wherein said (C₁-C₆)alkyl may be optionally substituted with one or more halogen; or (C₁-C₆)alkyl-CO—O—R₁₂, wherein R₁₂ is H or (C₁-C₆)alkyl.

In certain aspects, the structure of the immunomodulatory imide compound of Formula (a) is that:
n is 1;
X is CH₂ or C═O;
R₁ is:
   —C═ONHR₆, wherein
      R₆ is:
      5 to 10 membered aryl or heteroaryl, optionally substituted with one or more of: halogen; cyano; (C₁-C₆)alkylenedioxy; (C₁-C₅)alkoxy, itself optionally substituted with one or more halogen; (C₁-C₆)alkyl, itself optionally substituted with one or more halogen; or (C₁-C₆)alkylthio, itself optionally substituted with one or more halogen.

In certain aspects, the structure of the immunomodulatory imide compound of Formula (a) is that:
n is 1;
X is CH₂ or C═O;
R₁ is:
   —C═OR₄, wherein
   R₄ is:
   (C₀-C₁₀)alkyl-(5 to 10 membered heteroaryl or heterocycle), said heteroaryl or heterocycle optionally substituted with one or more of (C₁-C₆)alkyl, halogen, oxo, (C₁-C₆)alkoxy, or —Z—(C₁-C₆)alkyl, wherein Z is S or SO₂, and wherein said (C₁-C₆)alkyl may be optionally substituted with one or more halogen;
   (C₀-C₁₀)alkyl-(5 to 10 membered aryl), said aryl optionally substituted with one or more of: halogen; (C₁-C₆)alkoxy, itself optionally substituted with one or more halogen; (C₁-C₆)alkyl, itself optionally substituted with one or more halogen; or —Z—(C₁-C₆)alkyl, wherein Z is S or SO₂, and wherein said (C₁-C₆)alkyl may be optionally substituted with one or more halogen; or (C₁-C₆)alkyl-CO—O—R₁₂, wherein R₁₂ is H or (C₁-C₆)alkyl.

In certain aspects, the structure of the immunomodulatory imide compound of Formula (a) is

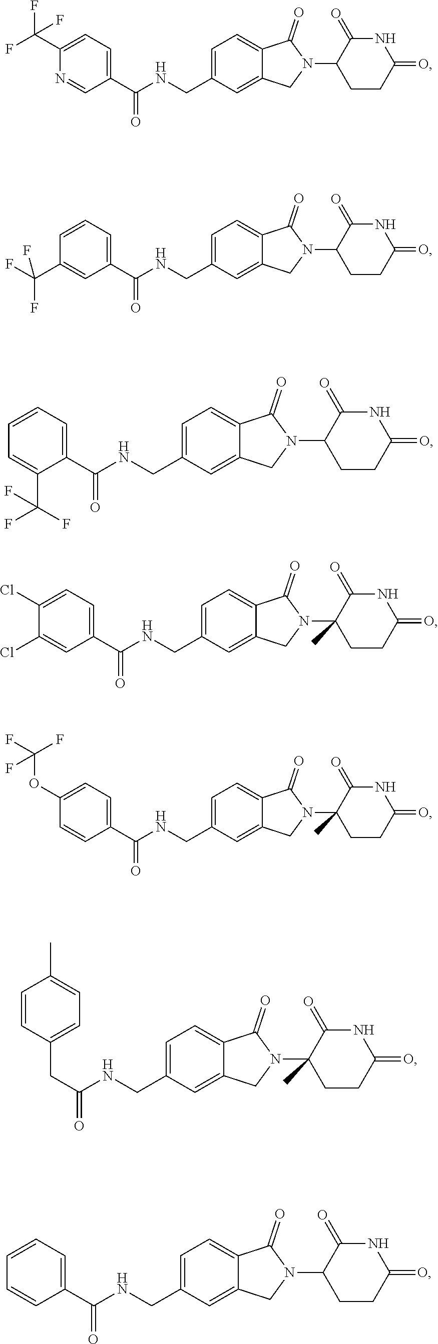

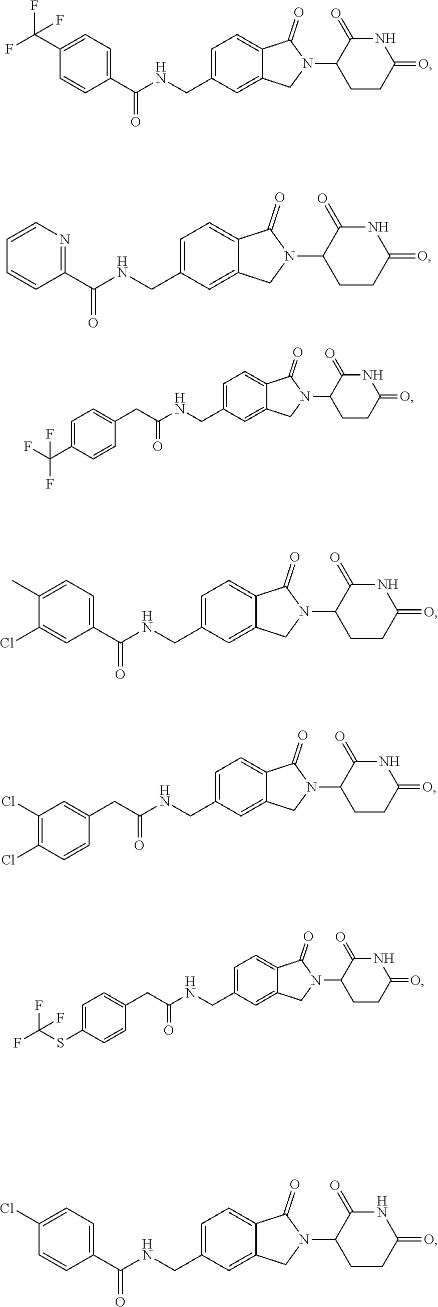

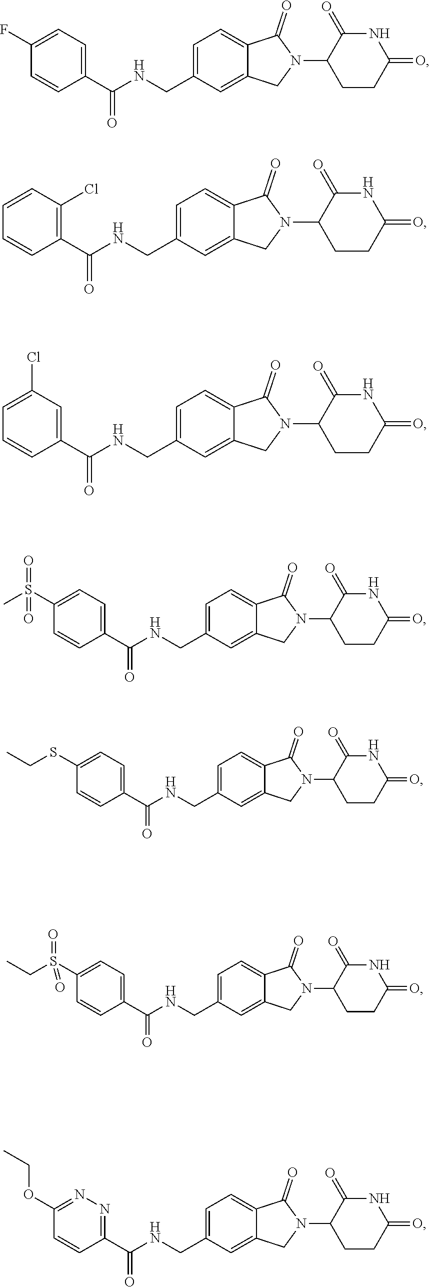

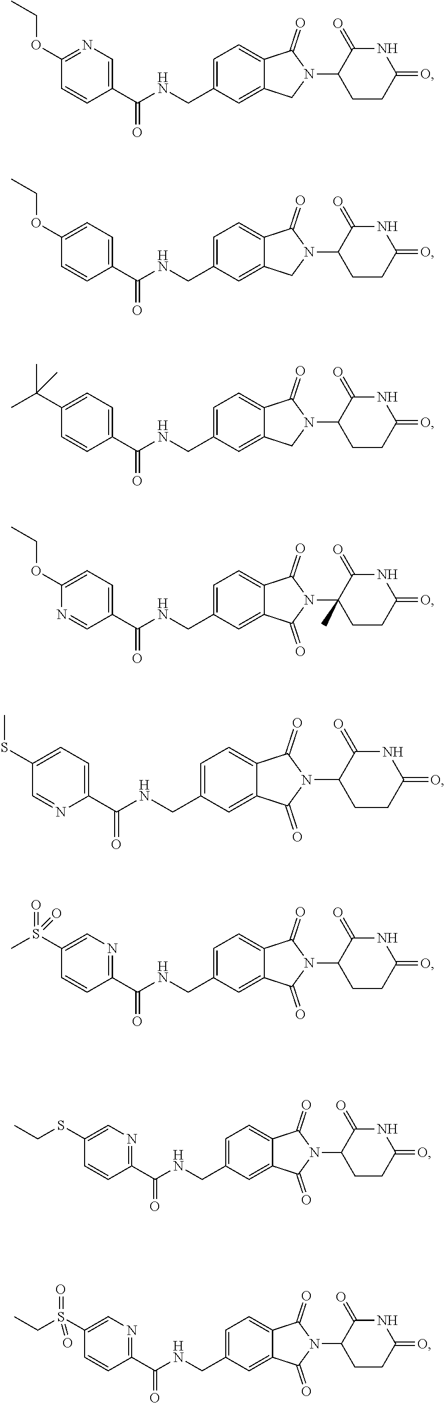

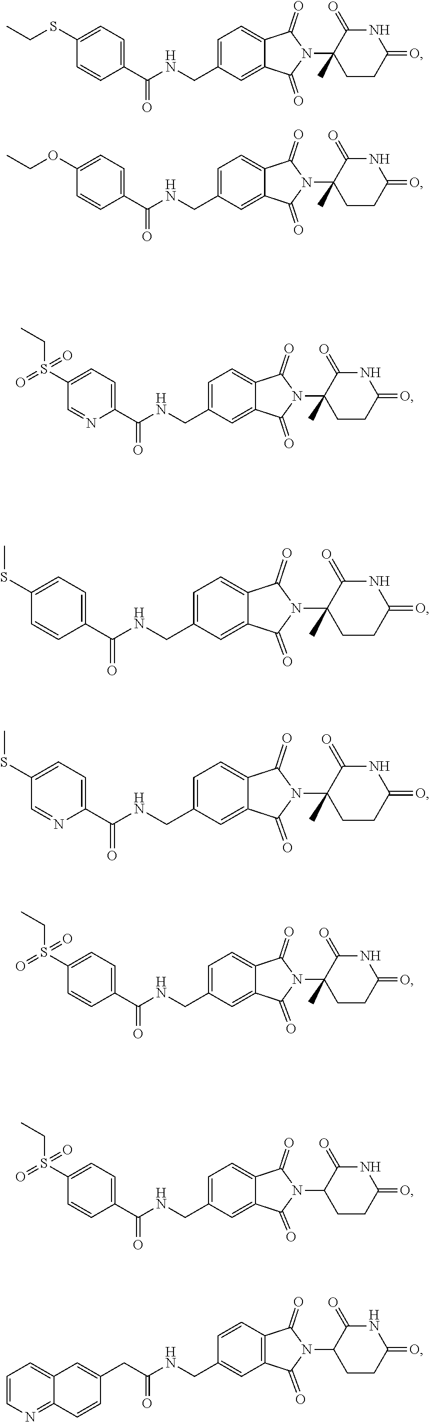

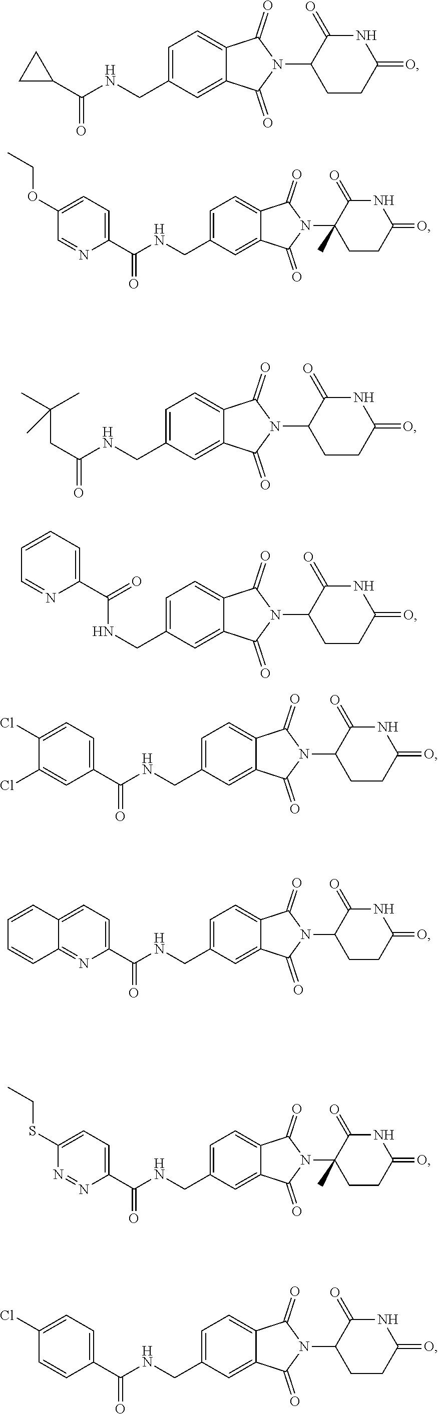

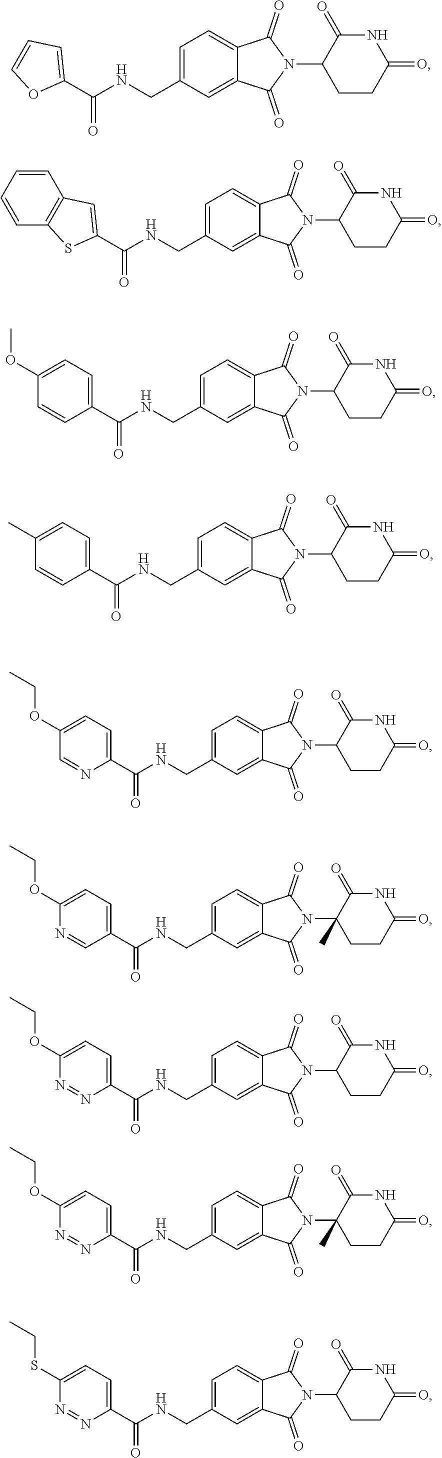

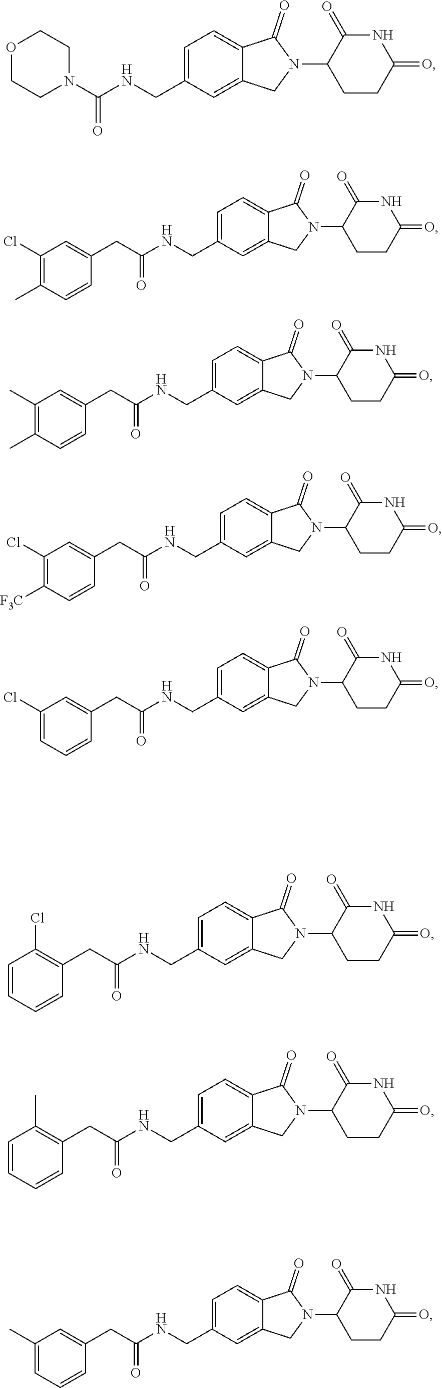

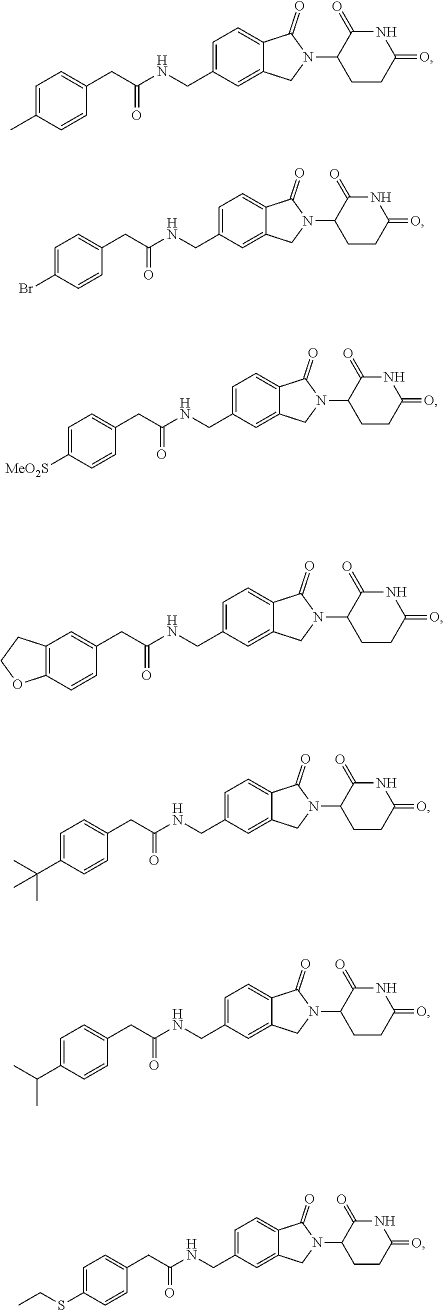

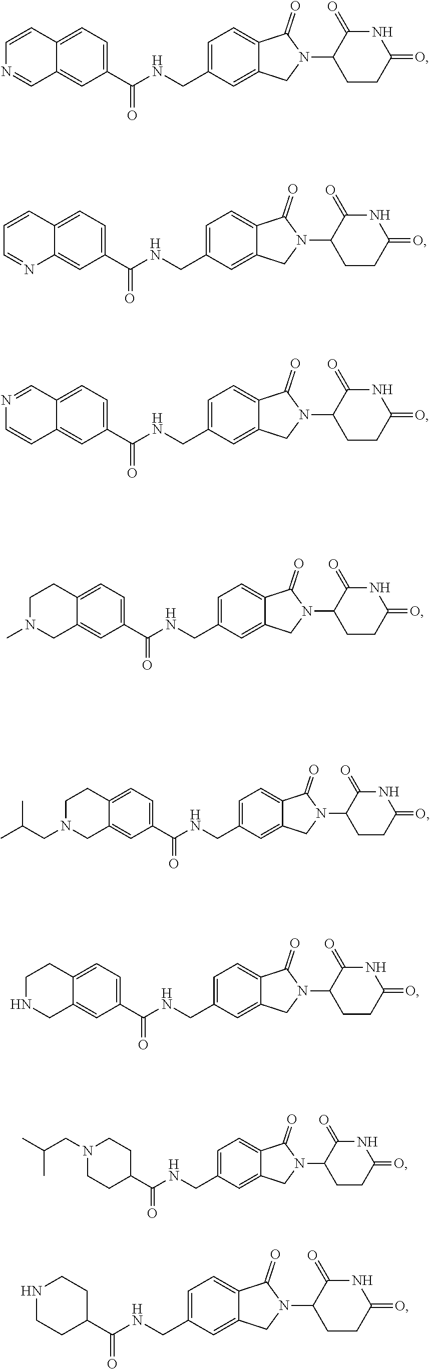

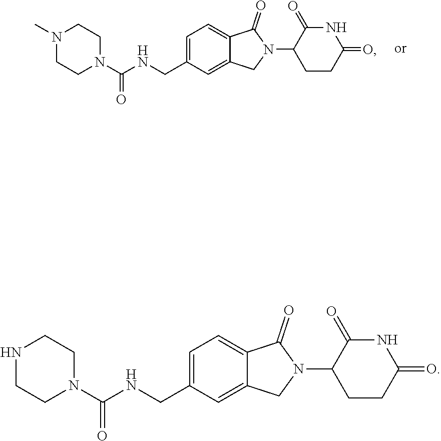 [SMILES: CN1CCN(C(=O)NCc2ccc3c(c2)CN(C2CCC(=O)NC2=O)C3=O)CC1 ; "or" ; O=C1CCC(N2Cc3cc(CNC(=O)N4CCNCC4)ccc3C2=O)C(=O)N1]

In certain aspects, the structure of the immunomodulatory imide compound of Formula (a) is

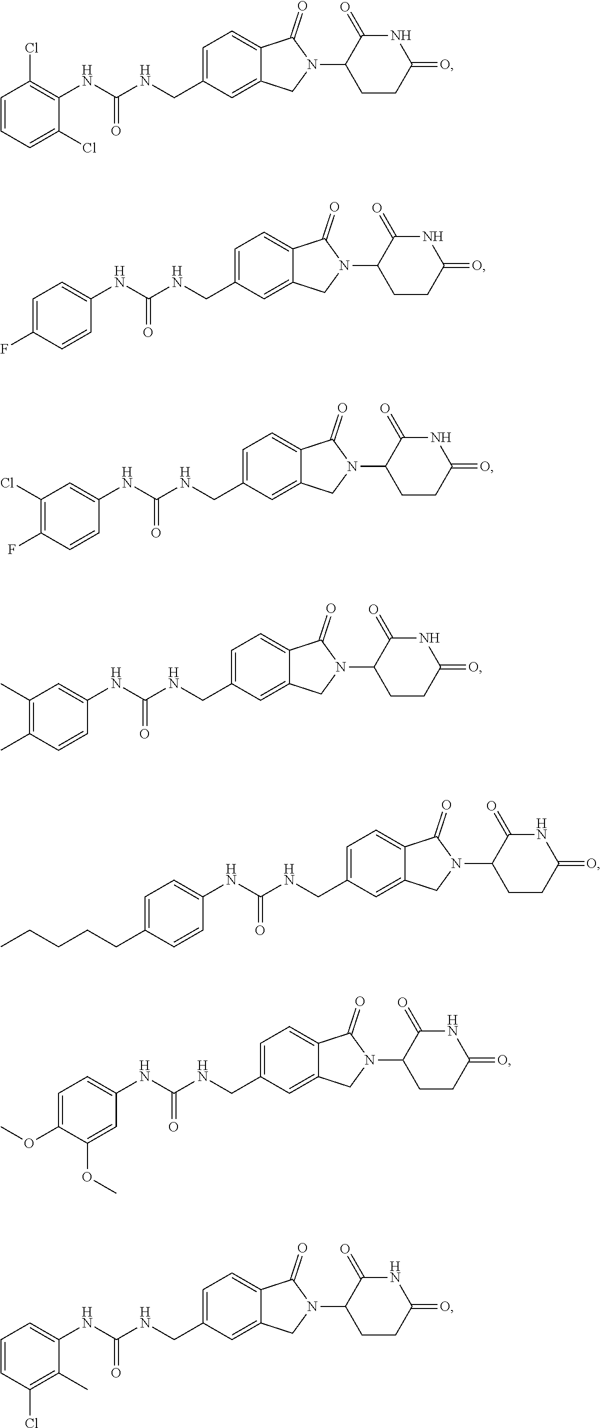

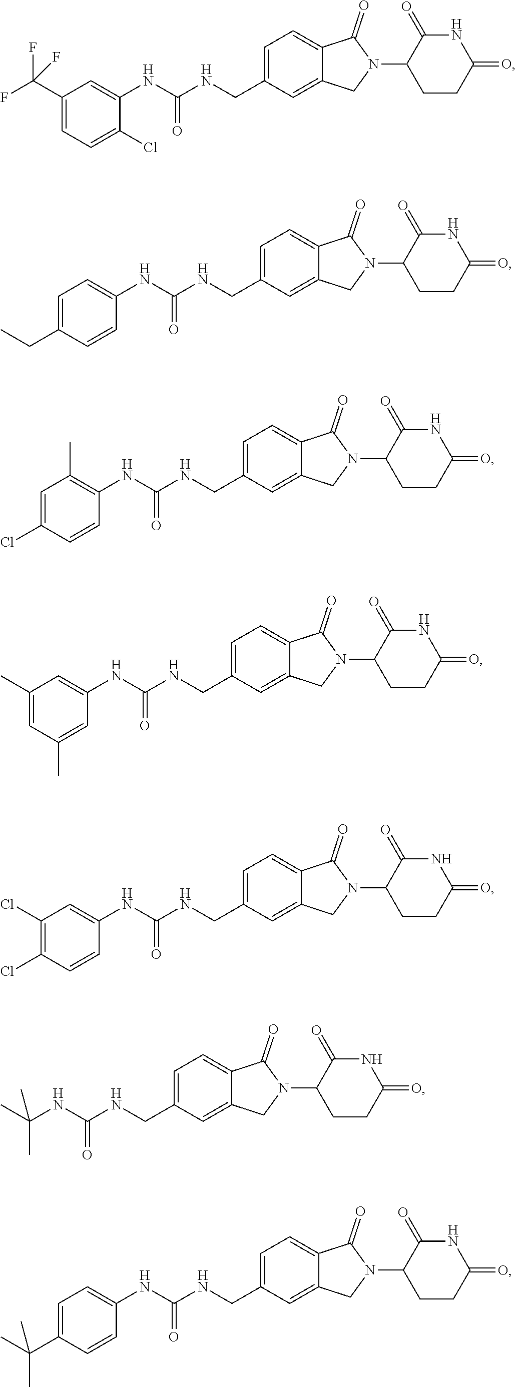

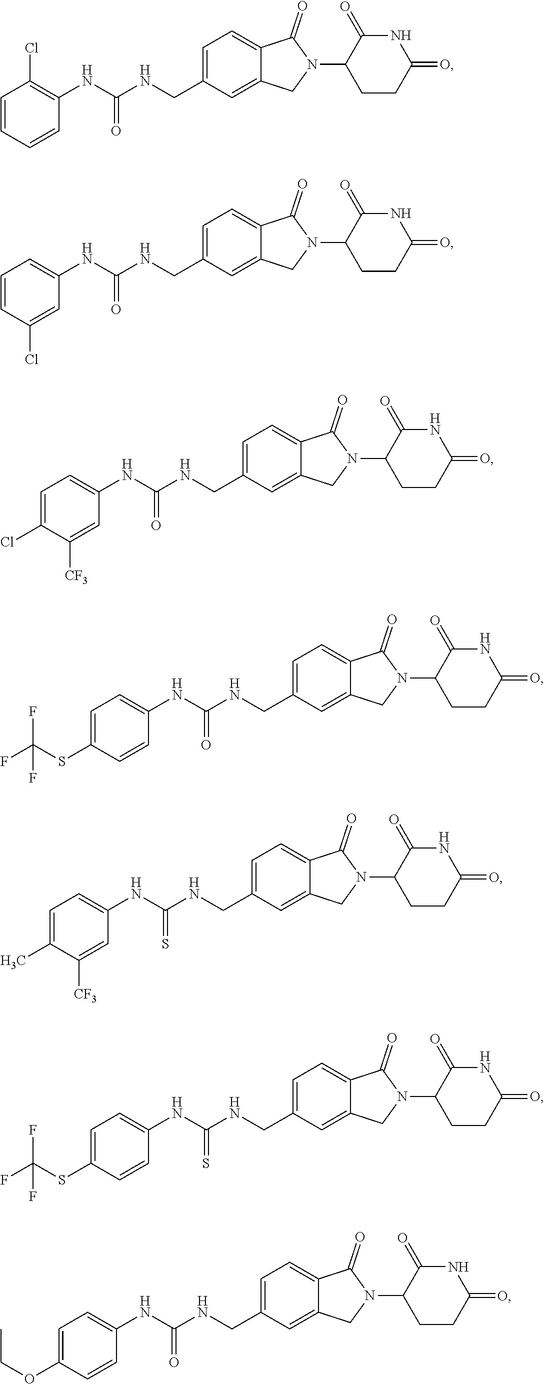

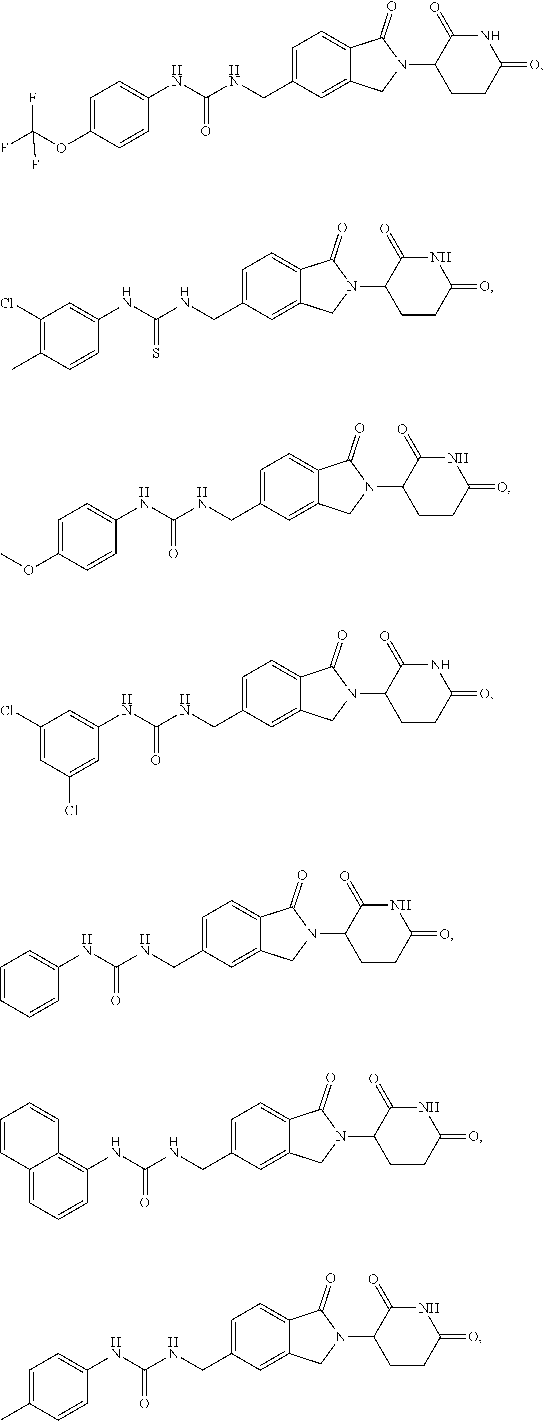

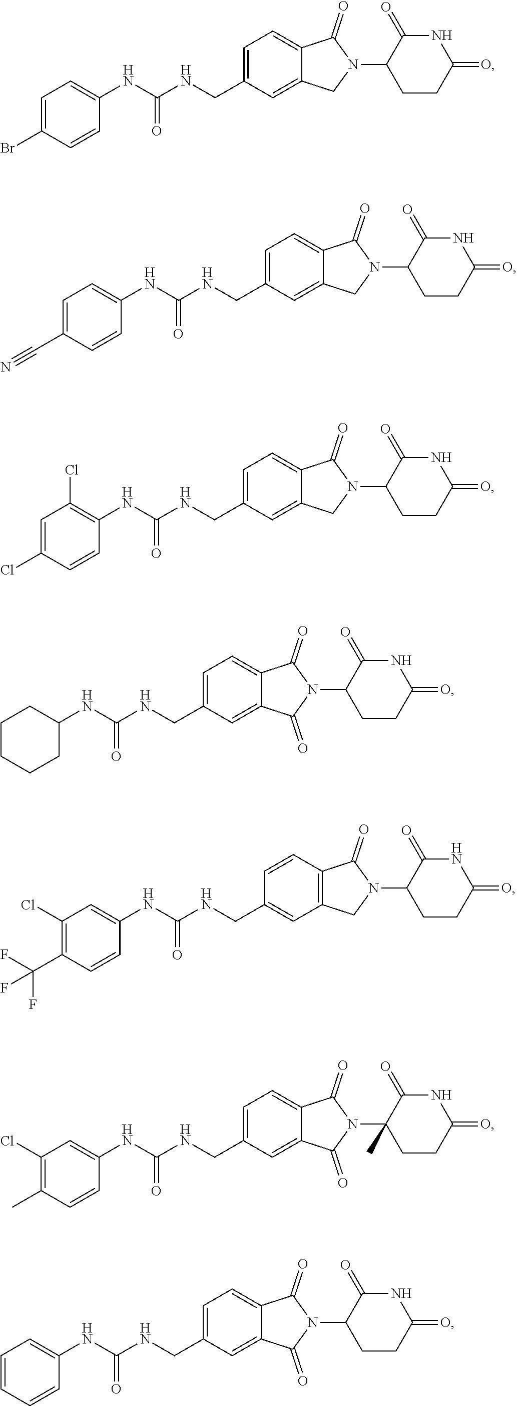

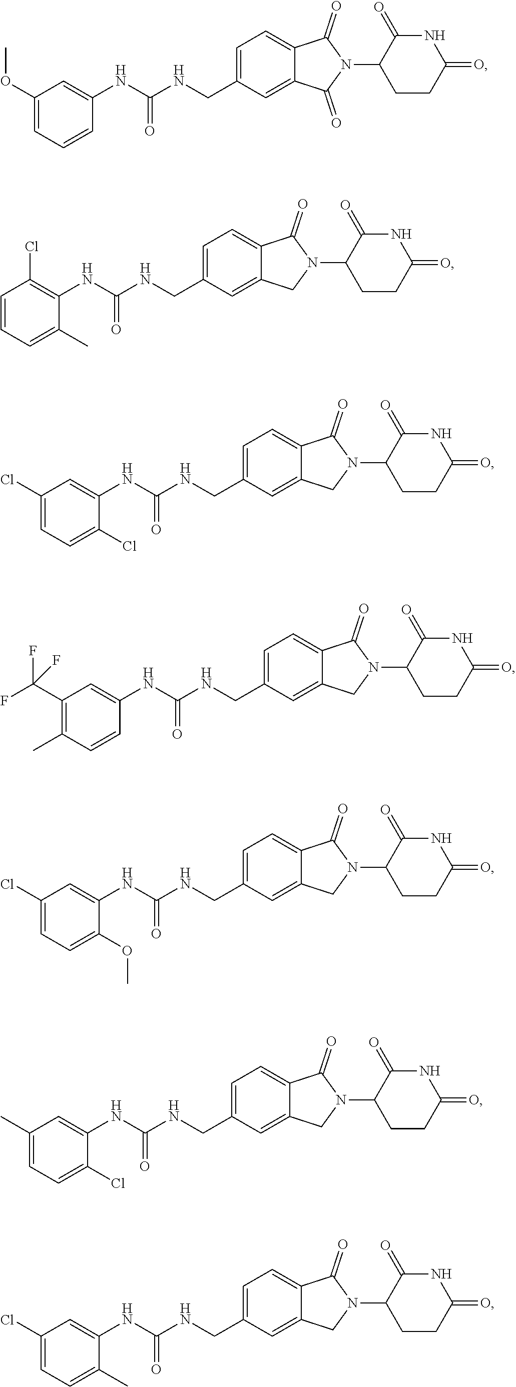

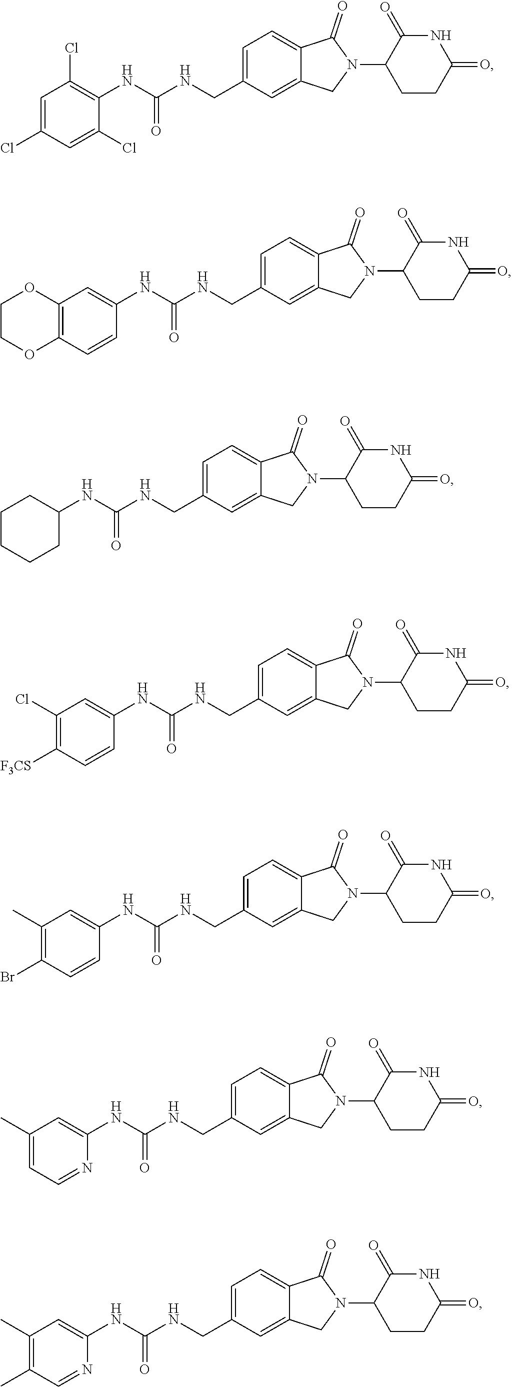

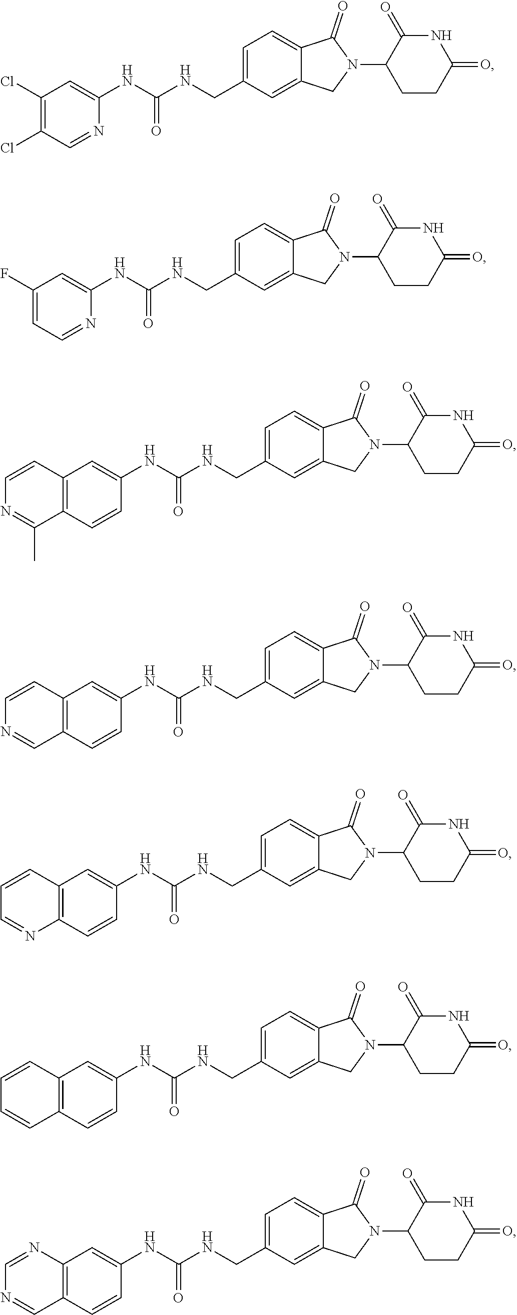

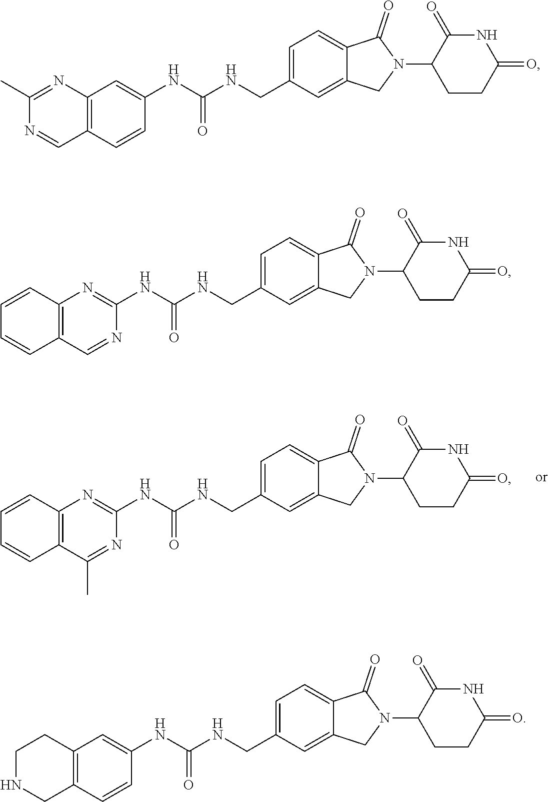 [SMILES: Cc1ncc2ccc(NC(=O)NCc3ccc4c(c3)CN(C3CCC(=O)NC3=O)C4=O)cc2n1 ; O=C1CCC(N2Cc3cc(CNC(=O)Nc4ncc5ccccc5n4)ccc3C2=O)C(=O)N1 ; Cc1nc(NC(=O)NCc2ccc3c(c2)CN(C2CCC(=O)NC2=O)C3=O)nc2ccccc12 ; "or" ; O=C1CCC(N2Cc3cc(CNC(=O)Nc4ccc5c(c4)CCNC5)ccc3C2=O)C(=O)N1]

In certain aspects, the immunomodulatory imide compound is Formula (b):

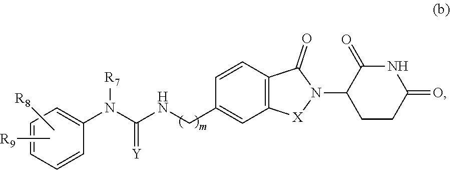

or a pharmaceutically acceptable salt, solvate, or stereoisomer thereof, wherein:
   X is C(═O) or CH₂;
   Y is O, cyanamido (N—≡N), or amido (NH);
   m is an integer of 0, 1, 2, or 3;
   R₇ is hydrogen or C₁₋₆ alkyl;
   R₈ is hydrogen, —NO₂, C₁-10 alkyl, C₀₋₆ alkyl-(5 to 10 membered heteroaryl), C₀₋₆ alkyl-(5 to 6 membered heterocyclyl), C₀₋₆ alkyl-OH, C₀₋₄ alkyl-NH₂, —NHCO—C₁₋₆ alkyl, —OR₂₁, or —(CH₂—Z)₀₋₂-(5 to 10 membered heteroaryl), where each heteroaryl and heterocyclyl is optionally substituted with one or more C₁₋₆ alkyl;
   R₉ is hydrogen, halogen, —NO₂, C₀₋₆ alkyl-(5 to 10 membered heteroaryl), C₀₋₆ alkyl-(5 to 6 membered heterocyclyl), C₀₋₆ alkyl-OH, C₀₋₄ alkyl-NH₂, —NHCO—C₁₋₆ alkyl, —OR₂₁, or —(CH₂—Z)₀₋₂-(5 to 10 membered heteroaryl), where each heteroaryl and heterocyclyl is optionally substituted with one or more C₁₋₆ alkyl;
   R₂₁ is C₆₋₁₀ aryl, 5 to 10 membered heteroaryl, 5 to 6 membered heterocyclyl, or —CO(CH₂)₀₋₂R₂₂, wherein the aryl, heteroaryl, and heterocyclyl are each optionally substituted with one or more C₁₋₆ alkyl;
   R₂₂ is —NH₂ or 5 to 6 membered heterocyclyl; and
   Z is CH₂, NH, or O.

In certain aspects, the structure of the immunomodulatory imide compound of Formula (b) is that when R₇ is hydrogen, then R₈ is not hydrogen or C_{1_6} alkyl; when Y is O, then R₉ is not halogen; and when Y is O and R₉ is halogen, then R₈ is C_{0_6} alkyl-(5-6 membered heterocyclyl).

In certain aspects, the 5′-substituted isoindoline compounds is Formula (c):

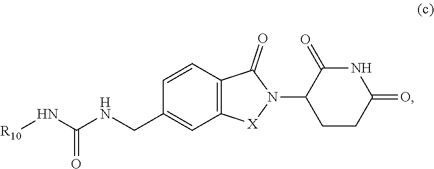

or a pharmaceutically acceptable salt, solvate, or stereoisomer thereof, wherein:
   X is C(═O) or CH₂;
   m is an integer of 0, 1, 2, or 3;
   R₁₀ is C₃₋₁₀ cycloalkyl, 5 to 10 membered heterocyclyl, 5 to 10 membered heteroaryl,
   or C₀₋₄ alkyl-NR₄₁R₄₂; wherein the cycloalkyl, heterocyclyl, and heteroaryl are each optionally
   substituted with one or more halogen, C₁₋₆ alkyl, —CO—NR₄₃R₄₄, —COOR₄₅, or C₀₋₄ alkyl-C₆₋₁₀ aryl,
   wherein the aryl itself may be optionally substituted with one or more halogen; and
   R₄₁, R₄₂, R₄₃, R₄₄, and R₄₅ are each independently hydrogen or C₁₋₆ alkyl.

In certain aspects, the 5′-substituted isoindoline compounds is Formula (d)

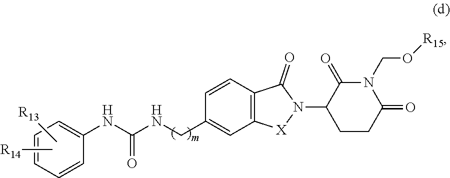

or a pharmaceutically acceptable salt, solvate, or stereoisomer thereof, wherein:
   X is C(═O) or CH₂;
   m is an integer of 0, 1, 2, or 3;
   R₁₃ and R₁₄ are each independently: hydrogen, halo, C₁₋₆ alkyl, oxo, —NO₂, —Z—C₀₋₆ alkyl-(5 to 10 membered heteroaryl), C₀₋₆ alkyl-(5 to 6 membered heterocyclyl), C₀₋₆ alkyl-OH, C₀₋₄ alkyl-NH₂, —NHCO—C₁₋₆ alkyl, —OR₂₁, or —(CH₂—Z)₀₋₂-(5 to 10 membered heteroaryl),
   wherein Z is S or SO₂;
   wherein R₂₁ is defined above;
   wherein each heteroaryl and heterocyclyl above is optionally substituted with one or more C₁₋₆ alkyl; and
   wherein the alkyl or alkoxy above may be optionally substituted with one or more: halogen; cyano; nitro; amino: C₁₋₆ alkylidenedioxy; C₁₋₆ alkoxy, itself optionally substituted with one or more halogens; or C₁₋₆ alkylthio, itself optionally substituted with one or more halogens:
   R₁₅ is COR₇₁ or PO(OR₇₂)(OR₇₃);
   R₇₁ is C₁₋₁₀ alkyl, C₆₋₁₀ aryl, or 5 to 6 membered heterocyclyl; wherein the alkyl, aryl, heterocyclyl may be optionally substituted with one or more amino, C₁₋₆ alkylamino, di(C₁₋₆ alkyl)amino, or —COOR₇₄; and
   R₇₂, R₇₃, and R₇₄ are each independently hydrogen or C₁₋₁₀ alkyl.

In certain aspects, the 5′-substituted isoindoline compounds is Formula (e)

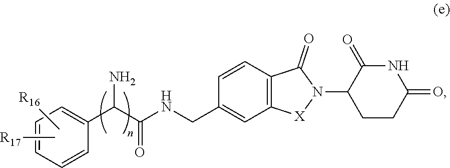

or a pharmaceutically acceptable salt, solvate, or stereoisomer thereof, wherein:
   X is C(═O) or CH:
   n is an integer of 0 or 1;
   R is hydrogen or halo; and
   R is hydrogen, amino, or 5 to 10 membered heteroaryl or heterocyclyl.

In certain aspects, the structure of the immunomodulatory imide compound of Formula (e) is that when m is 0, R₁₇ is not hydrogen.

In certain aspects, the immunomodulatory imide compound is Formula (f)

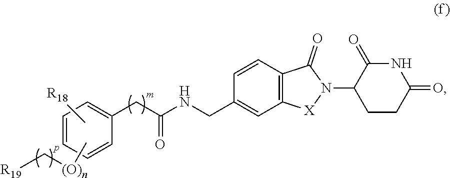

or a pharmaceutically acceptable salt, solvate, or stereoisomer thereof, wherein:
   X is CH or C═O;
   m and n are each independently 0 or 1;
   p is 0, 1, 2, or 3:
   R₁₉ is 5 to 6 membered heterocyclyl, optionally substituted with C₁₋₆ alkyl; and
   R₁₈ is hydrogen or halogen.

In certain aspects, the immunomodulatory imide compound is Formula (g)

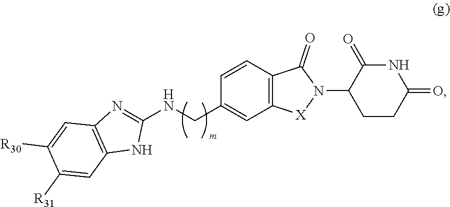

or a pharmaceutically acceptable salt, Solvate, prodrug, or stereoisomer thereof, wherein:
X is CH or C═O;
m is an integer of 0, 1, 2, or 3:

R₃₀ and R₃₁ are each independently hydrogen, halo, C₁₋₆ alkyl, or Caryloxy, wherein the alkyl and aryl are each optionally substituted with one or more halo.

### EXAMPLES

### Example 1: General Procedure for Preparation and Characterization of 5′-Substituted Isoindoline Conjugates

The 5′-substituted isoindoline compounds described in the present disclosure can be prepared by one of ordinary skill in the art following the procedures provided in International Publication Nos. WO 2008/027542, WO 2009/145899, WO 2010/053762. In light of the present disclosure and knowledge in the art, one of ordinary skill in the art would be able to synthesize a structure of linker-5′ substituted isoindoline (the structure of L′-V) using routine methods.

To conjugate an antibody to the structure of L′-V, a solution of antibody was treated with 30 equivalents of tris-(2-carboxyethyl)phosphine (TCEP) and incubated at 37° C. for 1 hour to reduce the interchain disulfides. The reduced antibody was purified into 50 mM EPPS, 5 mM EDTA pH 7.0 buffer using illustra NAP columns (GE Healthcare).

Conjugation was effected by treatment of a solution of reduced antibody at 2-5 mg/mL in 50 mM EPPS, 5 mM EDTA pH 7.0 with 12 equivalents of linker-5′ substituted isoindoline added as a stock solution in N,N-dimethylacetamide (DMA) such that the final concentration of DMA was 15% (v/v). The resulting reaction mixture was left overnight at 4° C. The resulting newDegrader conjugate was purified into 20 mM succinate, 8% sucrose, 0.01% Tween-20 pH 5.5 using illustra NAP columns (GE Healthcare) and concentrated using Amicon Ultra centrifugal concentrators with 50 kD molecular weight cutoff (Millipore).

Concentration and monomer were determined by size exclusion chromatography using a 7.8×300 mM TSKGel 3000SWXL column with 5 μm particles (Tosoh Bioscience), eluting isocratically with 400 mM sodium perchlorate, 50 mM sodium phosphate, 5% (v/v) isopropanol mobile phase running at 0.5 mg/mL for 30 min. 5′-substituted isoindoline conjugates were quantitated from antibody standard curves, detecting at 214 nm.

Drug to antibody ratio (DAR) was determined by hydrophobic interaction chromatography using a 4.6×35 mm TSKgel Butyl-NPR column with 2.5 μm particles. Mobile phase A was 1.5 M ammonium sulfate, 25 mM sodium phosphate pH 7.0. Mobile phase B was 25 mM sodium phosphate pH 7.0, 25% (v/v) isopropanol. Analytes were eluted with a linear gradient of 0-100% B in 12 min. at a flow rate of 0.6 mL/min. Detection was at 214 nm.

Free linker-payload was determined by mixed-mode chromatography using a 4.6×250 mm HISEP column with 2.5 μm particles (Supelco). Mobile phase A was 100 mM ammonium acetate. Mobile phase B was 100% acetonitrile. Analytes were eluted with a gradient of 25-40% B in 25 min., then 40-100% B in 2 min at a flow rate of 0.7 mL/min. Column temperature was 35° C. Free linker-payload was quantitated using an external standard curve, detecting at 254 nm.

### Example 2: General Procedure for In Vitro Antiproliferation Assay for 5-Substituted Isoindoline Conjugates

The ability of 5′-substituted isoindoline conjugates to inhibit cell growth was measured using in vitro anti-proliferation assay. Target cells were plated at 1,500-5,000 cells per well in 100 μL complete cell growth medium (RPMI 1640, 10% fetal bovine serum and 1% Penicillin-streptomycin for most cell lines; Hybri-care medium, 1.5 g/L sodium bicarbonate, 10% fetal bovine serum and 1% Penicillin-streptomycin for BT-474; RPMI 1640, 20% fetal bovine serum and 1% Penicillin-streptomycin for HL-60). Conjugates were diluted in complete cell growth medium using 4-fold serial dilutions and 100 μL was added per well. The final concentration typically ranged from 1×10⁻⁸ M to 1.53×10⁻¹³ M or 1×10⁻⁷ M to 1.53×10⁻¹² M. Cells were incubated at 37° C. in a humidified 5% CO₂ incubator for 5 days. Viability of remaining cells was determined by colorimetric WST-8 assay (Dojindo Molecular Technologies, Inc., Rockville, MD, US). WST-8 was added to 10% of the final volume and plates were incubated at 37° C. in a humidified 5% CO₂ incubator for 2-4 hours. Plates were analyzed by measuring the absorbance at 450 nm (A450) in a multi-well plate reader. Background A450 absorbance of wells with media and WST-8 only was subtracted from all values. The percent viability was calculated by dividing each treated sample value by the average value of wells with untreated cells. The percent viability value was plotted against the test sample concentration in a semi-log plot for each treatment. IC50 values were calculated automatically.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A conjugate comprising a binding moiety that specifically binds to a protein, and an immunomodulatory imide compound, wherein the binding moiety and the immunomodulatory imide compound are linked via an optional linker (L).

2. The conjugate of claim 1, wherein the conjugate has formula (I): 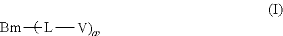
or a pharmaceutically acceptable salt thereof, wherein:
a is an integer from 1 to 10;
V is a substituted isoindoline compound;
L is a linker; and
Bm is a binding moiety that is capable of specifically binding to a protein.

3. The conjugate of claim 2, wherein V is a 5′ substituted isoindoline compound.

4. The conjugate of any one of claims 1 to 3, wherein the binding moiety is an antibody, antibody fragment, or an antigen-binding fragment.

5. The conjugate of any one of claims 1 to 4, or a pharmaceutically acceptable salt thereof, wherein a is 6, 7, or 8.

6. The conjugate of any one of claims 1 to 5, or a pharmaceutically acceptable salt thereof, wherein L is a non-cleavable linker.

7. The conjugate of claim 6, or a pharmaceutically acceptable salt thereof, wherein L is selected from the group consisting of 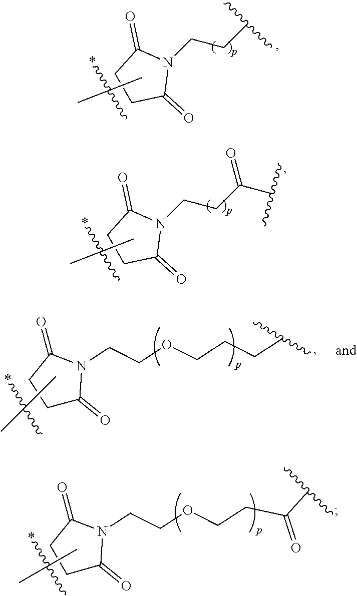
wherein:
p is an integer from 1 to 10; [symbol]
is the point of attachment to X; and [symbol]
is the point of attachment to the binding moiety.

8. The conjugate of claim 7, or a pharmaceutically acceptable salt thereof, wherein L is

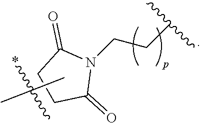

9. The conjugate of claim 8, or a pharmaceutically acceptable salt thereof, wherein p is 5.

10. The conjugate of any one of claims 1 to 5, or a pharmaceutically acceptable salt thereof, wherein L is a cleavable linker.

11. The conjugate of claim 10, or a pharmaceutically acceptable salt thereof, wherein the cleavable linker is cleavable by a protease.

12. The conjugate of claim 10 or 11, or a pharmaceutically acceptable salt thereof, wherein L is selected from the group consisting of 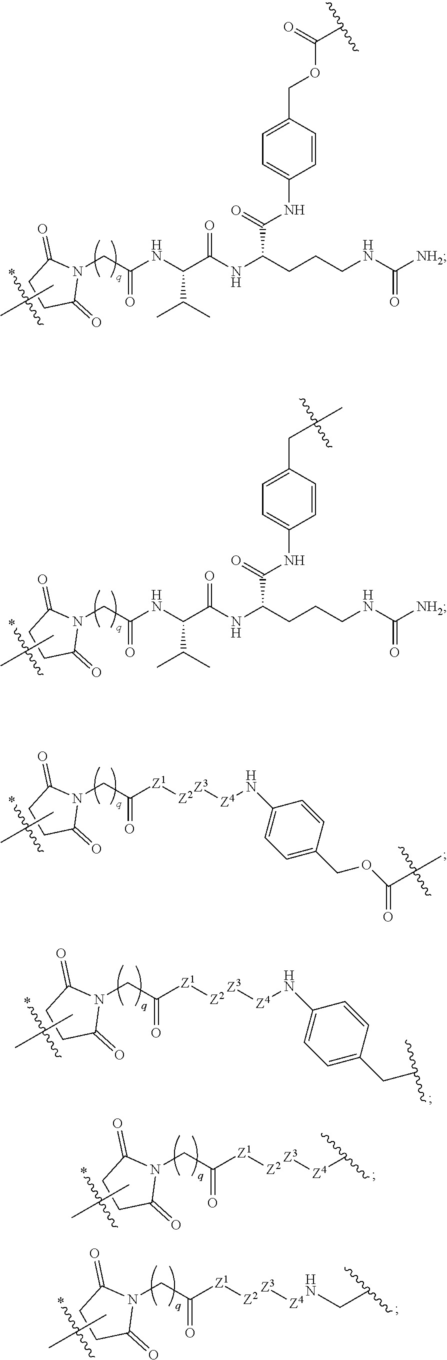 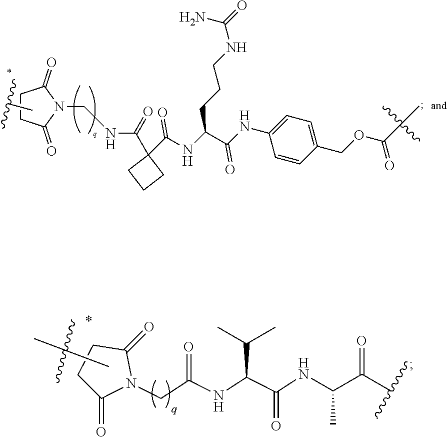
wherein:
q is an integer from 2 to 10;
Z¹, Z², Z³, and Z⁴ are each independently absent or a naturally-occurring amino acid residue in the L- or D-configuration, provided that at least two of Z¹, Z², Z³, and Z⁴ are amino acid residues; [symbol]
is the point of attachment to X; and [symbol]
is the point of attachment to the binding moiety.

13. The conjugate of claim 12, or a pharmaceutically acceptable salt thereof, wherein Z¹, Z², Z³, and Z⁴ are independently absent or selected from the group consisting of L-valine, D-valine, L-citrulline, D-citrulline, L-alanine, D-alanine, L-glutamine, L-glutamic acid, D-glutamic acid, D-glutaimine, L-aspartic acid, D-aspartic acid, L-asparagine, D-asparagine, L-phenylalanine, D-phenylalanine, L-lysine, D-lysine, and glycine; provided that at least two of Z¹, Z², Z³, and Z⁴ are amino acid residues.

14. The conjugate of claim 13, or a pharmaceutically acceptable salt thereof, wherein:
Z¹ is absent or glycine;
Z² is absent or selected from the group consisting of L-glutamine, L-glutamic acid, D-glutamic acid, D-glutamine, L-aspartic acid, D-aspartic acid, L-alanine, D-alanine, and glycine;
Z³ is selected from the group consisting of L-valine, D-valine, L-alanine, D-alanine, L-phenylalanine, D-phenylalanine, and glycine; and
Z⁴ is selected from the group consisting of L-alanine, D-alanine, L-citrulline, D-citrulline, L-asparagine, D-asparagine, L-lysine, D-lysine, L-phenylalanine, D-phenylalanine, and glycine.

15. The conjugate of claim 12, or a pharmaceutically acceptable salt thereof, wherein L is

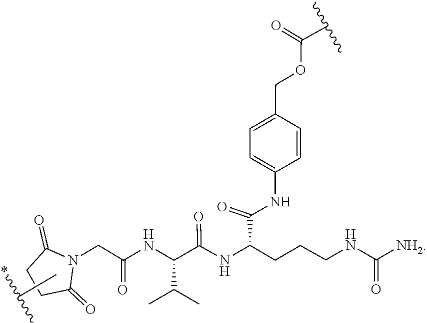

16. The conjugate of claim 15, or a pharmaceutically acceptable salt thereof, wherein q is 5.

17. The conjugate of claim 10, or a pharmaceutically acceptable salt thereof, wherein L is a bioreducible linker.

18. The conjugate of claim 10 or 17, wherein L is selected from the group consisting of 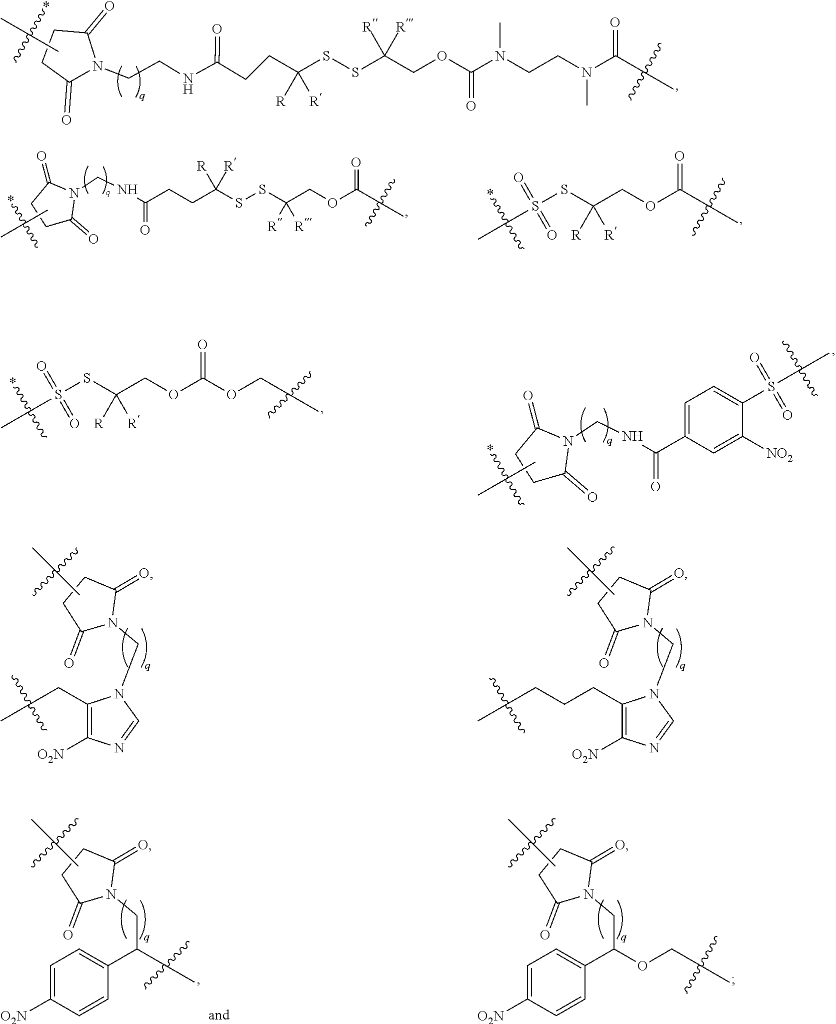
wherein:
q is an integer from 2 to 10;
R, R′, R″, and R′″ are each independently selected from hydrogen, C₁-C₆alkoxyC₁-C₆alkyl, (C₁-C₆)₂NC₁—C₆alkyl, and C₁-C₆alkyl or, two geminal R groups, together with the carbon atom to which they are attached, can form a cyclobutyl or cyclopropyl ring; [symbol]
is the point of attachment to X; and [symbol]
is the point of attachment to the binding moiety.

19. The conjugate of claim 10, or a pharmaceutically acceptable salt thereof, wherein L is an acid cleavable linker.

20. The conjugate of claim 10 or 19, or a pharmaceutically acceptable salt thereof, wherein L is selected from the group consisting of 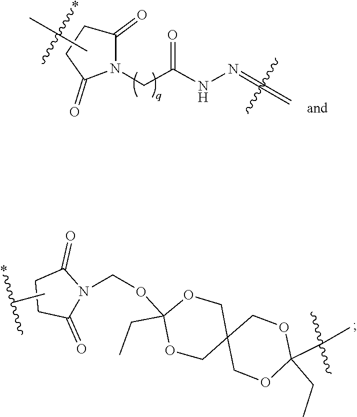
wherein:
q is an integer from 2 to 10; [symbol]
is the point of attachment to X; and [symbol]
is the point of attachment to the binding moiety.

21. The conjugate of claim 10, or a pharmaceutically acceptable salt thereof, wherein L is a click-to-release linker.

22. The conjugate of claim 10 or 21, or a pharmaceutically acceptable salt thereof, wherein L is selected from 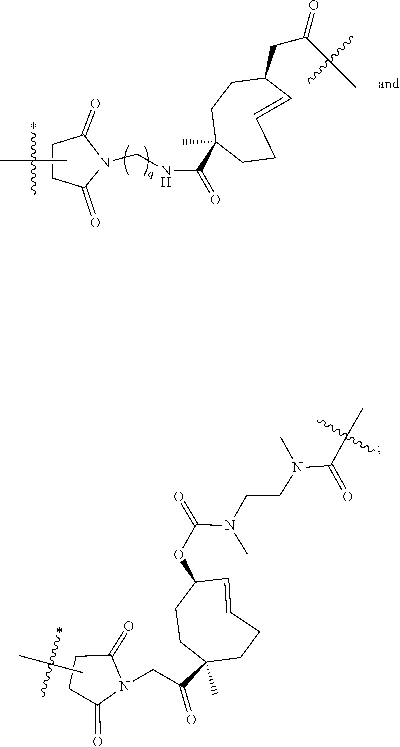
wherein:
q is an integer from 2 to 10; [symbol]
is the point of attachment to X; and [symbol]
is the point of attachment to the binding moiety.

23. The conjugate of claim 10, or a pharmaceutically acceptable salt thereof, wherein L is a pyrophosphatase cleavable linker.

24. The conjugate of claim 23, or a pharmaceutically acceptable salt thereof, wherein L is 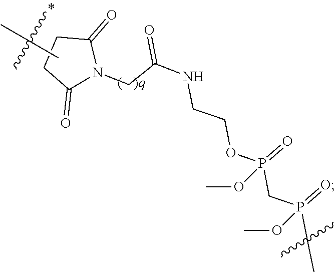
wherein:
q is an integer from 2 to 10; [symbol]
is the point of attachment to X; and [symbol]
is the point of attachment to the binding moiety.

25. The conjugate of claim 10, or a pharmaceutically acceptable salt thereof, wherein L is a beta-glucoronidase cleavable linker.

26. The conjugate of claim 10 or claim 25, or a pharmaceutically acceptable salt thereof, wherein L is selected from 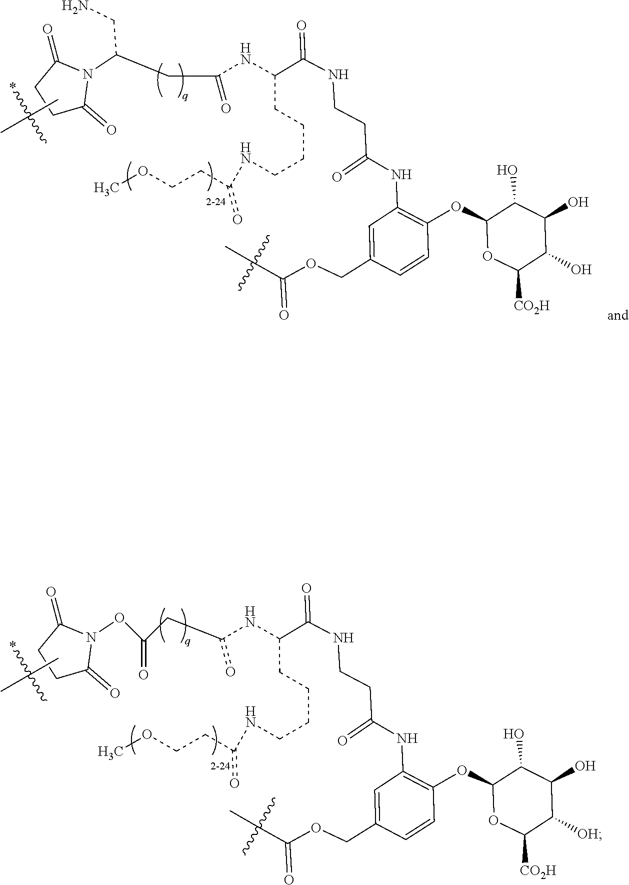
wherein:
q is an integer from 2 to 10;
---- is absent or a bond; [symbol]
is the point of attachment to X; and [symbol]
is the point of attachment to the binding moiety.

27. The conjugate of any one of claims 1 to 26, or a pharmaceutically acceptable salt thereof, wherein Bm is an antibody or antigen binding portion thereof.

28. The conjugate of claim 27, wherein the protein that the binding moiety binds to is a surface antigen.

29. The conjugate of claim 28, wherein the surface antigen comprises 5T4, ACE, ADRB3, AKAP-4, ALK, Androgen receptor, AOC3, APP, Axin1, AXL, B7H3, B7-H4, BCL2, BCMA, bcr-abl, BORIS, BST2, C242, C4.4a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudine 3, CLDN6, CLEC12A, CLL-1, cll3, c-MET, Crypto 1 growth factor, CS1, CTLA-4, CXCR2, CXORF61, Cyclin B1, CYP1B1, Cadherin-3, Cadherin-6, DLL3, E7, EDNRB, EFNA4, EGFR, EGFRvIII, ELF2M, EMR2, ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GloboH, GM3, GPC1, GPC2, GPC3, gplOO, GPNMB, GPR20, GPRC5D, GUCY2C, HAVCR1, HER2, HER3, HGF, HMI.24, HMWMAA, HPV E6, hTERT, human telomerase reverse transcriptase, ICAM, ICOS-L, IFN-α, IFN-γ, IGF-I receptor, IGLL1, IL-2 receptor, IL-4 receptor, IL-13Ra2, IL-l lRa, IL-1, IL-12, IL-23, IL-13, IL-22, IL-4, IL-5, IL-6, interferon receptor, integrins (including α₄, αᵥβ₃, αᵥβ₅, αᵥβ₆, α₁β₄, α₄β₁, α₄β₇, α₅β₁, α₆β₄, α_{IIb}β₃ integrins), Integrin alphaV, intestinal carboxyl esterase, KIT, LAGE-la, LAIR1, LAMP-1, LCK, Legumain, LewisY, LFA-1(CD11a), L-selectin(CD62L), LILRA2, LIV-1, LMP2, LRRC15, LY6E, LY6K, LY75, MAD-CT-1, MAD-CT-2, MAGE Al, MelanA/MARTl, Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2, OY-TES1, p53, p53 mutant, PANX3, PAP, PAX3, PAX5, p-CAD, PCTA-1/Galectin 8, PD-L1, PD-L2, PDGFR, PDGFR-beta, phosphatidylserine, PIK3CA, PLAC1, Polysialic acid, Prostase, prostatic carcinoma cell, prostein, Pseudomonas aeruginosa, rabies, survivin and telomerase, PRSS21, PSCA, PSMA, PTK7, RAGE-1, RANKL, Ras mutant, respiratory syncytial virus, Rhesus factor, RhoC, RON, ROR1, ROR2, RU1, RU2, sarcoma translocation breakpoints, SART3, SLAMF7, SLC44A4, sLe, SLITRK6, sperm protein 17, sphingosine-I-phosphate, SSEA-4, SSX2, STEAP1, TAG72, TARP, TCRβ, TEM1/CD248, TEM7R, tenascin C, TF, TGF-1, TGF-β2, TNF-α, TGS5, Tie 2, TIM-1, Tn Ag, TRAC, TRAIL-R1, TRAIL-R2, TROP-2, TRP-2, TRPV1, TSHR, tumor antigen CTAA16.88, tyrosinase, UPK2, VEGF, VEGFR1, VEGFR2, vimentin, WTl, XAGE1, or combinations thereof.

30. The conjugate of claim 27, or a pharmaceutically acceptable salt thereof, wherein the surface antigen comprises HER2, CD20, CD38, CD33, BCMA, CD138, EGFR, FGFR4, GD2, PDGFR, TEM1/CD248, TROP-2, or combinations thereof.

31. The conjugate of claim 27, or a pharmaceutically acceptable salt thereof, wherein the antibody is selected from the group consisting of rituximab, trastuzumab, gemtuzumab, pertuzumab, obinutuzumab, ofatumumab, olaratumab, ontuximab, isatuximab, Sacituzumab, U3-1784, daratumumab, STI-6129, lintuzumab, huMy9-6, balantamab, indatuximab, cetuximab, dinutuximab, anti-CD38 A2 antibody, huAT13/5 antibody, alemtuzumab, ibritumomab, tositumomab, bevacizumab, panitumumab, tremelimumab, ticilimumab, catumaxomab, oregovomab, and veltuzumab.

32. The conjugate of claim 31, or a pharmaceutically acceptable salt thereof, wherein the antibody is rituximab, trastuzumab, pertuzumab, huMy9-6, lintuzumab, or gemtuzumab.

33. The conjugate of any one of claims 2 to 32, or a pharmaceutically acceptable salt thereof, wherein V has Formula (a): 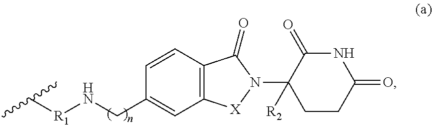
or a pharmaceutically acceptable salt, solvate, or stereoisomer thereof, wherein:
n is 0 or 1;
X is CH₂, C═O, or C═S;
R₁ is:
a) —(CH₂)ₘR₃ or —CO(CH₂)ₘR₃, wherein
m is 0, 1, 2, or 3; and
R₃ is 5-10 membered aryl or heteroaryl, optionally substituted with one or more halogen;
b) —C═YR₄, wherein
Y is O or S; and
R₄ is:
(C₀-C₁₀)alkyl-(5 to 10 membered heteroaryl or heterocycle), said heteroaryl or heterocycle optionally substituted with one or more of (C₁-C₆)alkyl, halogen, oxo, (C₁-C₆)alkoxy, or —Z—(C₁-C₆)alkyl, wherein Z is S or SO₂, and wherein said (C₁-C₆)alkyl may be optionally substituted with one or more halogen;
(C₀-C₁₀)alkyl-(5 to 10 membered aryl), said aryl optionally substituted with one or more of: halogen; (C₁-C₆)alkoxy, itself optionally substituted with one or more halogen; (C₁-C₆)alkyl, itself optionally substituted with one or more halogen; or —Z—(C₁-C₆)alkyl, wherein Z is S or SO₂, and wherein said (C₁-C₆)alkyl may be optionally substituted with one or more halogen; or (C₁-C₆)alkyl-CO—O—R₁₂, wherein R₁₂ is H or (C₁-C₆)alkyl; or
c) —C═ZNHR₆, wherein
Z is O or S; and
R₆ is:
5 to 10 membered aryl or heteroaryl, optionally substituted with one or more of: halogen; cyano; (C₁-C₆)alkylenedioxy; (C₁-C₅)alkoxy, itself optionally substituted with one or more halogen; (C₁-C₆)alkyl, itself optionally substituted with one or more halogen; or (C₁-C₆)alkylthio, itself optionally substituted with one or more halogen; and
R₂ is H or (C₁-C₆)alkyl.

34. The conjugate of claim 33, or a pharmaceutically acceptable salt, solvate, or stereoisomer thereof, wherein:
n is 0 or 1;
X is CH₂ or C═O;
R₁ is —(CH₂)ₘR₃, wherein
m is 0, 1, 2, or 3; and
R₃ is 5-10 membered aryl or heteroaryl, optionally substituted with one or more halogen.

35. The conjugate of claim 33, or a pharmaceutically acceptable salt, solvate, or stereoisomer thereof, wherein:
n is 1;
X is CH₂ or C═O;
R₁ is:
C═OR₄ or —C═SR₄, wherein
R₄ is:
(C₀-C₁₀)alkyl-(5 to 10 membered heteroaryl or heterocycle), said heteroaryl or heterocycle optionally substituted with one or more of (C₁-C₆)alkyl, halogen, oxo, (C₁-C₆)alkoxy, or —Z—(C₁-C₆)alkyl, wherein Z is S or SO₂, and wherein said (C₁-C₆)alkyl may be optionally substituted with one or more halogen;
(C₀-C₁₀)alkyl-(5 to 10 membered aryl), said aryl optionally substituted with one or more of: halogen; (C₁-C₆)alkoxy, itself optionally substituted with one or more halogen; (C₁-C₆)alkyl, itself optionally substituted with one or more halogen; or —Z—(C₁-C₆)alkyl, wherein Z is S or SO₂, and wherein said (C₁-C₆)alkyl may be optionally substituted with one or more halogen; or (C₁-C₆)alkyl-CO—O—R₁₂, wherein R₁₂ is H or (C₁-C₆)alkyl.

36. The conjugate of claim 33, or a pharmaceutically acceptable salt, solvate, or stereoisomer thereof, wherein:
n is 1;
X is CH₂ or C═O;
R₁ is:
C═ONHR₆ or —C═SNHR₆, wherein
R₆ is:
5 to 10 membered aryl or heteroaryl, optionally substituted with one or more of: halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen.

37. The conjugate of any one of claims 2 to 32, or a pharmaceutically acceptable salt, solvate, or stereoisomer thereof, wherein V has Formula (b): 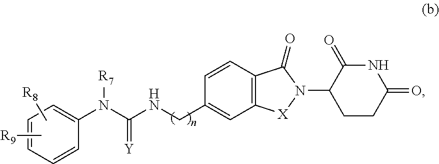
or a pharmaceutically acceptable salt, solvate, or stereoisomer thereof, wherein:
X is C(═O) or CH₂;
Y is O, cyanamido (N—≡N), or amido (NH);
m is an integer of 0, 1, 2, or 3;
R₇ is hydrogen or C₁₋₆ alkyl;
R₈ is hydrogen, —NO₂, C₁₋₁₀ alkyl, C₀₋₆ alkyl-(5 to 10 membered heteroaryl), C₀₋₆ alkyl-(5 to 6 membered heterocyclyl), C₀₋₆ alkyl-OH, C₀₋₄ alkyl-NH₂, —NHCO—C₁₋₆ alkyl, —OR₂₁, or —(CH₂—Z)₀₋₂-(5 to 10 membered heteroaryl), where each heteroaryl and heterocyclyl is optionally substituted with one or more C₁₋₆ alkyl;
R₉ is hydrogen, halogen, —NO₂, C₀₋₆ alkyl-(5 to 10 membered heteroaryl), C₀₋₆ alkyl-(5 to 6 membered heterocyclyl), C₀₋₆ alkyl-OH, C₀₋₄ alkyl-NH₂, —NHCO—C₁₋₆ alkyl, —OR₂₁, or —(CH₂—Z)₀₋₂-(5 to 10 membered heteroaryl), where each heteroaryl and heterocyclyl is optionally substituted with one or more C₁₋₆ alkyl;
R₂₁ is C₆₋₁₀ aryl, 5 to 10 membered heteroaryl, 5 to 6 membered heterocyclyl, or —CO(CH₂)₀₋₂R₂₂, wherein the aryl, heteroaryl, and heterocyclyl are each optionally substituted with one or more C₁₋₆ alkyl;
R₂₂ is —NH₂ or 5 to 6 membered heterocyclyl; and
Z is CH₂, NH, or O.

38. The conjugate of claim 37, or a pharmaceutically acceptable salt, solvate, or stereoisomer thereof, wherein when R₇ is hydrogen, then R₈ is not hydrogen or C_{1_6} alkyl; when Y is O, then R₉ is not halogen; and when Y is O and R₉ is halogen, then R₈ is C_{0_6} alkyl-(5-6 membered heterocyclyl).

39. The conjugate of any one of claims 2 to 32, or a pharmaceutically acceptable salt, solvate, or stereoisomer thereof, wherein V has Formula (c): 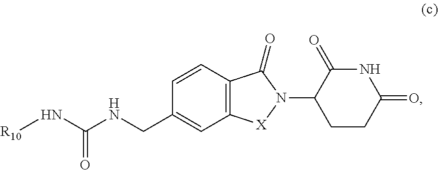
or a pharmaceutically acceptable salt, solvate, or stereoisomer thereof, wherein:
X is C(═O) or CH₂;
m is an integer of 0, 1, 2, or 3;
R₁₀ is C₃₋₁₀ cycloalkyl, 5 to 10 membered heterocyclyl, 5 to 10 membered heteroaryl,
or C₀₋₄ alkyl-NR₄₁R₄₂; wherein the cycloalkyl, heterocyclyl, and heteroaryl are each optionally substituted with one or more halogen, C₁₋₆ alkyl, —CO—NR₄₃R₄₄, —COOR₄₅, or C₀₋₄ alkyl-C₆₋₁₀ aryl,
wherein the aryl itself may be optionally substituted with one or more halogen; and
R₄₁, R₄₂, R₄₃, R₄₄, and R₄₅ are each independently hydrogen or C₁₋₆ alkyl.

40. The conjugate of any one of claims 2 to 32, or a pharmaceutically acceptable salt, solvate, or stereoisomer thereof, wherein V has Formula (d) 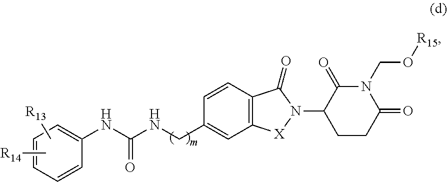
or a pharmaceutically acceptable salt, solvate, or stereoisomer thereof, wherein:
X is C(═O) or CH₂;
m is an integer of 0, 1, 2, or 3;
R₁₃ and R₁₄ are each independently: hydrogen, halo, C₁₋₆ alkyl, oxo, —NO₂, C₁₋₆ alkoxy, —Z—C₀₋₆ alkyl, C₀₋₆ alkyl -(5 to 10 membered heteroaryl), C₀₋₆ alkyl-(5 to 6 membered heterocyclyl), C₀₋₆ alkyl-OH, C₀₋₄ alkyl-NH₂, —NHCO—C₁₋₆ alkyl, —OR₂₁, or —(CH₂—Z)₀₋₂-(5 to 10 membered heteroaryl),
wherein Z is S or SO₂;
wherein R₂₁ is defined above;
wherein each heteroaryl and heterocyclyl above is optionally substituted with one or more C₁₋₆ alkyl; and
wherein the alkyl or alkoxy above may be optionally substituted with one or more: halogen; cyano; nitro; amino: C₁₋₆ alkylidenedioxy; C₁₋₆ alkoxy, itself optionally substituted with one or more halogens; or C₁₋₆ alkylthio, itself optionally substituted with one or more halogens:
R₁₅ is COR₇₁ or PO(OR₇₂)(OR₇₃);
R₇₁ is C₁₋₁₀ alkyl, C₆₋₁₀ aryl, or 5 to 6 membered heterocyclyl; wherein the alkyl, aryl, heterocyclyl may be optionally substituted with one or more amino, C₁₋₆ alkylamino, di(C₁₋₆ alkyl)amino, or —COOR₇₄; and
R₇₂, R₇₃, and R₇₄ are each independently hydrogen or C₁₋₁₀ alkyl.

41. The conjugate of any one of claims 2 to 32, or a pharmaceutically acceptable salt, solvate, or stereoisomer thereof, wherein V has Formula (e) 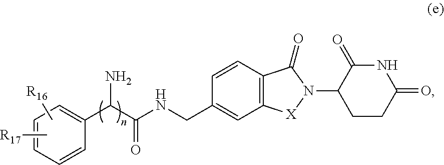
or a pharmaceutically acceptable salt, solvate, or stereoisomer thereof, wherein:
X is C(═O) or CH:
n is an integer of 0 or 1;
R₁₆ is hydrogen or halo; and
R₁₇ is hydrogen, amino, or 5 to 10 membered heteroaryl or heterocyclyl.

42. The conjugate of claim 41, or a pharmaceutically acceptable salt, solvate, or stereoisomer thereof, wherein when m is 0, R₁₇ is not hydrogen.

43. The conjugate of any one of claims 2 to 32, or a pharmaceutically acceptable salt, solvate, or stereoisomer thereof, wherein V has Formula (f) 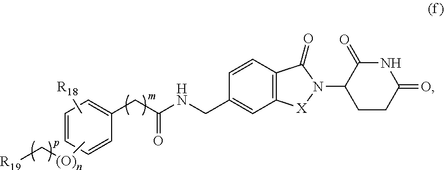
or a pharmaceutically acceptable salt, solvate, or stereoisomer thereof, wherein:
X is CH or C═O;
m and n are each independently 0 or 1;
p is 0, 1, 2, or 3:
R₁₉ is 5 to 6 membered heterocyclyl, optionally substituted with C₁₋₆ alkyl; and
R₁₈ is hydrogen or halogen.

44. The conjugate of any one of claims 2 to 32, or a pharmaceutically acceptable salt, solvate, or stereoisomer thereof, wherein V has Formula (g) 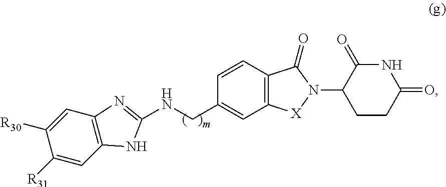
or a pharmaceutically acceptable salt, solvate, prodrug, or stereoisomer thereof, wherein:
X is CH or C═O;
m is an integer of 0, 1, 2, or 3:
R₃₀ and R₃₁ are each independently hydrogen, halo, C₁₋₆ alkyl, or C₁₋₁₀aryloxy, wherein the alkyl and aryl are each optionally substituted with one or more halo.

45. A method of preparing the conjugate of any one of claims 1 to 44, or a pharmaceutically acceptable salt thereof, the process comprising reacting a binding moiety with a structure L′-V, wherein
V is a 5-substituted isoindoline compound;
L′ is a cleavable or non-cleavable linker precursor that conjugates to the binding moiety.

46. The method of claim 45, further comprising reducing the binding moiety prior to reacting with the structure L′-V.

47. The method of claim 45 or 46, wherein a is 6, 7, or 8.

48. The method of any one of claims 45 to 47, wherein L′ is a non-cleavable linker precursor.

49. The method of claim 48, wherein L′ is selected from the group consisting of 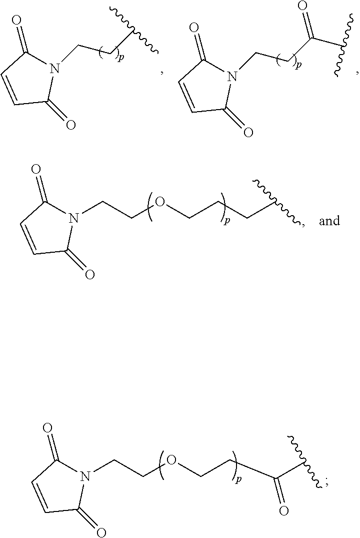
wherein:
p is an integer from 1 to 10; and [symbol]
is the point of attachment to X.

50. The method of claim 49, wherein L′ is

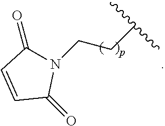

51. The method of claim 50, wherein p is 5.

52. The method of any one of claims 45 to 48, wherein L′ is a cleavable linker precursor.

53. The method of claim 52, wherein the cleavable linker precursor is cleavable by a protease.

54. The method of claim 52 or 53, wherein L′ is selected from the group consisting of 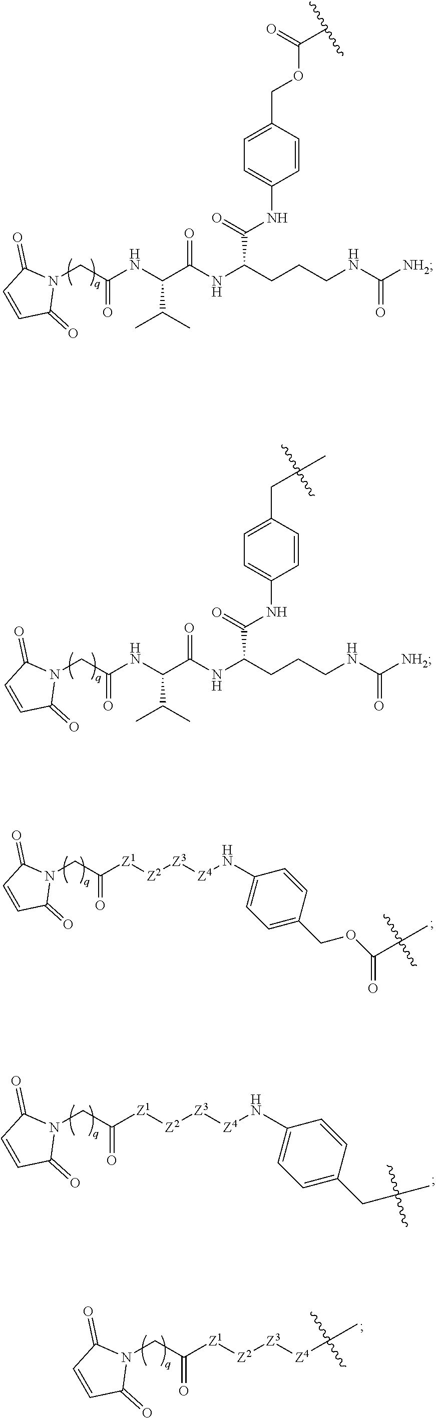 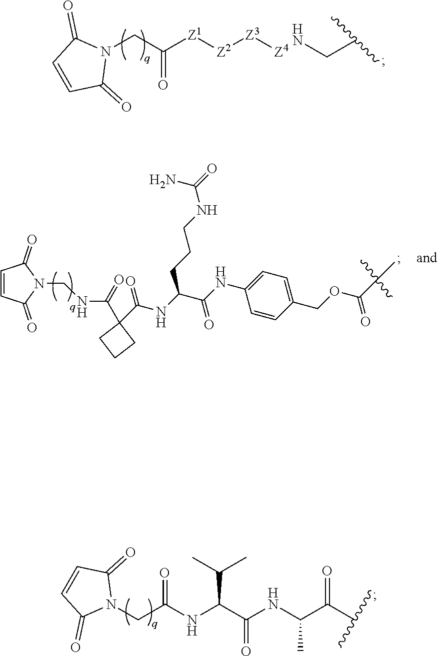
wherein:
q is an integer from 2 to 10;
Z¹, Z², Z³, and Z⁴ are each independently absent or a naturally-occurring amino acid residue in the L- or D-configuration, provided that at least two of Z¹, Z², Z³, and Z⁴ are amino acid residues; and [symbol]
is the point of attachment to X.

55. The method of claim 54, wherein Z¹, Z², Z³, and Z⁴ are independently absent selected from the group consisting of L-valine, D-valine, L-citrulline, D-citrulline, L-alanine, D-alanine, L-glutamine, L-glutamic acid, D-glutamic acid, D-glutaimine, L-aspartic acid, D-aspartic acid, L-asparagine, D-asparagine, L-phenylalanine, D-phenylalanine, L-lysine, D-lysine, and glycine, provided that at least two of Z¹, Z², Z³, and Z⁴ are amino acid residues.

56. The method of claim 55, wherein:
Z¹ is absent or glycine;
Z² is absent or selected from the group consisting of L-glutamine, L-glutamic acid, D-glutamic acid, D-glutamine, L-aspartic acid, D-aspartic acid, L-alanine, D-alanine, and glycine;
Z³ is selected from the group consisting of L-valine, D-valine, L-alanine, D-alanine, L-phenylalanine, D-phenylalanine, and glycine; and
Z⁴ is selected from the group consisting of L-alanine, D-alanine, L-citrulline, D-citrulline, L-asparagine, D-asparagine, L-lysine, D-lysine, L-phenylalanine, D-phenylalanine, and glycine.

57. The method of claim 54, wherein L′ is

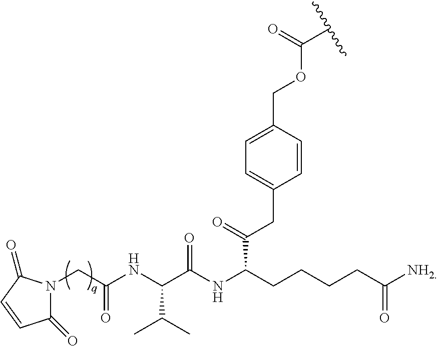

58. The method of claim 57, wherein q is 5.

59. The method of claim 52, wherein L′ is a bioreducible linker precursor.

60. The method of claim 52 or 59, wherein L′ is selected from the group consisting of 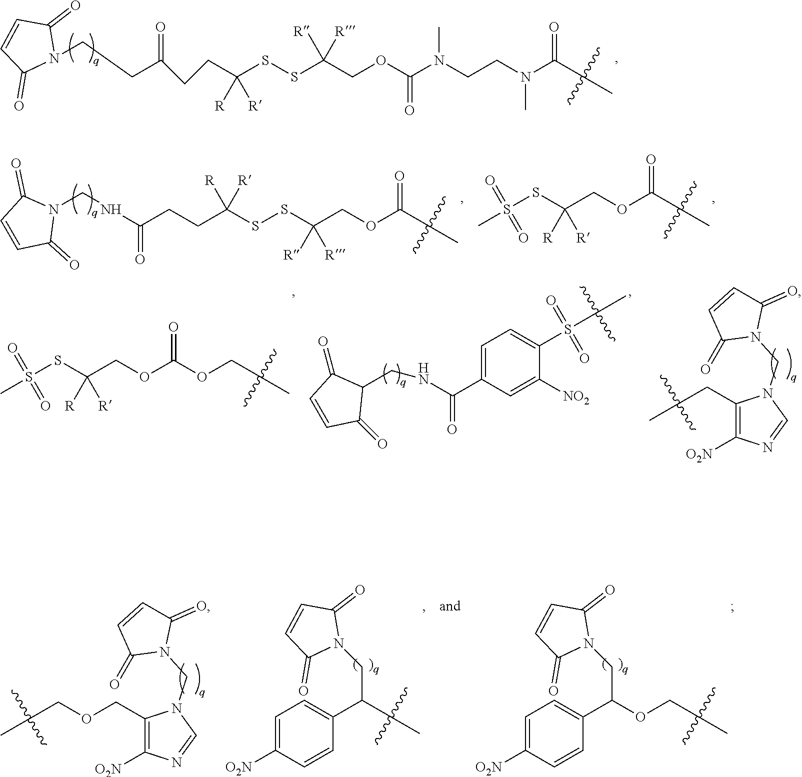
wherein:
q is an integer from 2 to 10;
R, R′, R″, and R′″ are each independently selected from hydrogen, C₁-C₆alkoxyC₁-C₆alkyl, (C₁-C₆)₂NC₁-C₆alkyl, and C₁-C₆alkyl; or, two geminal R groups, together with the carbon atom to which they are attached, can form a cyclobutyl or cyclopropyl ring and [symbol]
is the point of attachment to X.

61. The method of claim 52, wherein L′ is an acid cleavable linker precursor.

62. The method of claim 52 or 61, wherein L′ is selected from the group consisting of 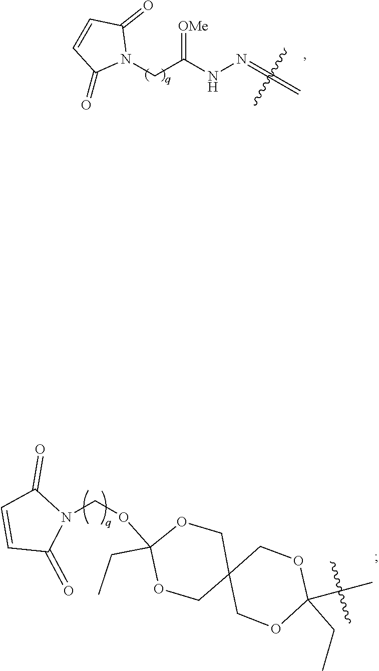
wherein:
q is an integer from 2 to 10; and [symbol]
is the point of attachment to X.

63. The method of claim 52, wherein L′ is a click-to-release linker precursor.

64. The method of claim 52 or 63, or a pharmaceutically acceptable salt thereof, wherein L′ is selected from 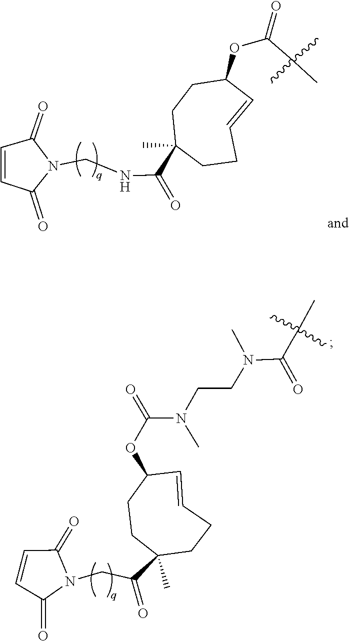
wherein:
q is an integer from 2 to 10; and [symbol]
is the point of attachment to X.

65. The method of claim 52, wherein L′ is a pyrophosphatase cleavable linker precursor.

66. The method of claim 65, wherein L′ is 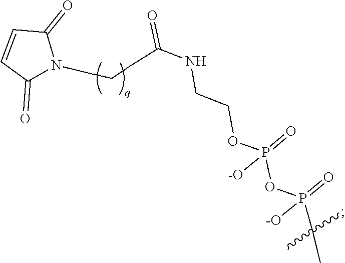
wherein:
q is an integer from 2 to 10; [symbol]
is the point of attachment to X.

67. The method of claim 52, wherein L′ is a beta-glucoronidase cleavable linker precursor.

68. The method of claim 52 or 67, wherein L′ is selected from 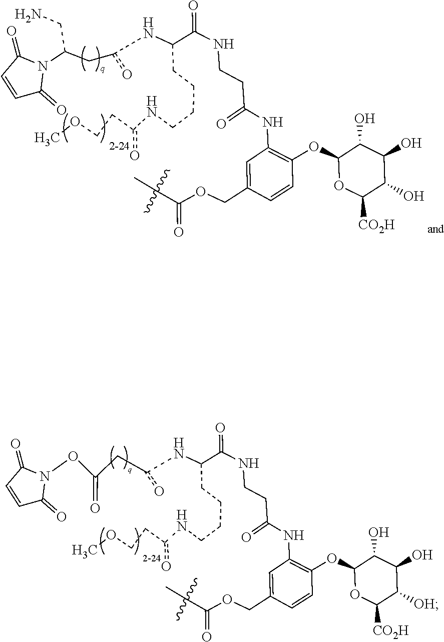
wherein:
q is an integer from 2 to 10;
---- is absent or a bond; and [symbol]
is the point of attachment to X.

69. The method of any one of claims 45 to 68, wherein the structure L′-V is reacted with a binding moiety, which comprises an antibody or an antigen binding portion thereof.

70. The method of claim 69, wherein the antibody or antigen binding portion thereof binds to a surface antigen.

71. The method of claim 70, wherein the surface antigen comprises 5T4, ACE, ADRB3, AKAP-4, ALK, Androgen receptor, AOC3, APP, Axin1, AXL, B7H3, B7-H4, BCL2, BCMA, bcr-abl, BORIS, BST2, C₂₄₂, C_{4.4}a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudine 3, CLDN6, CLEC12A, CLL-1, cll3, c-MET, Crypto 1 growth factor, CS1, CTLA-4, CXCR2, CXORF61, Cyclin B1, CYP1B1, Cadherin-3, Cadherin-6, DLL3, E7, EDNRB, EFNA4, EGFR, EGFRvIII, ELF2M, EMR2, ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GloboH, GM3, GPC1, GPC2, GPC3, gplOO, GPNMB, GPR20, GPRC5D, GUCY2C, HAVCR1, HER2, HER3, HGF, HMI.24, HMWMAA, HPV E6, hTERT, human telomerase reverse transcriptase, ICAM, ICOS-L, IFN-α, IFN-γ, IGF-I receptor, IGLL1, IL-2 receptor, IL-4 receptor, IL-13Ra2, IL-l lRa, IL-1, IL-12, IL-23, IL-13, IL-22, IL-4, IL-5, IL-6, interferon receptor, integrins (including α₄, αᵥβ₃, αᵥβ₅, αᵥβ₆, α₁β₄, α₄β₁, α₄β₇, α₅β₁, α₆β₄, α_{IIb}β₃ integrins), Integrin alphaV, intestinal carboxyl esterase, KIT, LAGE-la, LAIR1, LAMP-1, LCK, Legumain, LewisY, LFA-1(CD11a), L-selectin(CD62L), LILRA2, LIV-1, LMP2, LRRC15, LY6E, LY6K, LY75, MAD-CT-1, MAD-CT-2, MAGE Al, MelanA/MARTl, Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2, OY-TES1, p53, p53 mutant, PANX3, PAP, PAX3, PAX5, p-CAD, PCTA-1/Galectin 8, PD-L1, PD-L2, PDGFR, PDGFR-beta, phosphatidylserine, PIK3CA, PLAC1, Polysialic acid, Prostase, prostatic carcinoma cell, prostein, Pseudomonas aeruginosa, rabies, survivin and telomerase, PRSS21, PSCA, PSMA, PTK7, RAGE-1, RANKL, Ras mutant, respiratory syncytial virus, Rhesus factor, RhoC, RON, ROR1, ROR2, RU1, RU2, sarcoma translocation breakpoints, SART3, SLAMF7, SLC44A4, sLe, SLITRK6, sperm protein 17, sphingosine-I-phosphate, SSEA-4, SSX2, STEAP1, TAG72, TARP, TCRβ, TEM1/CD248, TEM7R, tenascin C, TF, TGF-1, TGF-β2, TNF-α, TGS5, Tie 2, TIM-1, Tn Ag, TRAC, TRAIL-R1, TRAIL-R2, TROP-2, TRP-2, TRPV1, TSHR, tumor antigen CTAA16.88, tyrosinase, UPK2, VEGF, VEGFR1, VEGFR2, vimentin, WTl, XAGE1, or combinations thereof.

72. The method of claim 70, wherein the surface antigen comprises HER2, CD20, CD38, CD33, BCMA, CD138, EGFR, FGFR4, GD2, PDGFR, TEM1/CD248, TROP-2, or combinations thereof.

73. The method of claim 69, wherein the antibody comprises rituximab, trastuzumab, gemtuzumab, pertuzumab, obinutuzumab, ofatumumab, olaratumab, ontuximab, isatuximab, Sacituzumab, U3-1784, daratumumab, STI-6129, lintuzumab, huMy9-6, balantamab, indatuximab, cetuximab, dinutuximab, anti-CD38 A2 antibody, huAT13/5 antibody, alemtuzumab, ibritumomab, tositumomab, bevacizumab, panitumumab, tremelimumab, ticilimumab, catumaxomab, oregovomab, or veltuzumab.

74. The method of claim 73, wherein the antibody is rituximab, trastuzumab, pertuzumab, huMy9-6, lintuzumab, or gemtuzumab.

75. The method of any one of claims 45 to 74, wherein V has Formula (a): 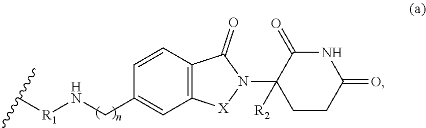
or a pharmaceutically acceptable salt, solvate, or stereoisomer thereof, wherein:
n is 0 or 1;
X is CH₂, C═O, or C═S;
R₁ is:
a) —(CH₂)ₘR₃ or —CO(CH₂)ₘR₃, wherein
m is 0, 1, 2, or 3; and
R₃ is 5-10 membered aryl or heteroaryl, optionally substituted with one or more halogen;
b) —C═YR₄, wherein
Y is O or S; and
R₄ is:
(C₀-C₁₀)alkyl-(5 to 10 membered heteroaryl or heterocycle), said heteroaryl or heterocycle optionally substituted with one or more of (C₁-C₆)alkyl, halogen, oxo, (C₁-C₆)alkoxy, or —Z—(C₁-C₆)alkyl, wherein Z is S or SO₂, and wherein said (C₁-C₆)alkyl may be optionally substituted with one or more halogen;
(C₀-C₁₀)alkyl-(5 to 10 membered aryl), said aryl optionally substituted with one or more of: halogen; (C₁-C₆)alkoxy, itself optionally substituted with one or more halogen; (C₁-C₆)alkyl, itself optionally substituted with one or more halogen; or —Z—(C₁-C₆)alkyl, wherein Z is S or SO₂, and wherein said (C₁-C₆)alkyl may be optionally substituted with one or more halogen; or (C₁-C₆)alkyl-CO—O—R₁₂, wherein R₁₂ is H or (C₁-C₆)alkyl; or
c) —C═ZNHR₆, wherein
Z is O or S; and
R₆ is:
5 to 10 membered aryl or heteroaryl, optionally substituted with one or more of: halogen; cyano; (C₁-C₆)alkylenedioxy; (C₁-C₅)alkoxy, itself optionally substituted with one or more halogen; (C₁-C₆)alkyl, itself optionally substituted with one or more halogen; or (C₁-C₆)alkylthio, itself optionally substituted with one or more halogen; and
R₂ is H or (C₁-C₆)alkyl.

76. The method of claim 75, wherein:
n is 0 or 1;
X is CH₂ or C═0;
R₁ is —(CH₂)ₘR₃, wherein
m is 0, 1, 2, or 3; and
R₃ is 5-10 membered aryl or heteroaryl, optionally substituted with one or more halogen.

77. The method of claim 75, wherein:
n is 1;
X is CH₂ or C═O;
R₁ is:
C═OR₄ or —C═SR₄ wherein
R₄ is:
(C₀-C₁₀)alkyl-(5 to 10 membered heteroaryl or heterocycle), said heteroaryl or heterocycle optionally substituted with one or more of (C₁-C₆)alkyl, halogen, oxo, (C₁-C₆)alkoxy, or —Z—(C₁-C₆)alkyl, wherein Z is S or SO₂, and wherein said (C₁-C₆)alkyl may be optionally substituted with one or more halogen;
(C₀-C₁₀)alkyl-(5 to 10 membered aryl), said aryl optionally substituted with one or more of: halogen; (C₁-C₆)alkoxy, itself optionally substituted with one or more halogen; (C₁-C₆)alkyl, itself optionally substituted with one or more halogen; or —Z—(C₁-C₆)alkyl, wherein Z is S or SO₂, and wherein said (C₁-C₆)alkyl may be optionally substituted with one or more halogen; or (C₁-C₆)alkyl-CO—O—R₁₂, wherein R₁₂ is H or (C₁-C₆)alkyl.

78. The method of claim 75, wherein:
n is 1;
X is CH₂ or C═0;
R₁ is:
C═ONHR₆ or —C═SNHR₆, wherein
R₆ is:
5 to 10 membered aryl or heteroaryl, optionally substituted with one or more of: halogen; cyano; (C₁-C₆)alkylenedioxy; (C₁-C₅)alkoxy, itself optionally substituted with one or more halogen; (C₁-C₆)alkyl, itself optionally substituted with one or more halogen; or (C₁-C₆)alkylthio, itself optionally substituted with one or more halogen.

79. The method of any one of claims 45 to 74, wherein V has Formula (b): 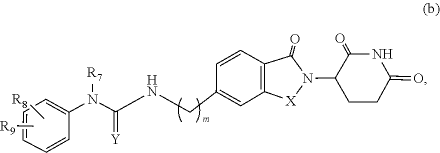
or a pharmaceutically acceptable salt, Solvate, prodrug, or stereoisomer thereof, wherein:
X is C(═O) or CH₂;
Y is O, cyanamido (N—≡N), or amido (NH);
m is an integer of 0, 1, 2, or 3;
R₇ is hydrogen or C₁₋₆ alkyl;
R₈ is hydrogen, —NO₂, C₁₋₁₀ alkyl, C₀₋₆ alkyl-(5 to 10 membered heteroaryl), C₀₋₆ alkyl-(5 to 6 membered heterocyclyl), C₀₋₆ alkyl-OH, C₀₋₄ alkyl-NH₂, —NHCO—C₁₋₆ alkyl, —OR₂₁, or —(CH₂—Z)₀₋₂-(5 to 10 membered heteroaryl), where each heteroaryl and heterocyclyl is optionally substituted with one or more C₁₋₆ alkyl;
R₉ is hydrogen, halogen, —NO₂, C₀₋₆ alkyl-(5 to 10 membered heteroaryl), C₀₋₆ alkyl-(5 to 6 membered heterocyclyl), C₀₋₆ alkyl-OH, C₀₋₄ alkyl-NH₂, —NHCO—C₁₋₆ alkyl, —OR₂₁, or —(CH₂—Z)₀₋₂-(5 to 10 membered heteroaryl), where each heteroaryl and heterocyclyl is optionally substituted with one or more C₁₋₆ alkyl;
R₂₁ is C₆₋₁₀ aryl, 5 to 10 membered heteroaryl, 5 to 6 membered heterocyclyl, or —CO(CH₂)₀₋₂R₂₂, wherein the aryl, heteroaryl, and heterocyclyl are each optionally substituted with one or more C₁₋₆ alkyl;
R₂₂ is —NH₂ or 5 to 6 membered heterocyclyl; and
Z is CH₂, NH, or O.

80. The method of any one of claims 45 to 74, wherein V has Formula (c): 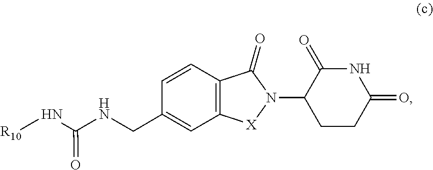
or a pharmaceutically acceptable salt, Solvate, prodrug, or stereoisomer thereof, wherein:
X is C(═O) or CH₂;
m is an integer of 0, 1, 2, or 3;
R₁₀ is C₃₋₁₀ cycloalkyl, 5 to 10 membered heterocyclyl, 5 to 10 membered heteroaryl,
or C₀₋₄ alkyl-NR₄₁R₄₂; wherein the cycloalkyl, heterocyclyl, and heteroaryl are each optionally
substituted with one or more halogen, C₁₋₆ alkyl, —CO—NR₄₃R₄₄, —COOR₄₅, or C₀₋₄ alkyl-C₆₋₁₀ aryl,
wherein the aryl itself may be optionally substituted with one or more halogen; and
R₄₁, R₄₂, R₄₃, R₄₄, and R₄₅ are each independently hydrogen or C₁₋₆ alkyl.

81. The method of any one of claims 45 to 74, wherein V has Formula (d) 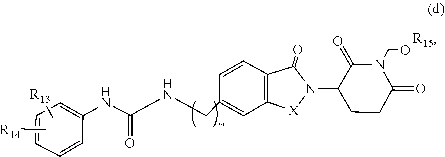
or a pharmaceutically acceptable salt, Solvate, prodrug, or stereoisomer thereof, wherein:
X is C(═O) or CH₂;
m is an integer of 0, 1, 2, or 3;
R₁₃ and R₁₄ are each independently: hydrogen, halo, C₁₋₆ alkyl, oxo, —NO₂, —Z—C₀₋₆ alkyl-(5 to 10 membered heteroaryl), C₀₋₆ alkyl-(5 to 6 membered heterocyclyl), C₀₋₆ alkyl-OH, C₀₋₄ alkyl-NH₂, —NHCO—C₁₋₆ alkyl, —OR₂₁, or —(CH₂—Z)₀₋₂-(5 to 10 membered heteroaryl),
wherein Z is S or SO₂;
wherein R₂₁ is defined above;
wherein each heteroaryl and heterocyclyl above is optionally substituted with one or more C₁₋₆ alkyl; and
wherein the alkyl or alkoxy above may be optionally substituted with one or more: halogen; cyano; nitro; amino: C₁₋₆ alkylidenedioxy; C₁₋₆ alkoxy, itself optionally substituted with one or more halogens; or C₁₋₆ alkylthio, itself optionally substituted with one or more halogens:
R₁₅ is COR₇₁ or PO(OR₇₂)(OR₇₃);
R₇₁ is C₁₋₁₀ alkyl, C₆₋₁₀ aryl, or 5 to 6 membered heterocyclyl; wherein the alkyl, aryl, heterocyclyl may be optionally substituted with one or more amino, C₁₋₆ alkylamino, di(C₁₋₆ alkyl)amino, or —COOR₇₄; and
R₇₂, R₇₃, and R₇₄ are each independently hydrogen or C₁₋₁₀ alkyl.

82. The method of any one of claims 45 to 74, wherein V has Formula (e) 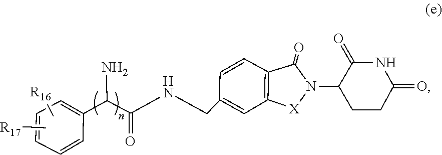
or a pharmaceutically acceptable salt, Solvate, prodrug, or stereoisomer thereof, wherein:
X is C(═O) or CH:
n is an integer of 0 or 1;
R is hydrogen or halo; and
R is hydrogen, amino, or 5 to 10 membered heteroaryl or heterocyclyl.

83. The method of any one of claims 45 to 74, wherein V has Formula (f) 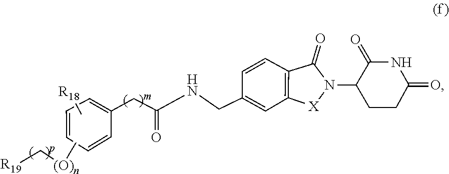
or a pharmaceutically acceptable salt, Solvate, prodrug, or stereoisomer thereof, wherein:
X is CH or C═O;
m and n are each independently 0 or 1;
p is 0, 1, 2, or 3:
R₁₉ is 5 to 6 membered heterocyclyl, optionally substituted with C₁₋₆ alkyl; and
R₁₈ is hydrogen or halogen.

84. The method of any one of claims 45 to 74, wherein V has Formula (g) 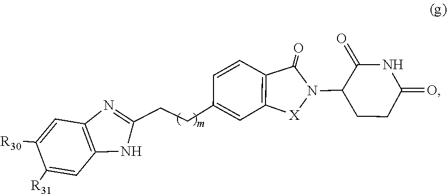
or a pharmaceutically acceptable salt, Solvate, prodrug, or stereoisomer thereof, wherein:
X is CH or C═O;
m is an integer of 0, 1, 2, or 3:
R₃₀ and R₃₁ are each independently hydrogen, halo, C₁₋₆ alkyl, or C₁₋₁₀aryloxy, wherein the alkyl and aryl are each optionally substituted with one or more halo.

85. The method of claim 79, or a pharmaceutically acceptable salt, solvate, or stereoisomer thereof, wherein when R₇ is hydrogen, then R₈ is not hydrogen or C_{1_6} alkyl; when Y is O, then R₉ is not halogen; and when Y is O and R₉ is halogen, then R₈ is C_{0_6} alkyl-(5-6membered heterocyclyl).

86. The method of claim 82, or a pharmaceutically acceptable salt, solvate, or stereoisomer thereof, wherein when m is 0, R₁₇ is not hydrogen.

87. The conjugate of claim 33, or a pharmaceutically acceptable salt, solvate, or stereoisomer thereof, wherein:
n is 1;
X is CH₂ or C═0;
R₁ is:
C═OR₄, wherein
R₄ is:
(C₀-C₁₀)alkyl-(5 to 10 membered heteroaryl or heterocycle), said heteroaryl or heterocycle optionally substituted with one or more of (C₁-C₆)alkyl, halogen, oxo, (C₁-C₆)alkoxy, or —Z—(C₁-C₆)alkyl, wherein Z is S or SO₂, and wherein said (C₁-C₆)alkyl may be optionally substituted with one or more halogen;
(C₀-C₁₀)alkyl-(5 to 10 membered aryl), said aryl optionally substituted with one or more of: halogen; (C₁-C₆)alkoxy, itself optionally substituted with one or more halogen; (C₁-C₆)alkyl, itself optionally substituted with one or more halogen; or —Z—(C₁-C₆)alkyl, wherein Z is S or SO₂, and wherein said (C₁-C₆)alkyl may be optionally substituted with one or more halogen; or (C₁-C₆)alkyl-CO—O—R₁₂, wherein R₁₂ is H or (C₁-C₆)alkyl.

88. The conjugate of claim 87, or a pharmaceutically acceptable salt, solvate, or stereoisomer thereof, wherein Formula (a) is

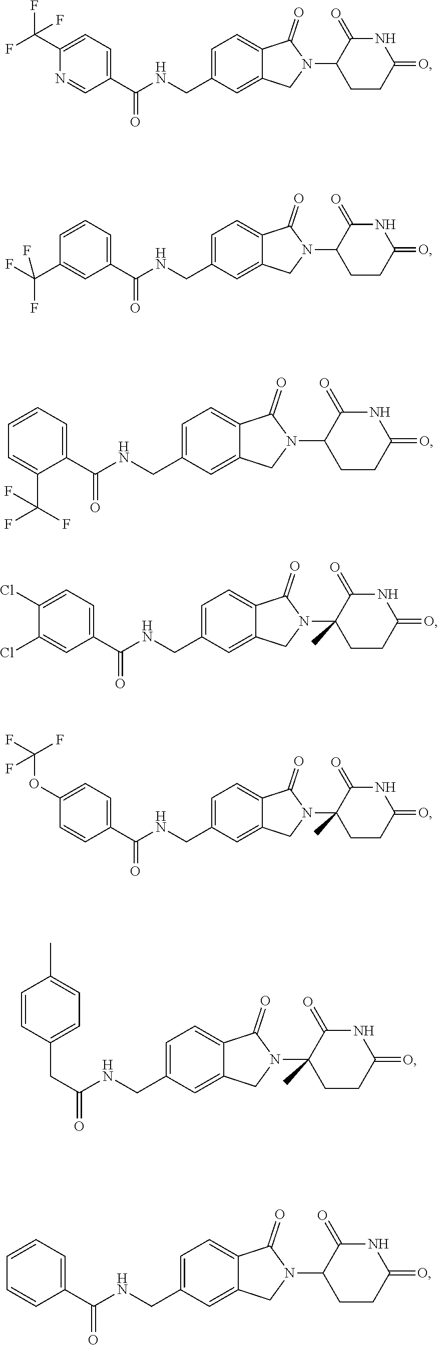

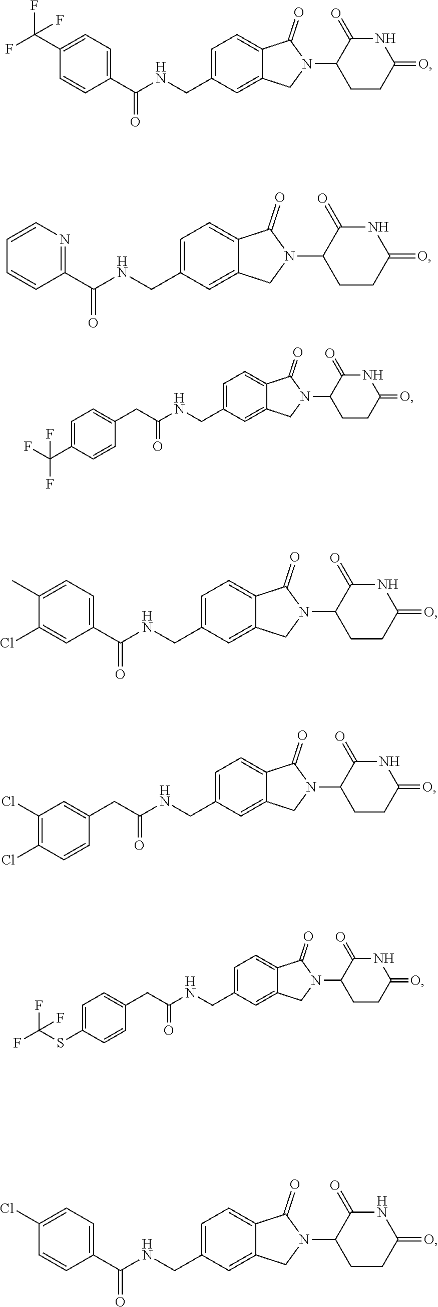

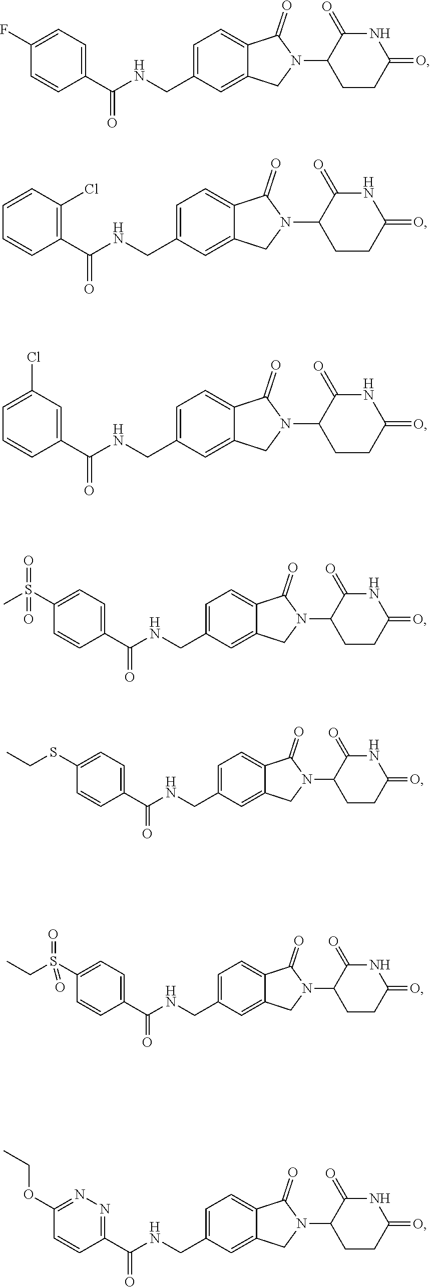

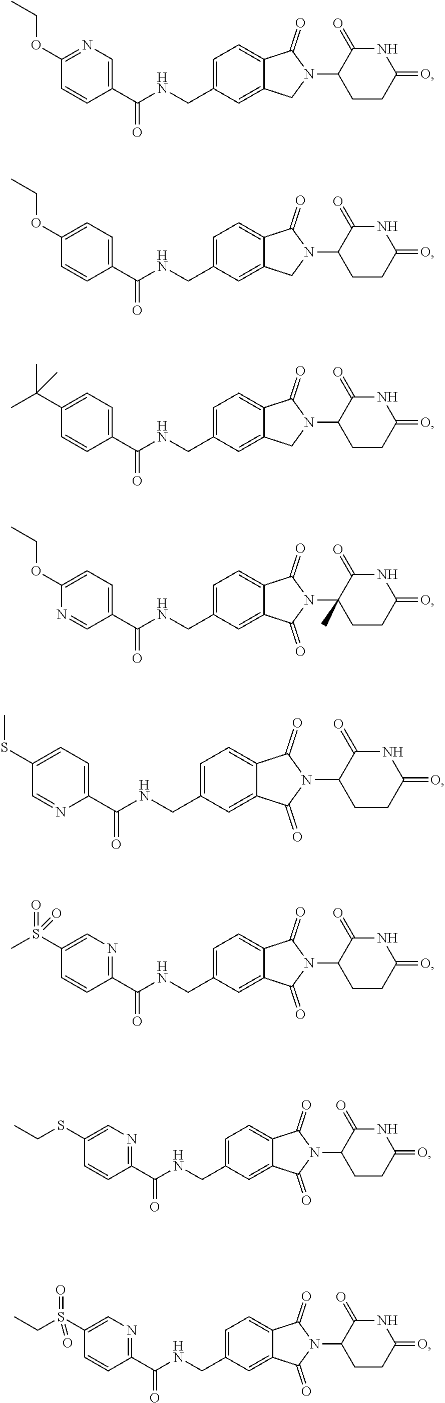

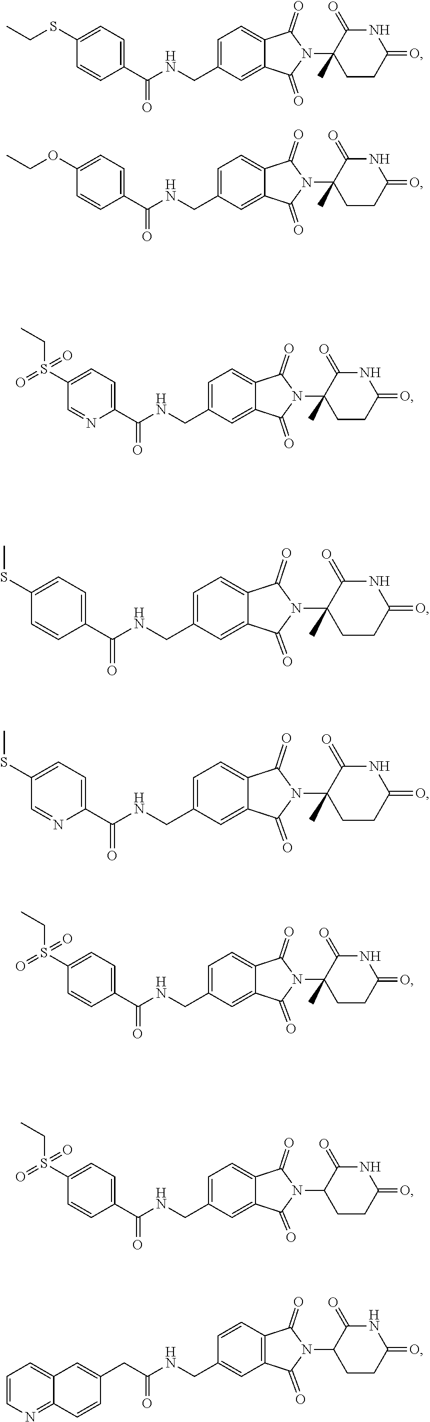

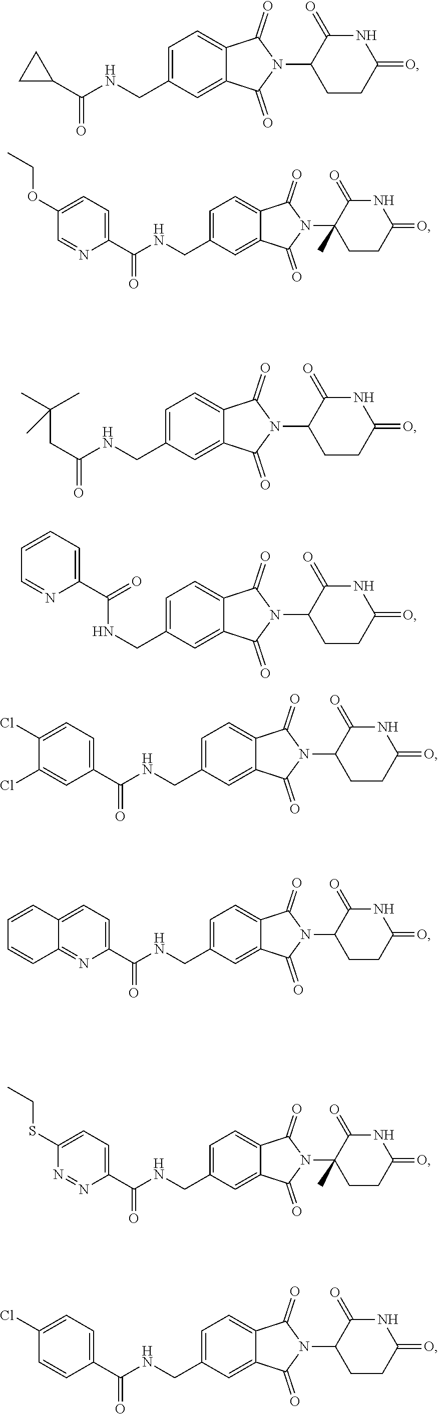

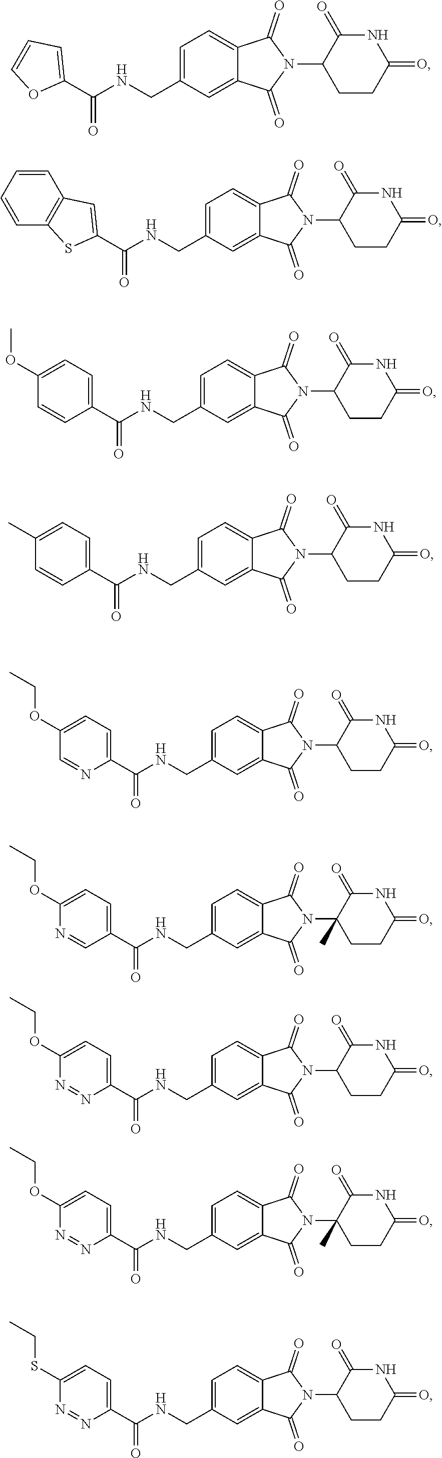

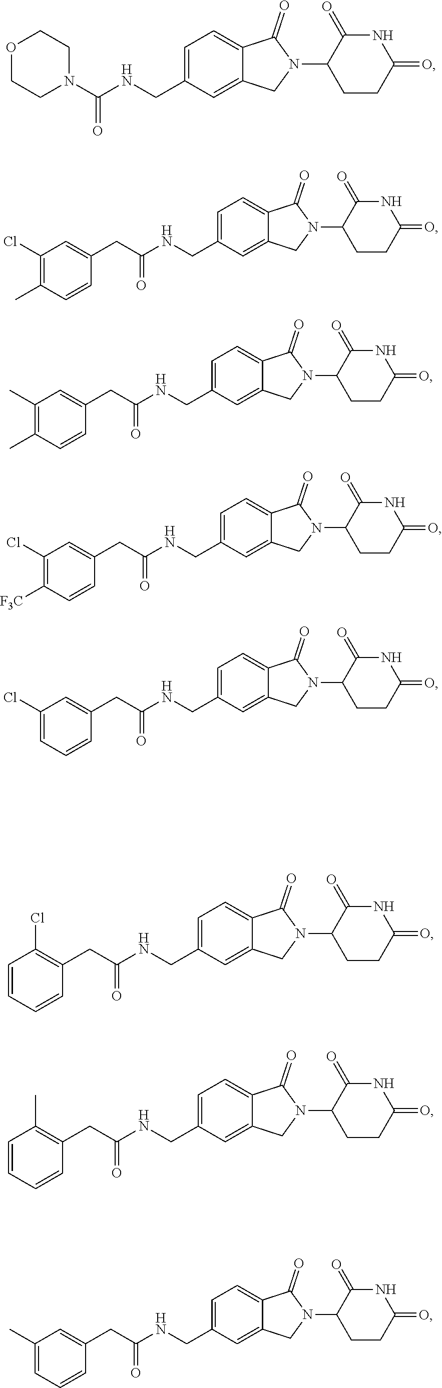

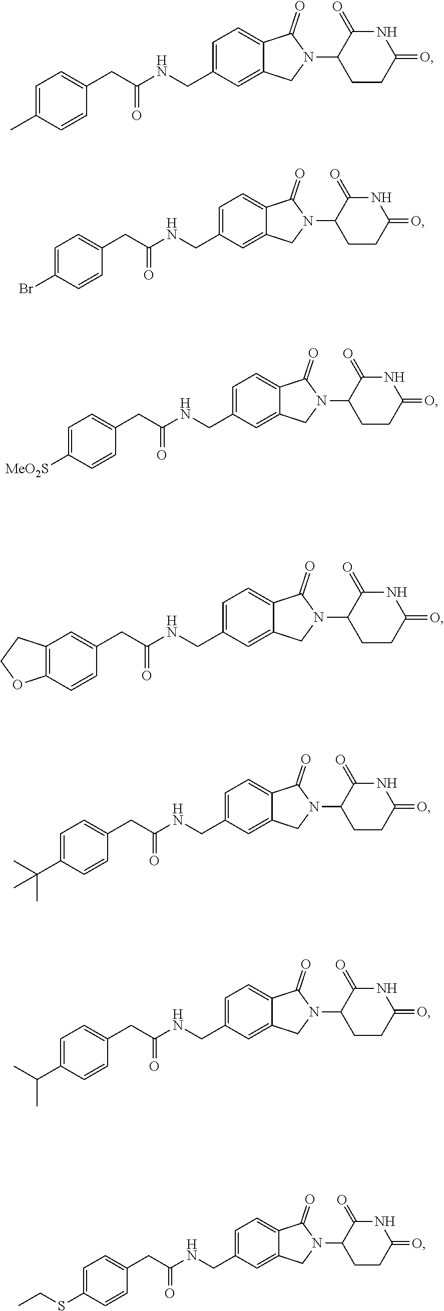

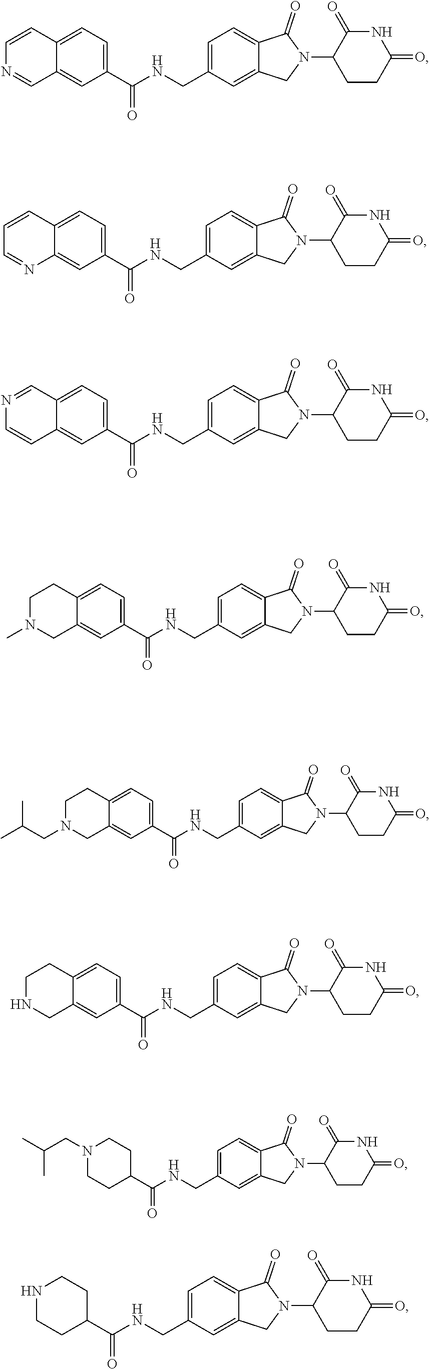

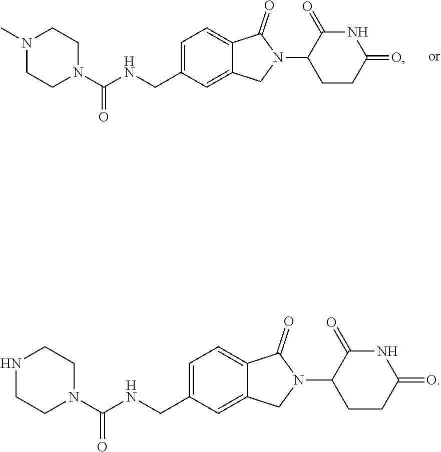 [SMILES: CN1CCN(C(=O)NCc2ccc3c(c2)CN(C2CCC(=O)NC2=O)C3=O)CC1 ; "or" ; O=C1CCC(N2Cc3cc(CNC(=O)N4CCNCC4)ccc3C2=O)C(=O)N1]

89. The conjugate of claim 36, or a pharmaceutically acceptable salt, solvate, or stereoisomer thereof, wherein Formula (a) is

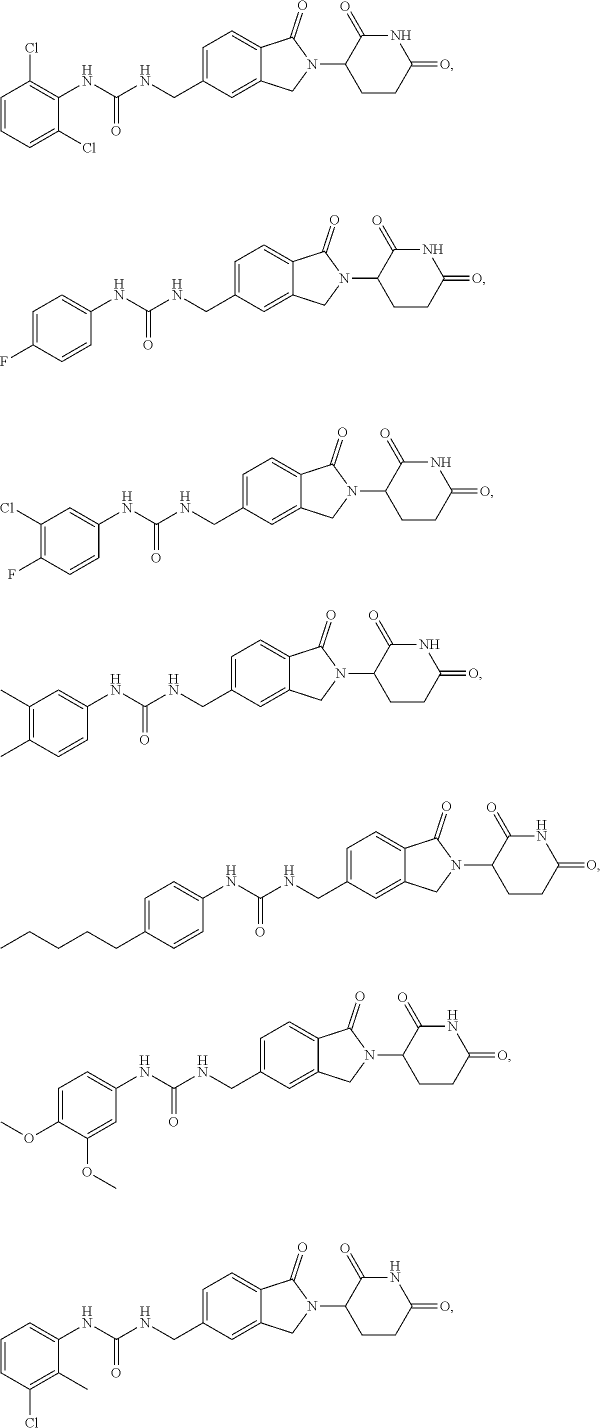

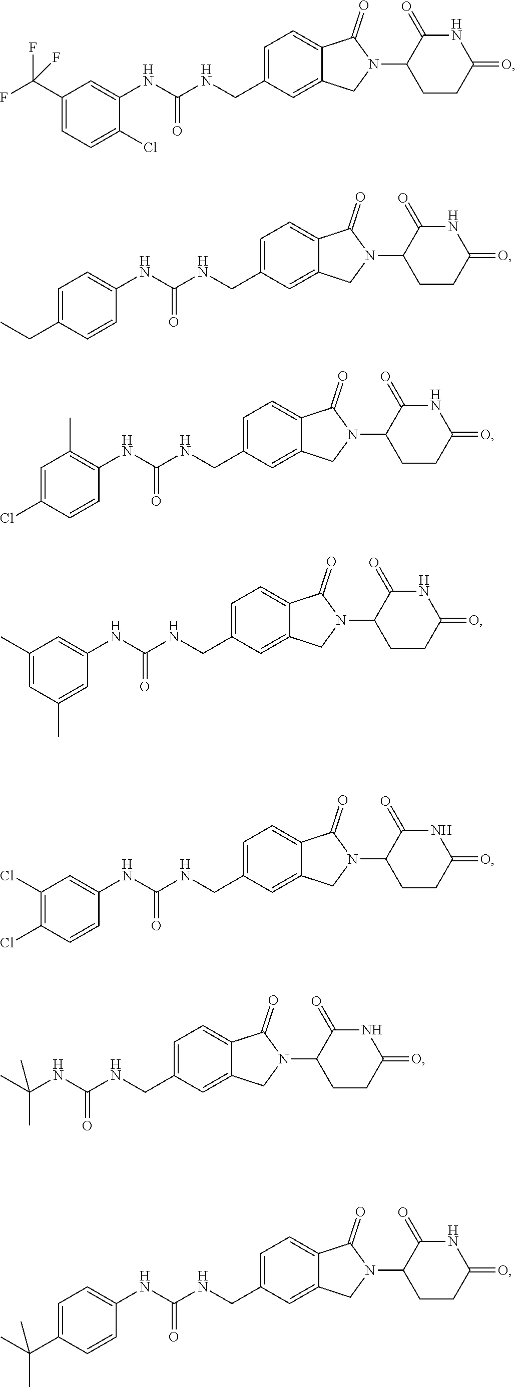

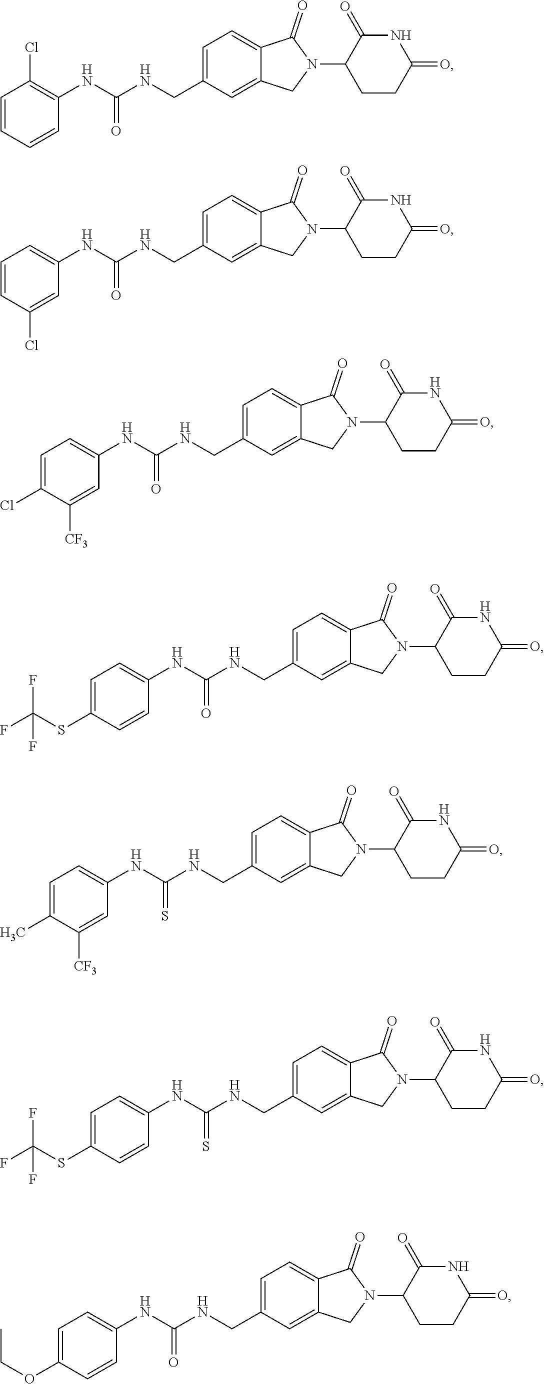

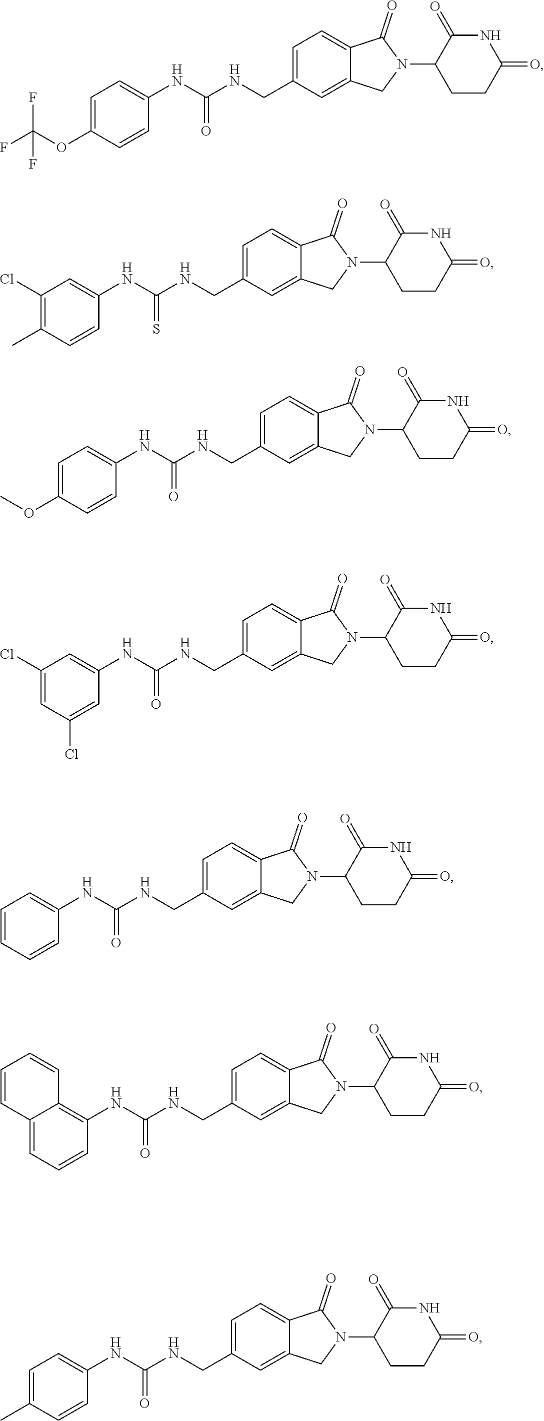

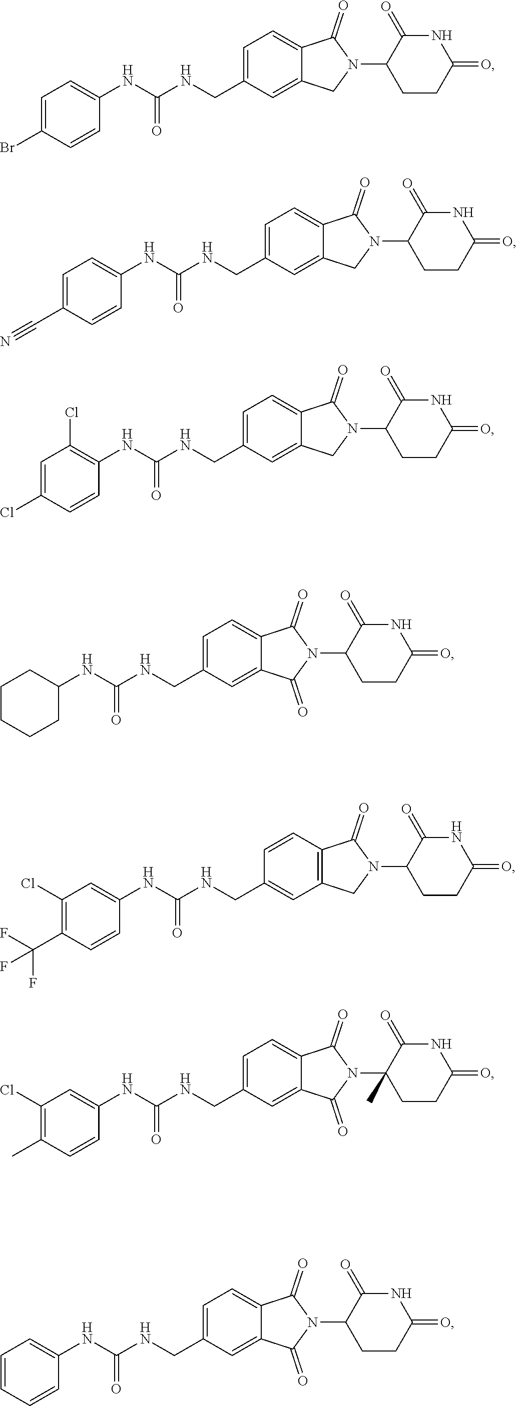

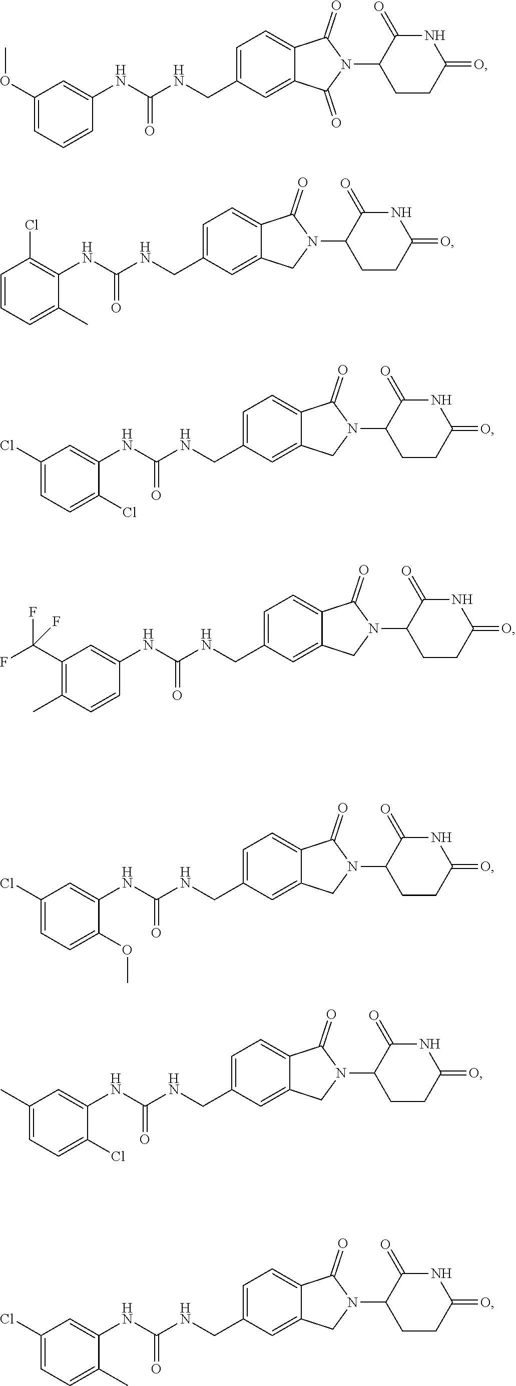

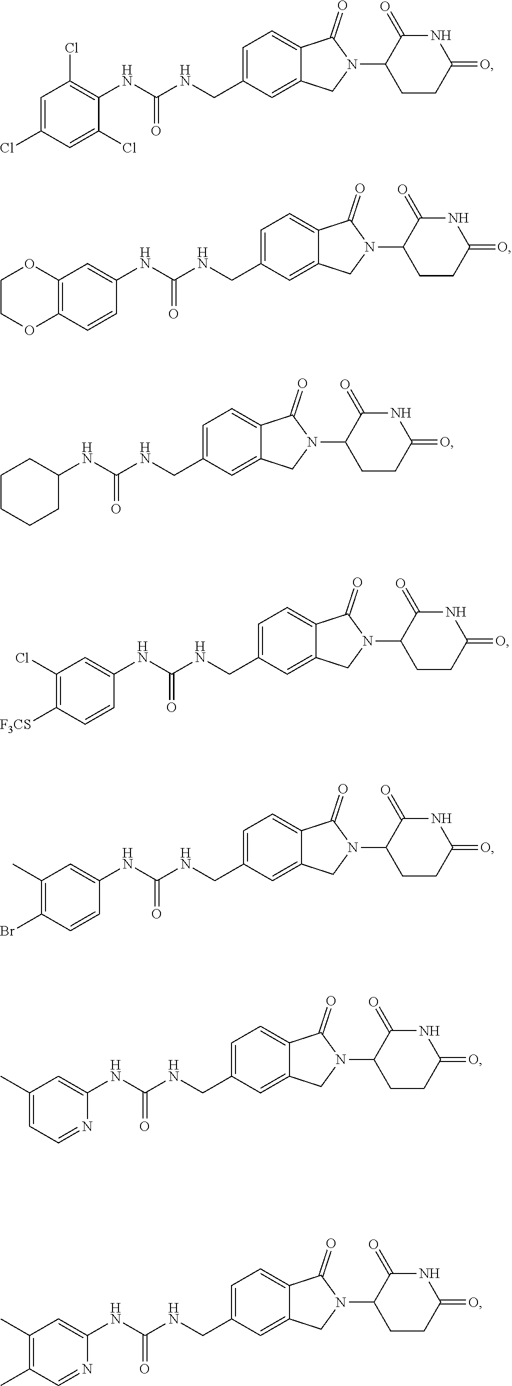

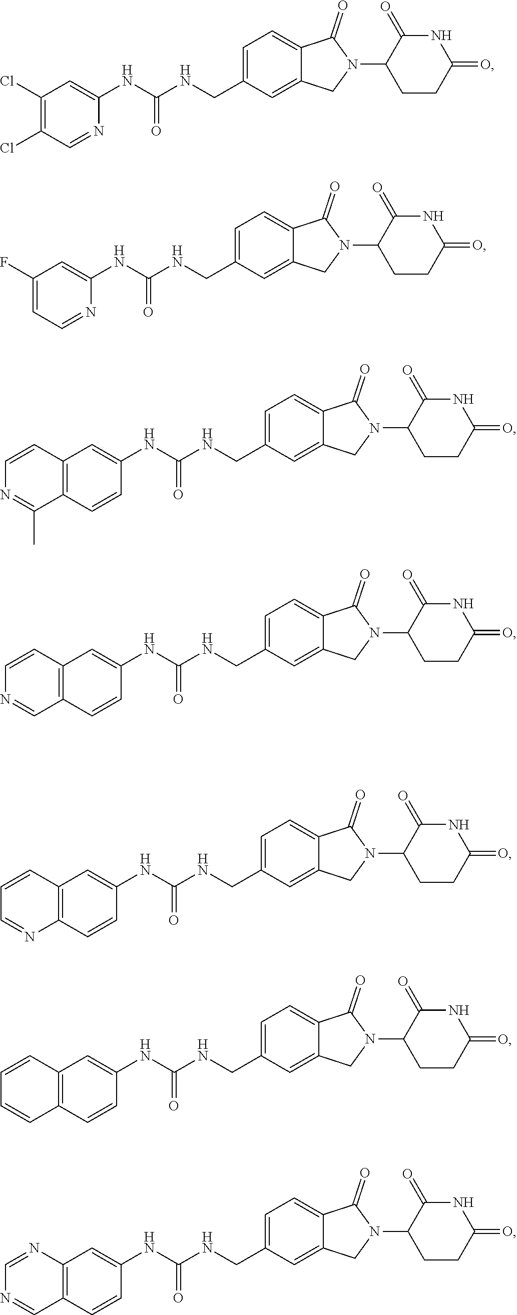

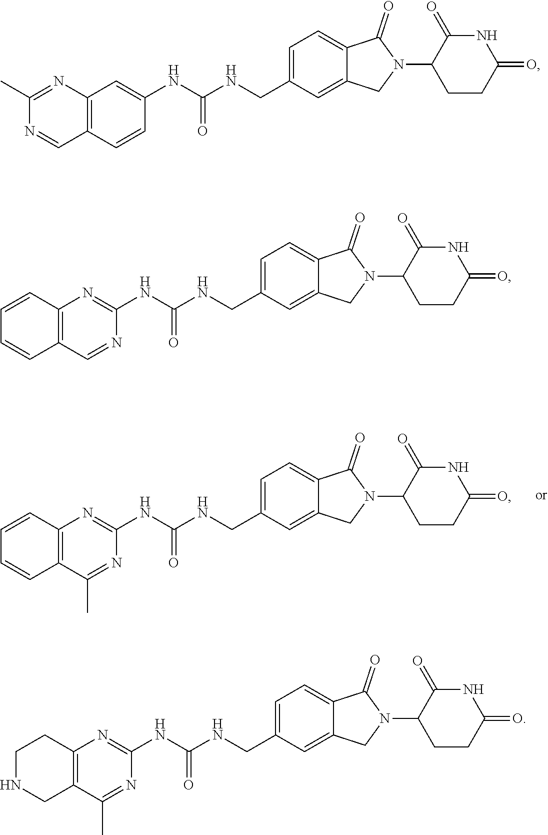 [SMILES: Cc1ncc2ccc(NC(=O)NCc3ccc4c(c3)CN(C3CCC(=O)NC3=O)C4=O)cc2n1 ; O=C1CCC(N2Cc3cc(CNC(=O)Nc4ncc5ccccc5n4)ccc3C2=O)C(=O)N1 ; Cc1nc(NC(=O)NCc2ccc3c(c2)CN(C2CCC(=O)NC2=O)C3=O)nc2ccccc12 ; "or" ; Cc1nc(NC(=O)NCc2ccc3c(c2)CN(C2CCC(=O)NC2=O)C3=O)nc2c1CNCC2]

90. The method of claim 75, wherein:
n is 1;
X is CH₂ or C═0;
R₁ is:
C═OR₄, wherein
R₄ is:
(C₀-C₁₀)alkyl-(5 to 10 membered heteroaryl or heterocycle), said heteroaryl or heterocycle optionally substituted with one or more of (C₁-C₆)alkyl, halogen, oxo, (C₁-C₆)alkoxy, or —Z—(C₁-C₆)alkyl, wherein Z is S or SO₂, and wherein said (C₁-C₆)alkyl may be optionally substituted with one or more halogen;
(C₀-C₁₀)alkyl-(5 to 10 membered aryl), said aryl optionally substituted with one or more of: halogen; (C₁-C₆)alkoxy, itself optionally substituted with one or more halogen; (C₁-C₆)alkyl, itself optionally substituted with one or more halogen; or —Z—(C₁-C₆)alkyl, wherein Z is S or SO₂, and wherein said (C₁-C₆)alkyl may be optionally substituted with one or more halogen; or (C₁-C₆)alkyl-CO—O—R₁₂, wherein R₁₂ is H or (C₁-C₆)alkyl.

91. The method of claim 90, or a pharmaceutically acceptable salt, solvate, or stereoisomer thereof, wherein Formula (a) is

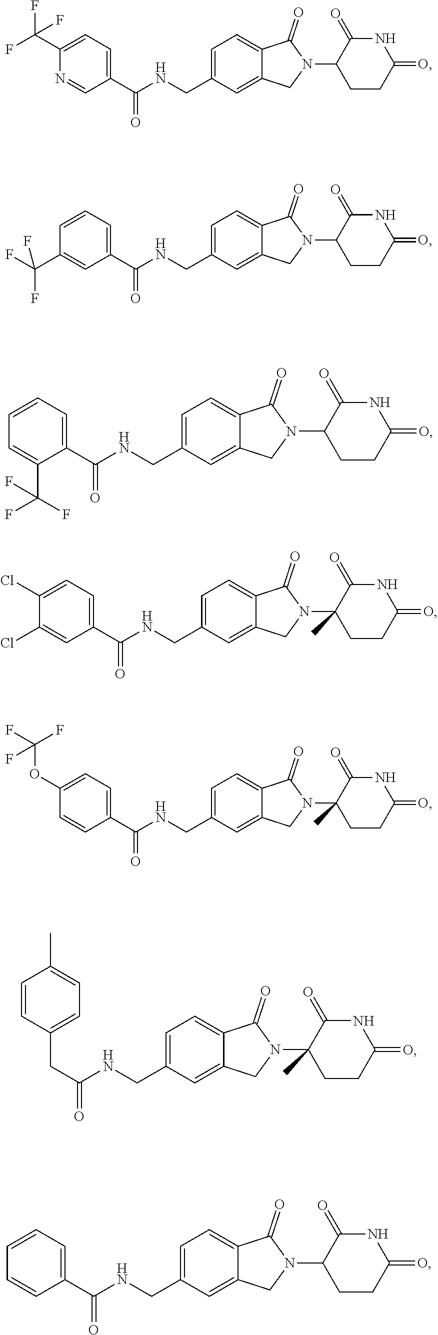

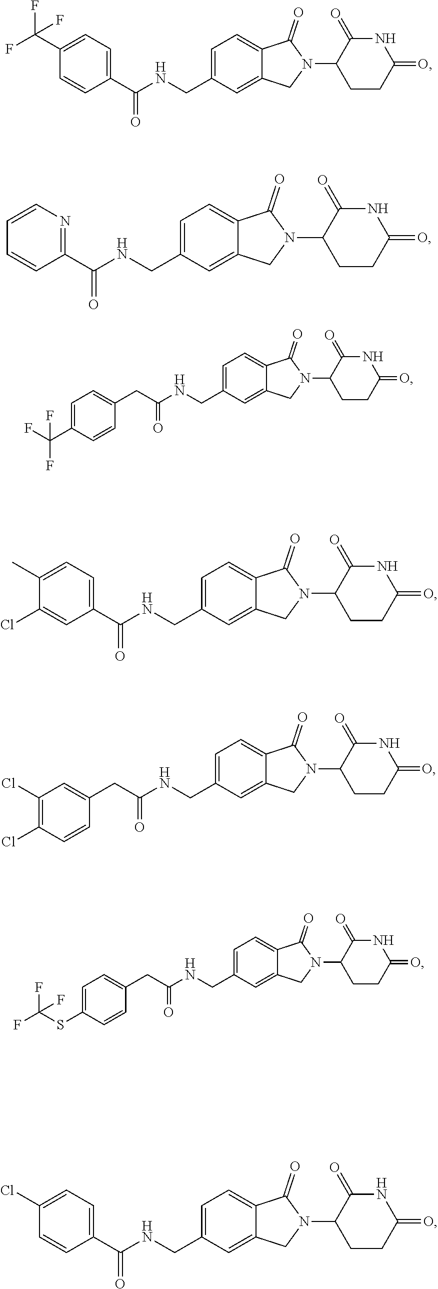

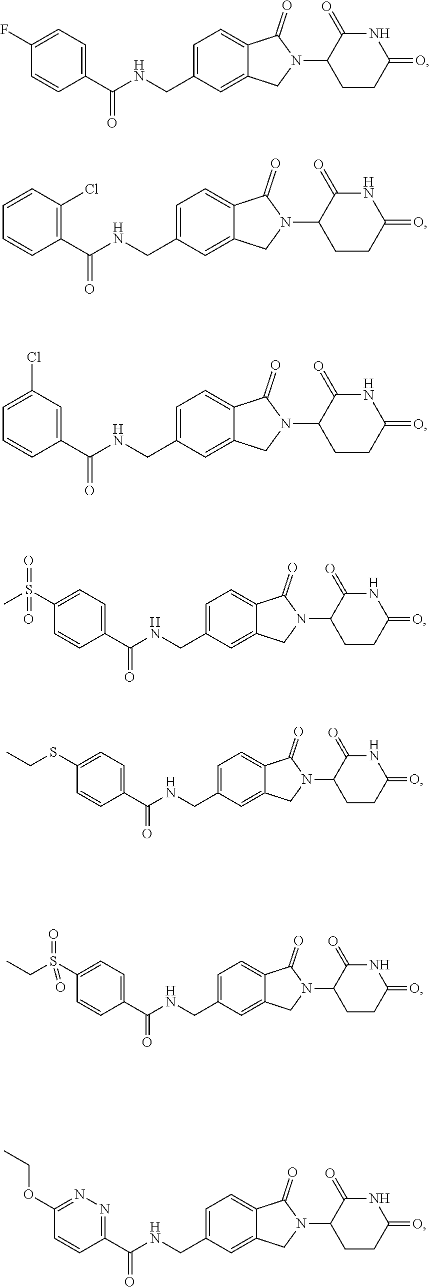

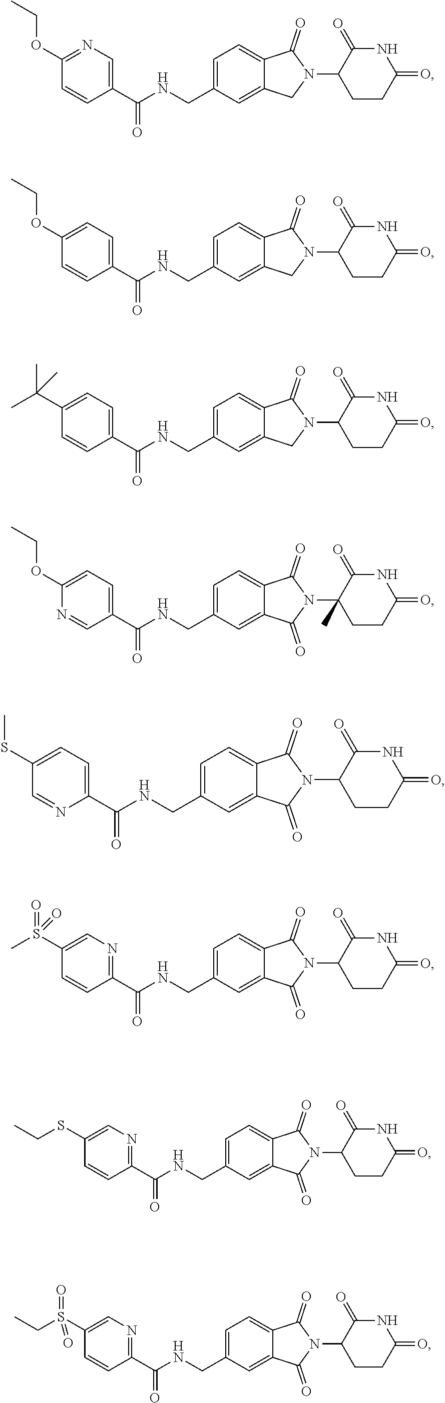

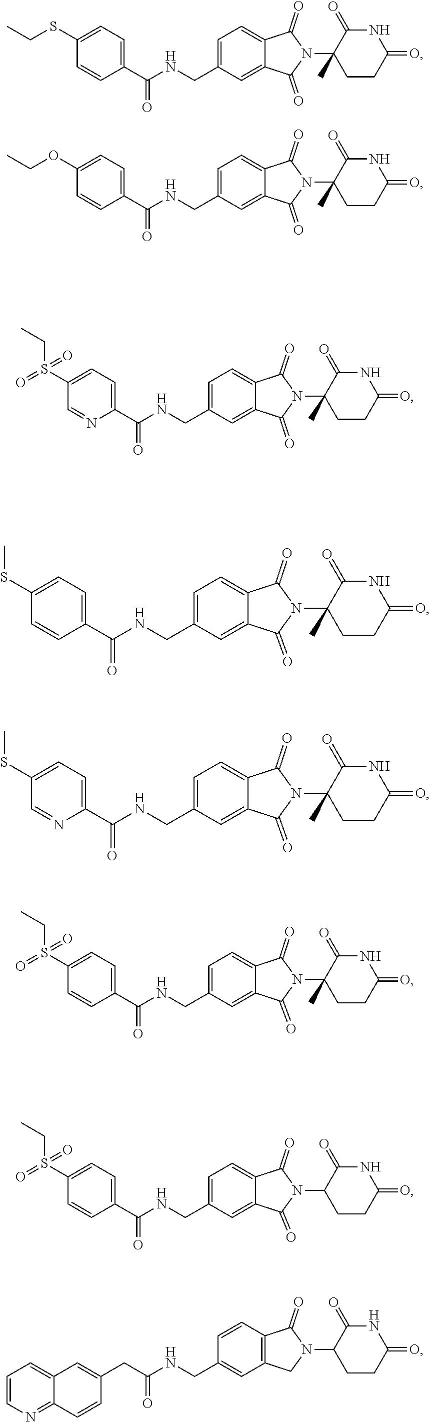

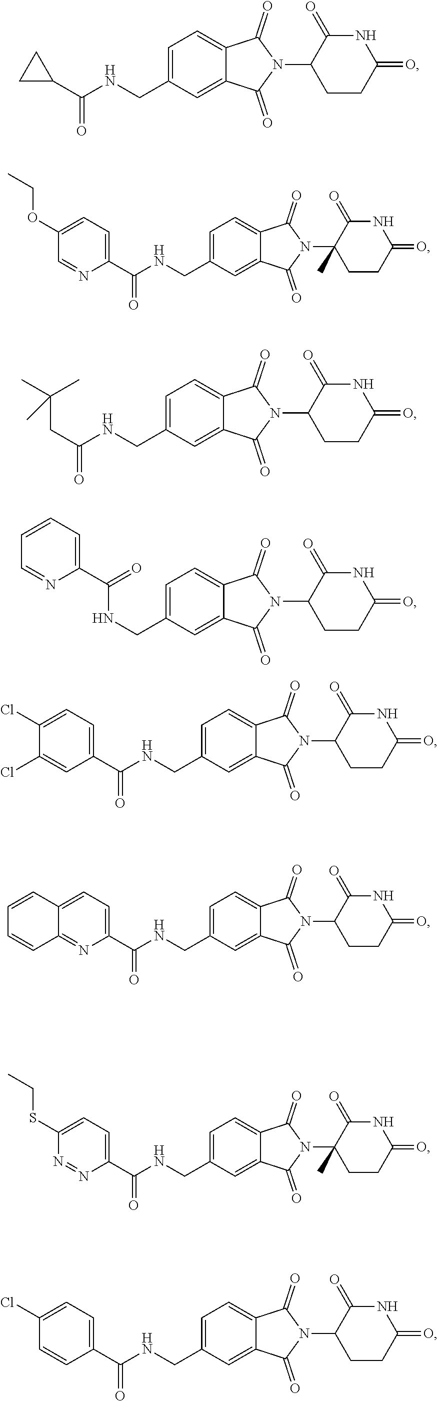

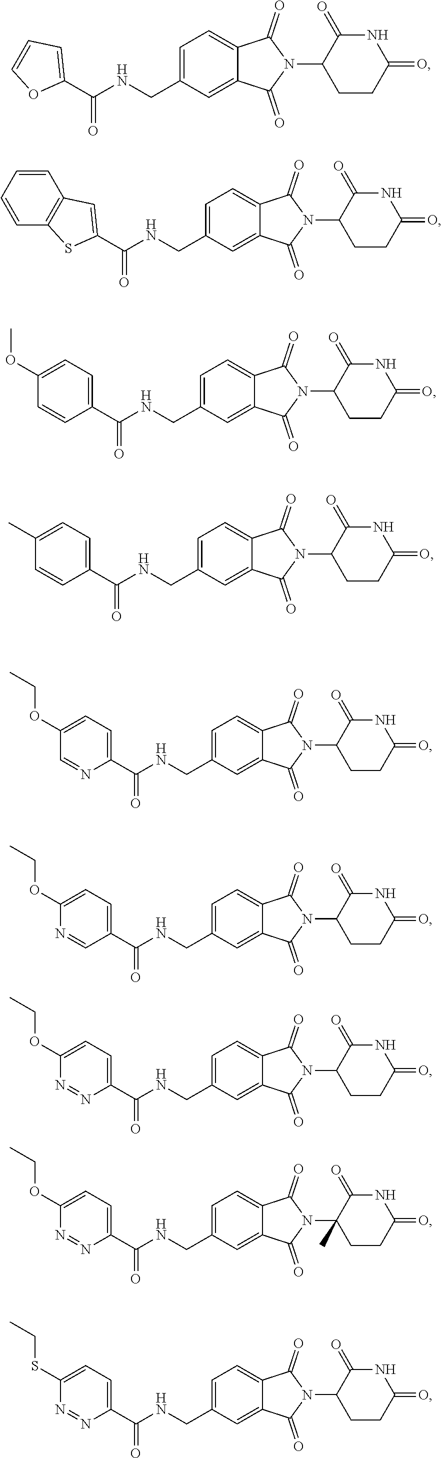

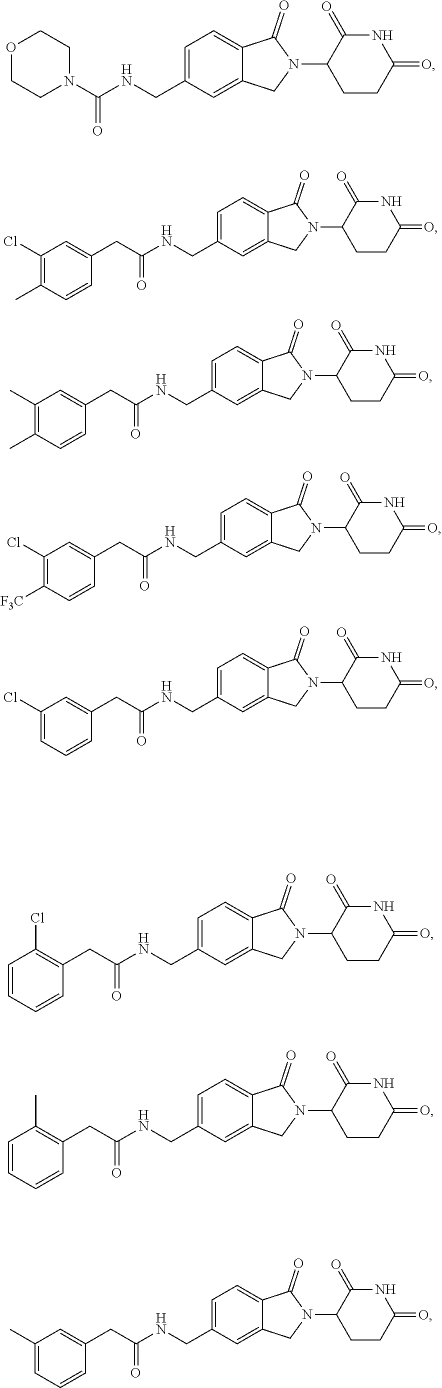

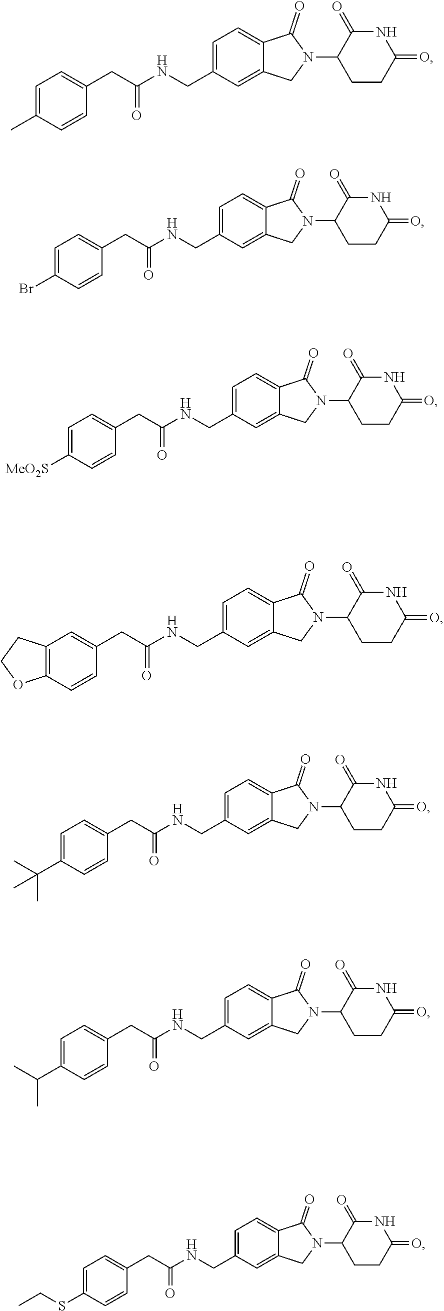

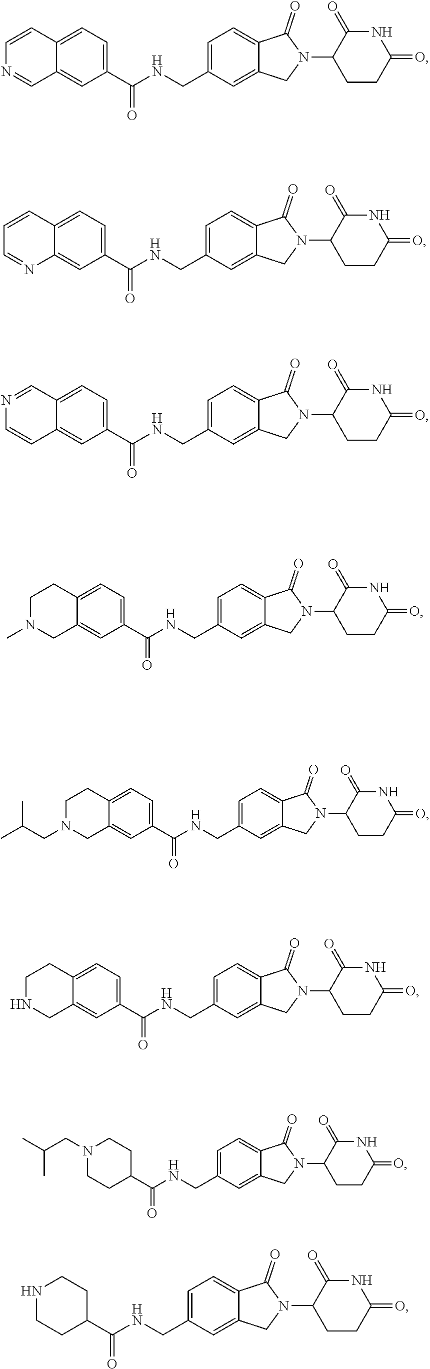

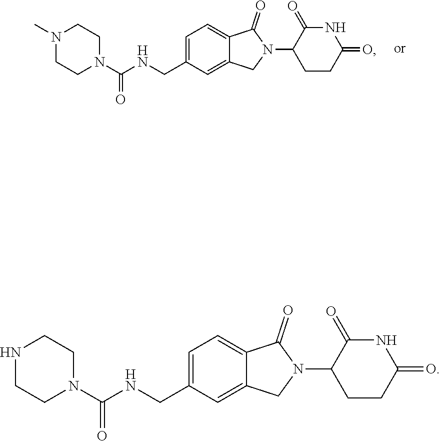 [SMILES: CN1CCN(C(=O)NCc2ccc3c(c2)CN(C2CCC(=O)NC2=O)C3=O)CC1 ; "or" ; O=C1CCC(N2Cc3cc(CNC(=O)N4CCNCC4)ccc3C2=O)C(=O)N1]

92. The method of claim 75, wherein Formula (a) is 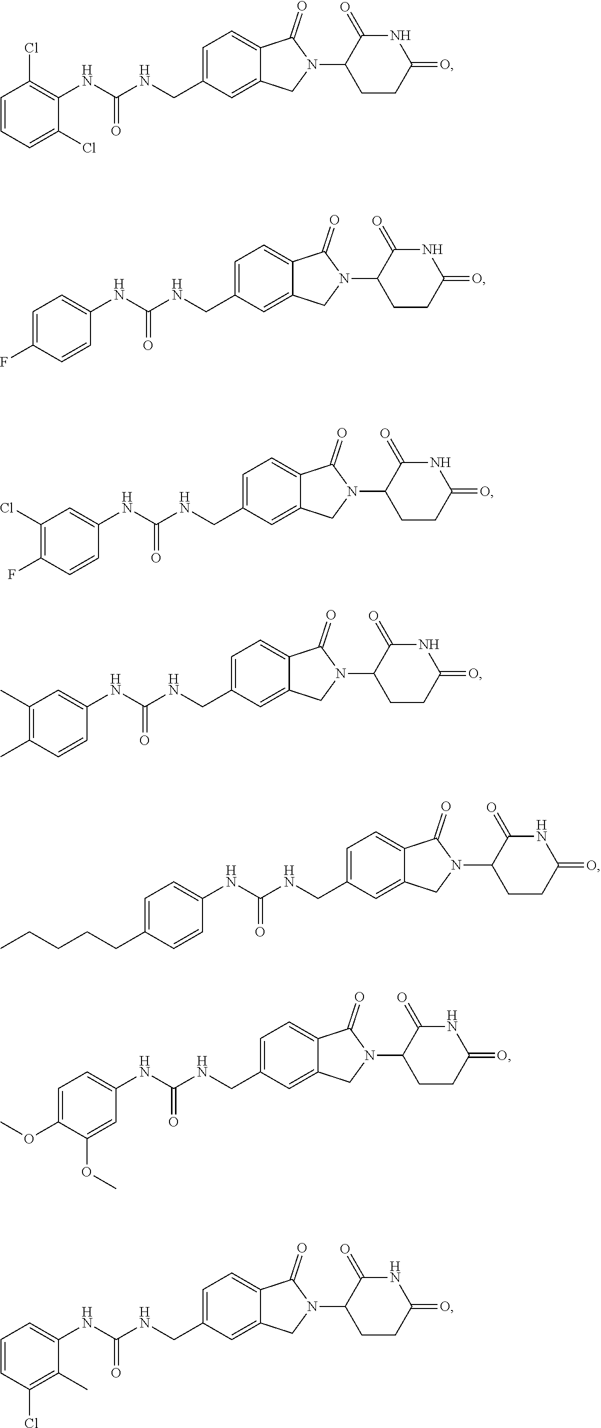 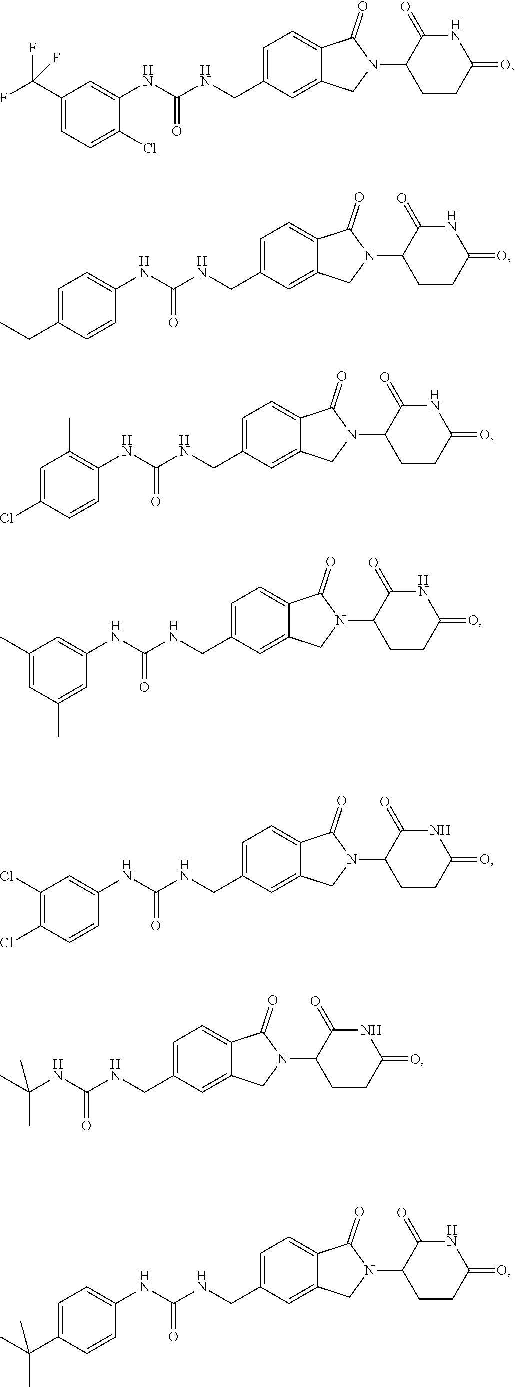 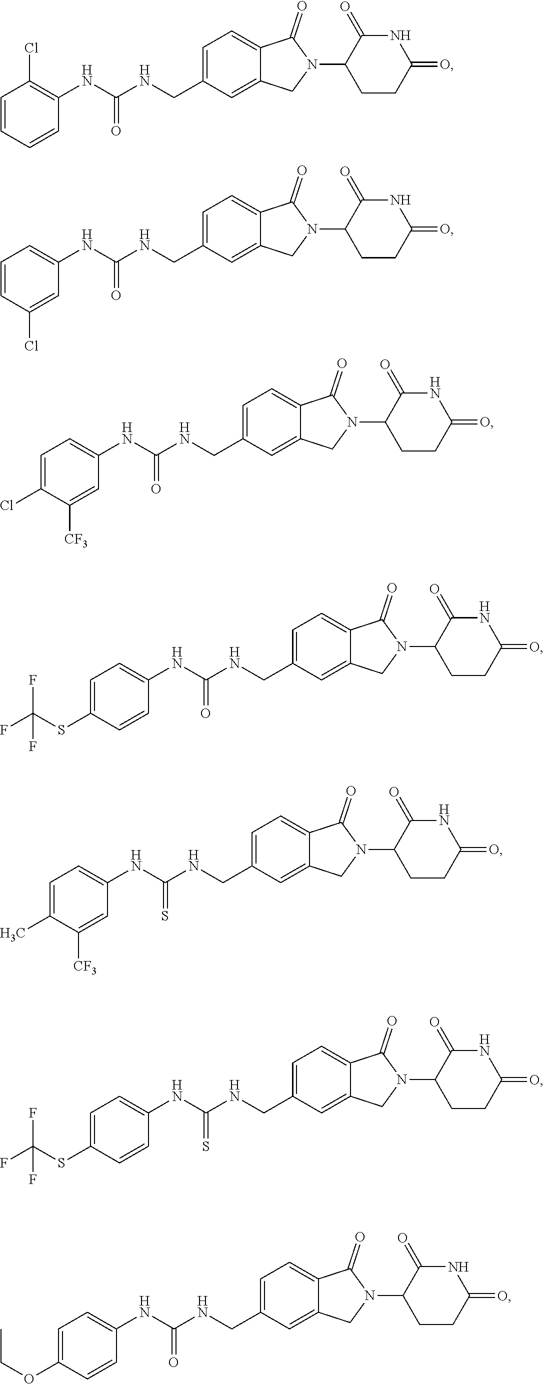 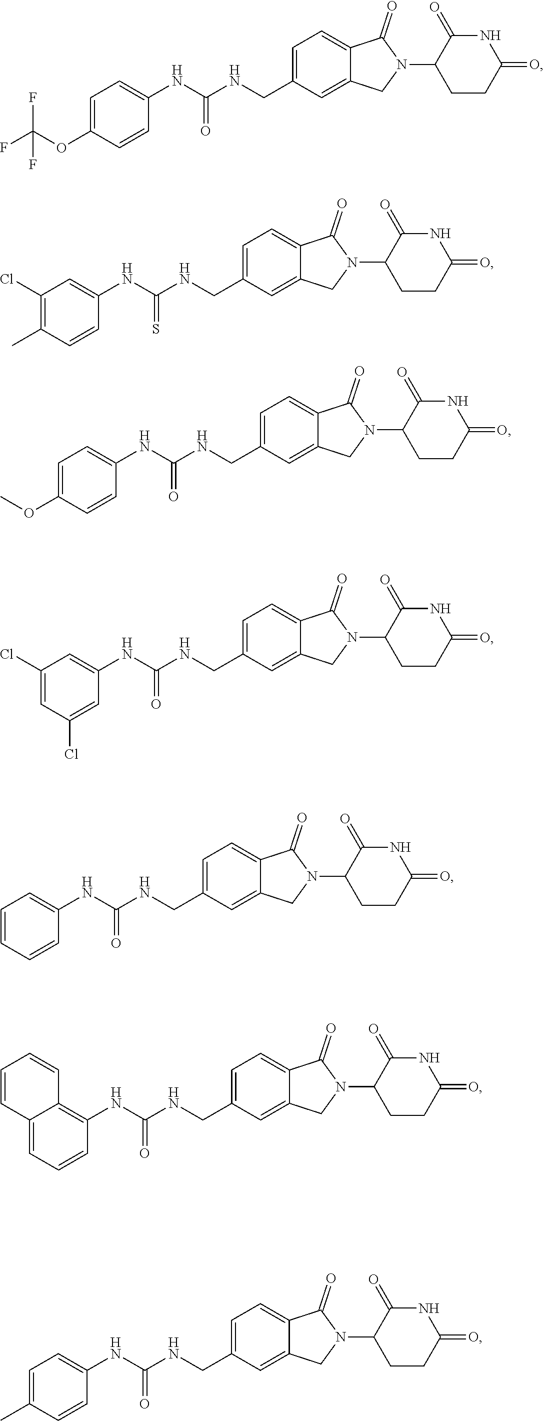 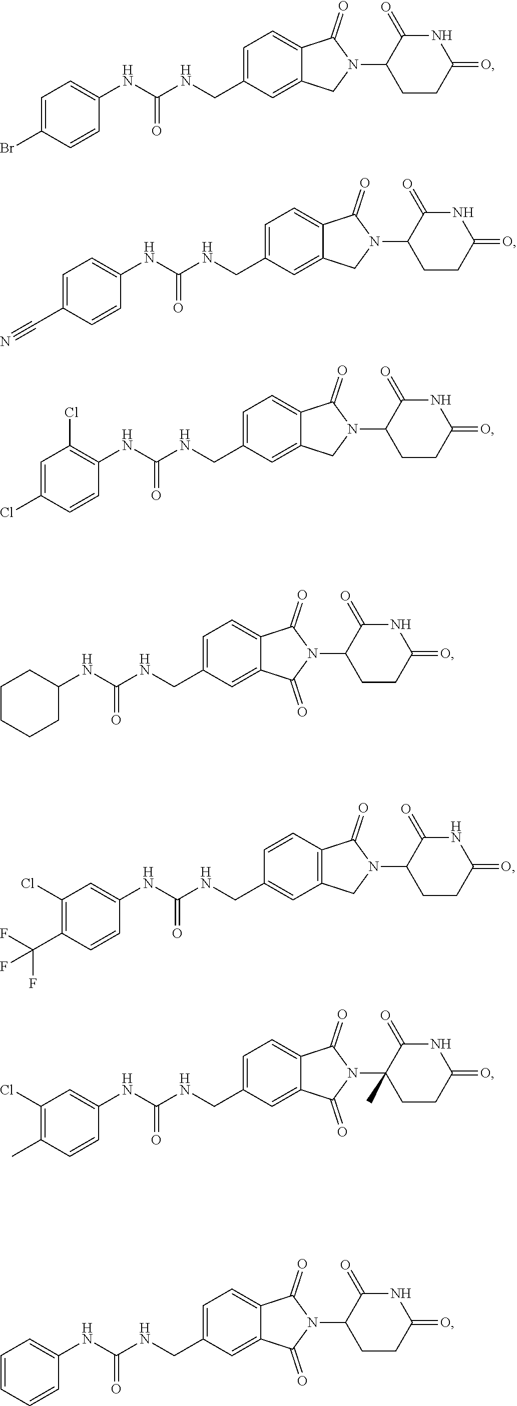 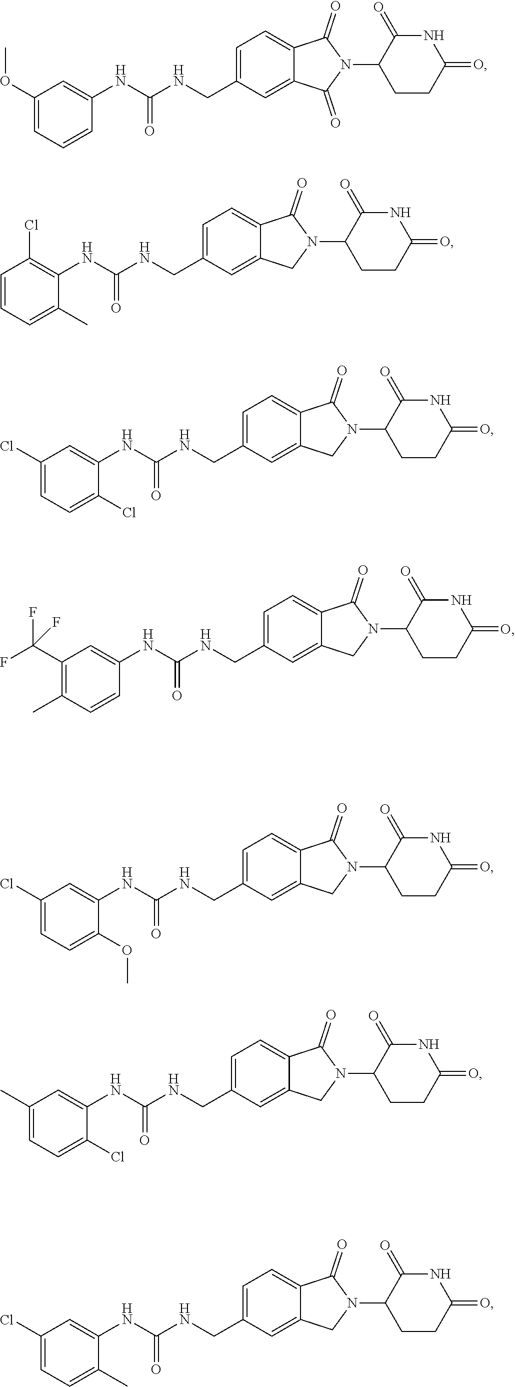 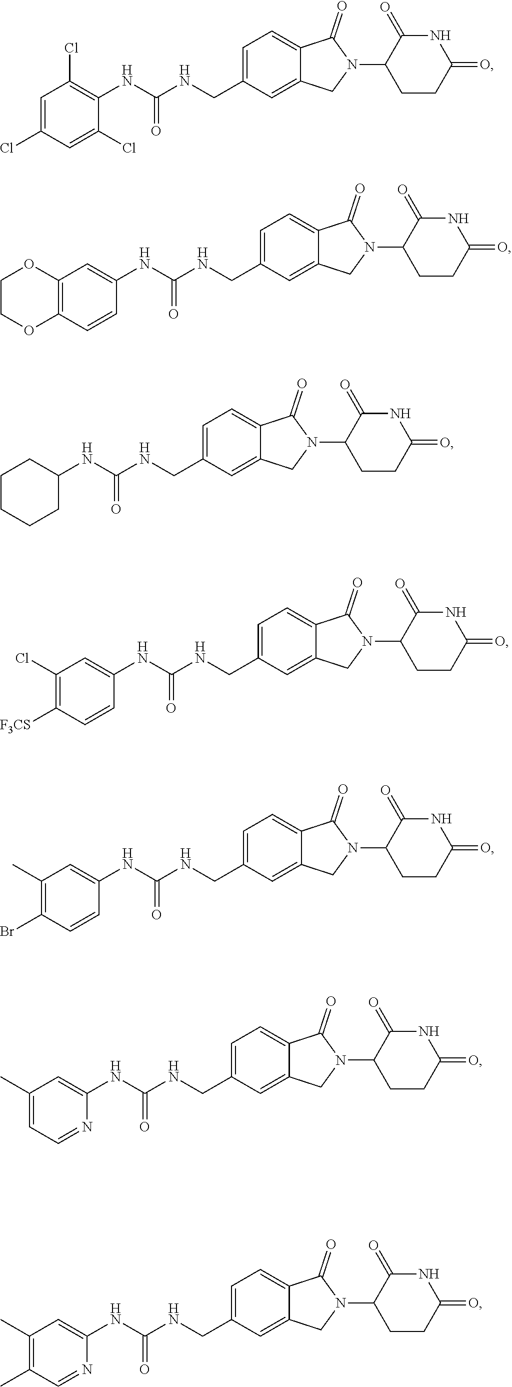 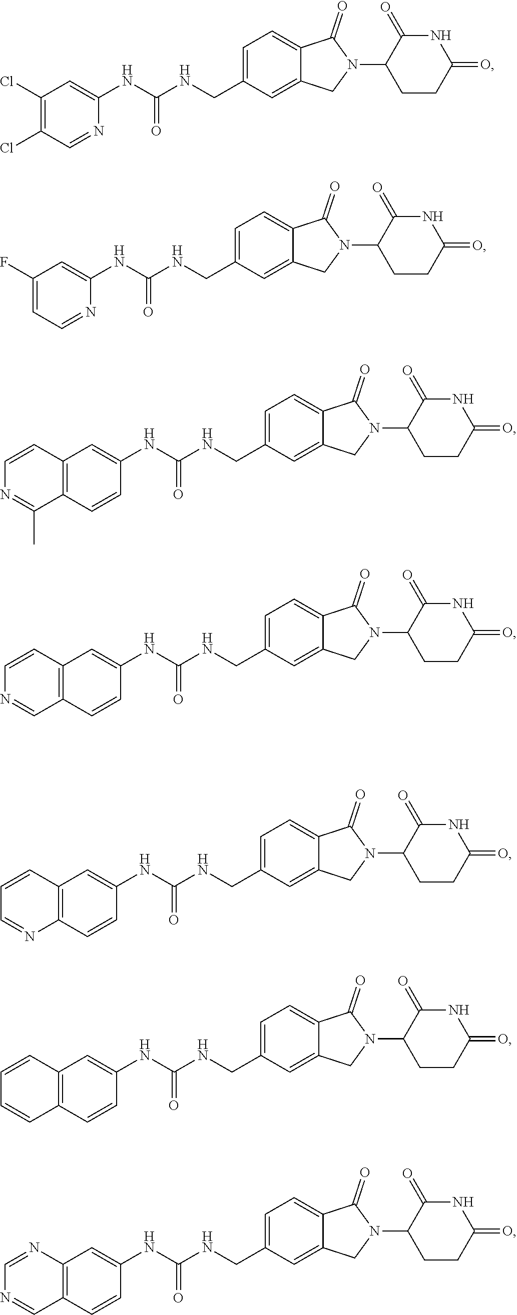 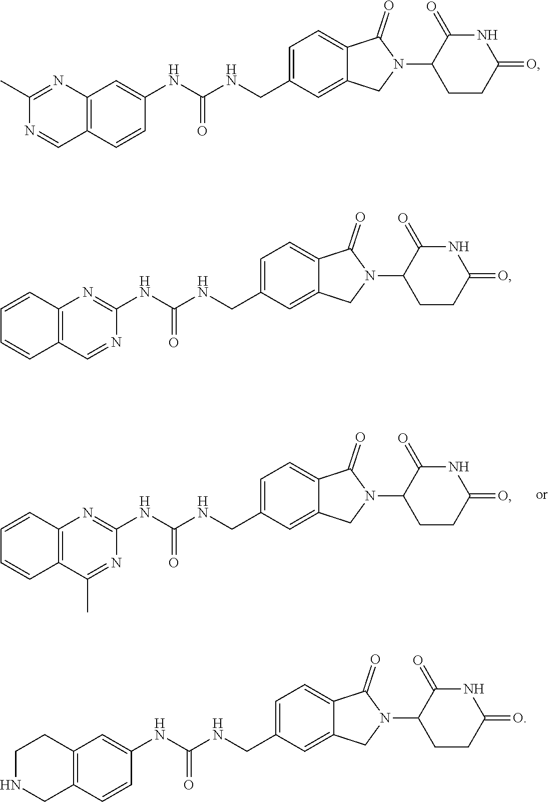 [SMILES: Cc1ncc2ccc(NC(=O)NCc3ccc4c(c3)CN(C3CCC(=O)NC3=O)C4=O)cc2n1 ; O=C1CCC(N2Cc3cc(CNC(=O)Nc4ncc5ccccc5n4)ccc3C2=O)C(=O)N1 ; Cc1nc(NC(=O)NCc2ccc3c(c2)CN(C2CCC(=O)NC2=O)C3=O)nc2ccccc12 ; "or" ; O=C1CCC(N2Cc3cc(CNC(=O)Nc4ccc5c(c4)CCNC5)ccc3C2=O)C(=O)N1]
